(19) United States
(12) Patent Application Publication
(10) Pub. No.: US 2024/0325308 A1
(43) Pub. Date: Oct. 3, 2024

(54) AMNIOTIC FLUID STABILIZED COMPOSITIONS AND METHODS FOR IN UTERO DELIVERY OF THERAPEUTIC AGENTS

(71) Applicants: The Trustees of the University of Pennsylvania, Philadelphia, PA (US); The Children's Hospital of Philadelphia, Philadelphia, PA (US)

(72) Inventors: Michael Mitchell, Philadelphia, PA (US); Kelsey L. Swingle, Philadephia, PA (US); Margaret M. Billingsley, Hockessin, DE (US); William Peranteau, Philadelphia, PA (US)

(73) Assignees: The Trustees of the University of Pennsylvania, Philadelphia, PA (US); The Children's Hospital of Philadelphia, Philadelphia, PA (US)

(21) Appl. No.: 18/293,419

(22) PCT Filed: Aug. 3, 2022
(86) PCT No.: PCT/US2022/074457

§ 371 Date: Jan. 30, 2024

Related U.S. Application Data
(60) Provisional application No. 63/229,168, filed on Aug. 4, 2021.

(51) Int. Cl. A61K 9/127; A61K 31/7105; G01N 33/50

(52) U.S. Cl. CPC A61K 9/1271; A61K 31/7105; G01N 33/5026; G01N 33/5076

(57) ABSTRACT

The present disclosure relates, in part, to lipid nanoparticles (LNPs), and compositions comprising the same, which have increased stability in amniotic fluid, and methods of use thereof for in utero delivery of therapeutic agents, to treat and/or prevent diseases and/or disorders in a fetal subject. The present disclosure further relates to an ex vivo assay for screening LNP stability in a test fluid.

### CROSS-REFERENCE TO RELATED APPLICATIONS

The present application claims priority under 35 U.S.C. § 119(e) to U.S. Provisional Application No. 63/229,168, filed Aug. 4, 2021, which application is hereby incorporated herein by reference in its entirety.

### STATEMENT REGARDING FEDERALLY SPONSORED RESEARCH OR DEVELOPMENT

This invention was made with government support under TR002776, DK123049, HL152427, and HL151352 awarded by the National Institutes of Health. The government has certain rights in the invention.

### BACKGROUND

Recent advances in prenatal care and genetic medicine have led to improvements in fetal diagnostics including fetal whole exome sequencing and non-invasive fetal genetic testing via detection of cell-free fetal DNA in maternal serum. This progress has enabled prenatal diagnosis of many genetic diseases such as β-thalassemia, cystic fibrosis, and glycogen storage disorders.

Although many genetic diseases can be optimally treated after birth, postnatal treatments have limited efficacy in diseases where the onset of irreversible pathology begins in utero. Instead, prenatal gene therapies, including protein replacement and gene editing therapeutics, allow for the treatment of congenital disorders prior to or in the early stages of pathology to reduce disease burden, morbidity, and mortality.

Additionally, there are a number of ontological properties of the developing fetus that result in practical and therapeutic advantages for prenatal gene therapy. First, the small size of the fetus allows for maximum dosing per fetal weight, therefore minimizing the challenges associated with the large-scale manufacture of gene therapies. Additionally, progenitor cells, which are an ideal target for genetic correction, are more abundant and accessible in utero, and physical barriers to delivery such as mucus membranes and glycocalyx are less developed in the fetus. With these notable advantages, some congenital diseases that are currently treated postnatally with protein or enzyme replacement therapeutics may be ideal candidates for prenatal gene therapy.

Two factors are critical to the delivery of gene therapies before or after birth—the delivery route and delivery vehicle. Multiple studies in small and large animal models have demonstrated the ability to target a number of different fetal organs by administering viral vectors via different injection routes.

First, intravenous injection of adenoviruses and adeno-associated viruses (AAVs) via the vitelline vein has demonstrated robust targeting to the fetal liver. Intramuscular injection of the fetal hindlimb resulted in efficient delivery of AAVs to the skeletal muscle. Intra-amniotic delivery of lentiviral vectors have been shown to target stem cells of a number of different organs in the developing fetus depending on the gestational age at which the vector was delivered. Similarly, late gestation intra-amniotic injection of viral vectors has been shown to target the fetal lungs and gastrointestinal tract by taking advantage of normal fetal breathing and swallowing movements.

Alternatively, in large animal models, fetal intra-tracheal injections can also directly target the lungs while avoiding technical difficulties that exist in small animal models and minimizing the dilutional effect of the large amniotic fluid volume on the therapeutic cargo.

Prenatal protein and enzyme replacement gene therapies can be administered via various delivery strategies. One option is the direct administration of whole proteins in utero, but these therapeutics are limited by the in vitro synthesis of proteins with the correct post-translational modifications. This challenge can be overcome by viral or non-viral delivery of nucleic acids which are instead translated endogenously in the host.

Viral vectors for the delivery of nucleic acids have shown promise in pre-natal applications, but pose the risk of genomic integration. Additional challenges of viral vectors such as immunogenicity and limitations regarding repeat dosing can be addressed with non-viral nucleic acid delivery. Non-viral nucleic acid delivery includes the administration of therapeutic messenger RNA (mRNA) which initiates transient protein expression in the cytosol and therefore avoids nuclear transport and the risk of genomic integration.

However, mRNA faces similar delivery challenges in utero as it does in adults, including rapid degradation by nucleases present in the body and inefficient transport across the cell membrane due to its large size and negative charge. These challenges have limited the broad clinical use of mRNA therapeutics and necessitate the development of mRNA delivery technologies for in vivo delivery.

Numerous delivery technologies have been investigated for the delivery of mRNA in vivo such as polymeric and lipid-based nanoparticle (NP) systems. One polymeric system, specifically poly(lactic-co-glycolic acid) (PLGA) NPs, has shown efficient delivery of gene editing nucleic acids to fetal hematopoietic stem cells for the prenatal treatment of β-thalassemia. However, other polymeric NPs for gene delivery that use highly cationic molecules such as polyethyleneimine (PEI) have been found to be highly toxic, therefore limiting their clinical application.

Instead, ionizable lipid nanoparticle (LNP) platforms can be used for the therapeutic delivery of mRNA, and they are more clinically advanced than polymeric systems following the recent Food and Drug Administration (FDA) approval of Alnylam's Onpattro siRNA LNP therapeutic and emergency use authorization of Moderna and Pfizer/BioNTech's mRNA vaccines against COVID-19. Ionizable LNPs benefit from high nucleic acid encapsulation efficiencies and small sizes (<100 nm) making them ideal vectors for in utero intracellular delivery. Additionally, LNPs contain an ionizable lipid component that remains neutral at physiological pH, but after cellular uptake, becomes charged in the acidic endosomal environment allowing for enhanced endosomal escape and potent intracellular mRNA delivery.

A final advantage of LNPs is their modular design; the excipients and their respective molar ratios, the ionizable lipid, and the lipid to nucleic acid ratio can all be individually optimized to improve biodistribution and intracellular delivery for a particular application. As LNP technology advances and the number of possible formulations continues to grow, there is a need for assays to evaluate and predict LNP performance for in vivo, including prenatal applications.

Recent work has demonstrated the substantial effect of the biological environment on NP stability, biodistribution, and delivery, yet these works have primarily focused on the effect of blood, serum, and simulated interstitial fluid biological environments. Fetal amniotic fluid, the biological environment for intra-amniotically administered in utero gene therapeutics, similar to serum, is a similar protein-rich environment as, which influences LNP stability and delivery.

There is a need in the art for improved compositions and methods for effective in utero delivery of agents to fetal cells, including mRNA-based therapeutics. The present disclosure satisfies this unmet need.

### BRIEF SUMMARY

In one aspect, the present disclosure provides a lipid nanoparticle comprising an ionizable lipid compound or salt thereof having the structure of any one of Formula (I)-Formula (XVI). In certain embodiments, the compound, or salt thereof, is present in a concentration range of about 10 mol % to about 50 mol %. In another aspect, the present disclosure provides a lipid nanoparticle comprising dioleoyl-phosphatidylethanolamine (DOPE). In certain embodiments, the DOPE is present in a concentration range of about 10 mol % to about 45 mol %. In one aspect, the present disclosure provides a lipid nanoparticle comprising a cholesterol lipid. In certain embodiments, the cholesterol lipid is present in a concentration range of about 5 mol % to about 50 mol %. In one aspect, the present disclosure provides a lipid nanoparticle comprising polyethylene glycol (PEG). In certain embodiments, the PEG is present in a concentration range of about 0.5 mol % to about 12.5 mol %. In certain embodiments, the chemical structure of the compounds of Formula (I)-Formula (XVI) are described herein.

In another aspect, the present disclosure provides a composition comprising at least one lipid nanoparticle of the present disclosure and at least one pharmaceutically acceptable carrier.

In another aspect, the present disclosure provides a method of delivering a nucleic acid molecule, therapeutic agent, or a combination thereof to a fetal subject in utero, the method comprising administering a therapeutically effectively amount of at least one LNP of the present disclosure or at least one composition of the present disclosure to a maternal subject comprising the fetal subject.

In another aspect, the present disclosure provides a method of delivering a nucleic acid molecule to a fetal cell, the method comprising in utero administration of a therapeutically effectively amount of at least one LNP of the present disclosure or at least one composition of the present disclosure comprising to a maternal subject comprising the fetal cell.

In another aspect, the present disclosure provides a method of preventing or treating a disease or disorder in a target fetal subject, the method comprising in utero administration of a therapeutically effectively amount of at least one LNP of the present disclosure or at least one composition of the present disclosure to a maternal subject comprising the fetal subject.

In another aspect, the present disclosure provides a method of identifying a LNP as having increased stability in a test fluid. In certain embodiments, the method comprises contacting at least one LNP with a first concentration of a test fluid. In certain embodiments, the method comprises determining the size and polydispersity index (PDI) of the LNP using dynamic light scattering (DLS). In certain embodiments, the method comprises contacting the at least one LNP to be tested with at least one additional concentration of the fluid. In certain embodiments, the method comprises determining the size and polydispersity index (PDI) of the LNP using dynamic light scattering (DLS) in the presence of the at least one additional concentration of the fluid. In certain embodiments, the method comprises comparing the size and polydispersity index (PDI) of the LNP at the at least two fluid concentrations. In certain embodiments, the method comprises identifying the test LNP as having stability in the test fluid based on the changes in size and polydispersity index (PDI) of the LNP.

In certain embodiments, the test fluid mimics a biological fluid.

In certain embodiments, the biological fluid is amniotic fluid or an amniotic fluid mimic.

In certain embodiments, the method is a screening method for detecting LNPs having increased stability.

In certain embodiments, the test LNP is identified as having stability in the test fluid based a lower level of change in at least one selected from the group consisting of size and PDI of the LNP as compared to a control LNP.

### BRIEF DESCRIPTION OF THE FIGURES

The drawings illustrate generally, by way of example, but not by way of limitation, various embodiments of the present application.

FIG. 1 shows an overview of the LNP library design, formulation, and ex utero screening in amniotic fluids to predict intra-amniotic delivery. A library of 16 LNP formulations was generated using orthogonal design of experiments (DOE) to explore four molar ratios of each of four excipients. Next, each LNP was synthesized by combining an ethanol phase of lipid excipients—including ionizable lipid, DOPE phospholipid, cholesterol, and lipid-PEG—with an aqueous phase containing luciferase mRNA. The two phases were mixed at controlled flow rates in a microfluidic device to form LNPs. Then, LNPs were screened ex utero in fetal fluids to identify stable and unstable LNPs to predict intra-amniotic delivery.

FIG. 2 depicts the chemical structures of the excipients used in LNP formulation. As described elsewhere herein, polyamine core 4 and C14 epoxide were reacted to synthesize the ionizable lipid used in LNP formulations. Other LNP excipients include DOPE (phospholipid), cholesterol, and the C14-PEG conjugate.

FIGS. 3A-3B depict exemplary experimental data demonstrating the ex utero LNP stability in mouse amniotic fluid. FIG. 3A depicts an exemplary stability assessment of LNPs A5 and A12 in mouse amniotic fluid with varying fluid percentages and incubation times. LNPs A5 and A12 were incubated in five percentages of mouse amniotic fluid—0%, 25%, 50%, 75%, and 100% (volume mouse amniotic fluid/total volume)—for 30 minutes. Intensity curves were recorded by DLS for both formulations across fluid percentages to demonstrate size and PDI changes. FIG. 3B depicts exemplary data demonstrating the size and PDI as measured by DLS for LNPs A5 and A12 at seven time points—0 min, 5 min, 15 min, 30 min, 60 min, 120 min, and 240 min—in 50% (v/v) mouse amniotic fluid. Size and PDI were measured by DLS for both formulations across timepoints. Data points are presented as means and standard deviations among n=3 to 4 technical replicates for each of three biological replicates.

FIGS. 4A-4D depict exemplary experimental data demonstrating LNP library stability in mouse serum and amniotic fluids. FIG. 4A provides a schematic depicting LNP library screen using DLS where percent change in LNP size or PDI in each amniotic fluid is calculated from the LNP size or PDI in PBS alone. These percent change measurements are compared to those in mouse serum as a positive control to identify hits. FIG. 4B provides exemplary heatmaps depicting log transforms of LNP percent change in size and percent change in PDI (from PBS) in each fluid. Red—darker colors represent larger percent changes in size from the LNP in PBS alone. Blue—darker colors represent larger percent changes in PDI from the LNP in PBS alone. FIGS. 4C-4D provide exemplary experimental 2-way ANOVA results indicating hits across amniotic fluids and formulations for percent change in size (FIG. 4C) and percent change in PDI (FIG. 4D) measurements. A hit is defined as an LNP in a given amniotic fluid with a significantly smaller (p<0.05) percent change in size or PDI measurement than the LNP in mouse serum as determined from 2-way ANOVA.

FIGS. 5A-5E depict exemplary experimental data demonstrating representative intensity (%) vs. size (nm) curves for most and least stable LNPs in each of the five fluids evaluated: mouse serum (FIG. 5A), mouse amniotic (FIG. 5B), sheep amniotic (FIG. 5C), pig amniotic (FIG. 5D), and human amniotic (FIG. 5E). Most stable particles in PBS alone (dashed) and with each fluid (solid) are shown in green. Least stable particles in PBS alone (dashed) and with each fluid (solid) are shown in purple. The background intensity curve of each fluid is shown in black.

FIGS. 6A-6B depicts exemplary experimental data demonstrating LNP instability parameter correlations for amniotic fluids across species. FIG. 6A depicts exemplary data demonstrating instability parameter measurements of the LNP library in mouse, sheep, and pig amniotic fluids (y axis) correlated with human amniotic fluid (x axis). FIG. 6B depicts exemplary data demonstrating instability parameter measurements of the LNP library in sheep and pig amniotic fluids (y axis) correlated with mouse amniotic fluid (x axis). The coefficient of determination R2 of the least squares linear regressions indicate the goodness of fit for the instability parameter correlations.

FIGS. 7A-7B depict exemplary experimental data demonstrating the effect of different ionizable lipids for A5 formulation on ex vivo size and PDI stability measurements. Two A5 LNPs were formulated with either the B-4 or C12-200 ionizable lipids. FIG. 7A depicts data demonstrating that there was no significant difference (p<0.05) in ex vivo size measurements for A5 LNP formulated with B-4 ionizable lipid versus C12-200 ionizable lipid in any of the amniotic fluids tested. FIG. 7B depicts data demonstrating that A5 LNP formulated with C12-200 ionizable lipid had significantly higher PDI measurements in PBS (*p<0.05), mouse serum (*p<0.05), and pig amniotic (***p<0.001) fluids than the same LNP with B-4 ionizable lipid. These results in fluids are likely explained by the significantly higher PDI of the C12-200 LNP in PBS alone compared to the same formulation with B-4 ionizable lipid.

FIGS. 8A-8E depict exemplary experimental data demonstrating LNP morphology and protein interactions in mouse amniotic fluid. FIG. 8A depicts TEM images of the most stable (A12) and least stable (A1) LNPs from ex utero mouse amniotic fluid screen. FIG. 8B depicts the zeta potential of A12 and A1 LNPs with increasing percentages (v/v) of mouse amniotic fluid. FIGS. 8C-8E depict an exemplary BCA assay identifying protein content bound to LNPs following LNP incubation in mouse amniotic fluid and chromatographic separation of LNPs from unbound mouse amniotic fluid. Data is presented as means with standard deviations of n=3 to 4 measurements.

FIGS. 9A-9D depict exemplary experimental data demonstrating in vitro LNP-mediated luciferase mRNA delivery. FIG. 9A depicts LNP-mediated luciferase mRNA delivery in HeLa cells. Cells were treated with the 16 LNP library in PBS alone and pre-incubated in mouse amniotic fluid. Luciferase expression for each treatment condition was normalized to untreated cells and compared to lipofectamine MessengerMAX delivery using 2-way ANOVA for significance. Seven LNPs with mouse amniotic fluid had significant (*p<0.05) delivery compared to lipofectamine. Only LNP A7 had significantly different delivery with mouse amniotic fluid compared to the same formulation in PBS alone. FIG. 9B depicts data demonstrating the cell viability following treatment with the LNP library in PBS alone or pre-incubated in mouse amniotic fluid. LNPs A1, A7, and A8 had significantly (*p<0.05) lower cell viability compared to lipofectamine. LNPs A7, A13, and A14 had significantly better cell viability after pre-incubation in mouse amniotic fluid compared to the same formulation in PBS alone. FIG. 9C depicts data demonstrating the correlation between encapsulation efficiency and luciferase delivery. FIG. 9D depicts data demonstrating the inverse correlation between luciferase expression and percent change in size (left) and percent change in PDI (right) in mouse amniotic fluid. Particles with encapsulation efficiencies≥75% excluded from correlation.

FIGS. 10A-10D depict exemplary experimental data demonstrating LNP structure function relationships with ex utero stability and in vitro delivery. Each data point represents an average of the stability and luciferase expression measurements of the four LNPs with the given excipient molar ratio. FIG. 10A depicts data demonstrating the percent change in size and PDI decrease as ionizable lipid B-4 increases. FIGS. 10B-10C depict data demonstrating the percent change in PDI decreases and luciferase expression increases as the molar ratio of DOPE and cholesterol increases. FIG. 10D depicts data demonstrating the percent change in PDI increases and luciferase expression decreases as the molar ratio of PEG increases.

FIGS. 11A-11D depict exemplary experimental data demonstrating LNP-mediated intra-amniotic luciferase mRNA delivery. Two LNPs—A12 and A4—were selected to evaluate in utero luciferase mRNA delivery. FIG. 11A depicts a schematic of intra-amniotic injection. FIG. 11B depicts data demonstrating an exemplary IVIS image of dam and exposed uterine horn with pups in the four left sacs receiving PBS control and pups in the five right sacs receiving A4 LNP injections (left) and strong luciferase expression in the uterine horn where pups received A12 LNP injections, other than one sac (denoted with white arrow) that instead received PBS as a control injection (right). FIG. 11C depicts IVIS images (left) and quantification (right) of fetal bioluminescence after surgical removal from the dams. IVIS images indicate variability in luciferase expression for A12 LNP condition, with the luciferase expression from one fetus identified as an outlier (denoted with an X) and removed from analysis. A12 LNP had significantly higher fetal luciferase expression, as quantified by normalized total flux, compared to both A4 LNP and PBS control injections. FIG. 11D depicts IVIS images (left) of the highest luciferase expression in each organ for all conditions. Quantification (right) of fetal organ bioluminescence following dissection. There was no significant difference in the normalized total flux for A12 LNP compared to A4 LNP or PBS control across four organs shown.

### DETAILED DESCRIPTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Description

In the present work, an ex utero screening platform was developed to assess LNP stability in various fetal fluid biological environments and the ability of this assay to predict LNP-mediated in vitro and in utero luciferase mRNA delivery was demonstrated. By using DLS and measured changes in LNP size and PDI following incubation in fluid, this stability assay requires minimal LNP and fluid resources. As a result, a larger number of formulations can be evaluated using this ex vivo approach than in other labor intensive and expensive in vivo screening experiments. For example, here, the ex vivo screening of a 16 LNP library identified excipient formulations in mouse, sheep, pig, and human amniotic fluids that were highly stable. Future ex vivo screening could include design of a second generation library for each fluid of interest to further optimize LNP stability.

Library screening and establishing structure function relationships between LNP formulation and delivery are increasingly valuable as the number of possible formulations continues to grow with research on new modular LNP components. For example, recent work has shown that varying the molar ratio of cationic lipids such as DOTAP in LNP formulations can shift organ biodistribution in an effort to improve delivery to a target of interest (Cheng et al., 2020, Nat. Nanotechnol. 15:313-320). Previous work has also demonstrated that different phospholipids such as DOPE and DSPC, or different ratios of lipid to nucleic acid cargo can improve encapsulation and delivery of one or multiple nucleic acids (Ball et al., 2018, Nano Lett. 9). While the reproducible nature of this assay is demonstrated with different ionizable lipids, without being bound to theory, it was hypothesized that modular changes in LNP formulation could impact ex vivo LNP stability.

Like in this present study, mice are often used to evaluate in utero therapeutic delivery due to their short gestational period (approximately 20 days) and ability to simultaneously carry multiple fetuses per dam (Figueroa-Espada et al., 2020, Adv. Drug Deliv. Rev. 160:244-261). However, the small size of the mouse fetus presents technical challenges with respect to evaluating delivery approaches that would be possible in humans. In these circumstances, preclinical, large animal models may provide valuable information. These larger animal models, including time-dated pregnant pigs and sheep, are labor and cost prohibitive and are only used for well-characterized and clinically translatable technologies, therefore limiting in vivo LNP optimization in these species. However, the longer gestational period of sheep (approximately 145 days) in contrast to that of the mouse (approximately 20 days) more closely mimics the development period of the human fetus. This may be one explanation for the stronger correlation of LNP stability measurements in sheep and human amniotic fluids in contrast to mouse amniotic fluid. To combat the challenges associated with differences between small and large animal models, the ex utero stability screening platform established in this study enables identification of novel LNP formulations specifically for larger species using unique biological environments such as fetal amniotic fluid.

Beyond using DLS to characterize size and PDI changes upon incubation in fluid, morphological and protein effects were characterized to further understand ex utero LNP stability. Upon incubation in mouse amniotic fluid, the least stable LNP from the ex utero mouse amniotic fluid stability screen exhibited substantial morphological changes, including aggregation and increased size. Instead, the most stable LNP in mouse amniotic fluid presented little morphological changes, with only a small increase in size. These morphological differences visualized in TEM images confirmed what was found in the ex utero stability screen where more stable LNPs had little size or PDI changes upon incubation in fluids, but less stable particles exhibited substantial size and PDI changes from the LNPs in PBS alone.

In certain non-limiting embodiments, these differences in stability may be due to differences in the amount of bound protein on the LNP surface, as the fluids evaluated in this study are protein-rich biological environments. However, contrary to what was expected, the more stable LNP in mouse amniotic fluid had significantly higher protein content bound to the surface than the least stable LNP from the ex utero stability screen. This finding demonstrates some LNP structure function relationship that makes certain formulations better suited to resist conformational changes in protein rich biological environments. Without being bound to theory, these findings can be due to different types of protein coronas that form on the surface of nanoparticles in biological fluids. In general, protein coronas are considered to be the sum of all the proteins that adsorb on the surface of nanoparticles such as LNPs when they come in contact with a protein-rich biological environment such as serum or amniotic fluid (Francia et al., 2020, Bioconjug. Chem. 31:2046-2059). Types of protein coronas include “hard” coronas which represent proteins that bind directly to the LNP surface with high affinity, while “soft” coronas are considered a looser, more dynamic protein layer that interacts more freely with the biological environment (Francia et al., 2020, Bioconjug. Chem. 31:2046-2059). Less stable LNPs are potentially forming primarily hard protein coronas that more substantially impact LNP conformation and resulting size and PDI measurements using DLS. Instead, more stable LNPs may be forming primarily soft protein coronas with reversibly bound proteins, consequently resulting in less substantial conformational changes as measured by size and PDI measurements (Chen et al., 2017, Nanomed. 12:2113-2135).

Besides LNP stability, the biological environment can also impact in vitro delivery. Previously, it has been proposed that protein coronas can have paradoxical effects on in vitro cellular uptake and LNP delivery (Chen et al., 2017, Nanomed. 12:2113-2135). For example, while proteins on the LNP surface can trigger and enhance cellular uptake, specifically via protein-receptor interactions, proteins bound to the LNP surface can also decrease LNP adhesion to the cell membrane due to surface free-energy limitations, therefore decreasing LNP uptake (Chen et al., 2017, Nanomed. 12:2113-2135). Here no significant difference was found in LNP mediated luciferase mRNA delivery for 15 of the 16 LNPs in the presence of mouse amniotic fluid compared to each LNP in PBS alone. Interestingly though, improved cell viability was identified for three LNPs in the presence of mouse amniotic fluid compared to the LNP in PBS alone.

Structure function relationships were evaluated between LNP excipient molar ratios and their ex utero stability and in vitro luciferase mRNA delivery in mouse amniotic fluid. These relationships indicated that the percent change in PDI stability measurements more closely tracked as expected with mRNA delivery than percent change in size stability measurements. In other words, LNP percent change in PDI measurements and LNP-mediated luciferase mRNA delivery were inversely related as expected. As changes in PDI are representative of changes in size distribution, it is likely that large PDIs indicate the presence of both large LNP aggregates and small broken down LNPs due to high protein content on the LNP surface. Perhaps, these LNP distribution changes are more indicative of functional delivery in a protein-rich environment than increases in LNP size. Additionally, the inverse relationship between percent change in PDI measurements and mRNA delivery was especially strong when observing variations in molar ratio of PEG. This is an interesting observation as PEG is often included in LNP formulations to reduce immune system recognition and rapid clearance that is often initiated by protein adhesion to the LNP surface (Oberli et al., 2017, Nano Lett. 17:1326-1335). However, it was found that increased PEG appears to be detrimental to LNP stability and functional delivery in mouse amniotic fluid. An optimized molar ratio of PEG is potentially necessary to limit LNP protein adhesion to the surface and consequent immune system activation and LNP clearance. Like PEG, additional advancements in LNP technology include the surface conjugation of targeting moieties such as peptides or antibody fragments (Kedmi et al., 2018, Nat. Nanotechnol. 13:214-219) which are similarly expected to extend from the surface of LNPs. Therefore, targeted LNP technologies might also benefit from ex vivo stability evaluations to further understand LNP structure function relationships.

Finally, intra-amniotic delivery of a highly stable and less stable LNP from the ex utero mouse amniotic stability screen demonstrated significantly increased in utero luciferase mRNA delivery for the more stable LNP compared to the less stable LNP. As hypothesized based on the timing of injection during mouse development when fetal breathing and swallowing movements are active, the intra-amniotically injected LNPs demonstrated some signal in the intestine and lung. Additional bioluminescent signal in the fetal images could represent some LNP-mediated luciferase mRNA delivery to fetal membranes within the amniotic sac or the fetal skin. Additionally, substantial luciferase expression variability was noted amongst the fetuses receiving the most stable LNP treatment. Ultimately, these in vivo results demonstrate the ability of ex utero LNP stability to predict LNP mediated in utero luciferase mRNA delivery.

In conclusion, here an ex utero LNP stability screening platform has been developed to identify novel LNP formulations for enhanced stability in a series of amniotic fluids. This work can be continued to further optimize formulations for the treatment of congenital diseases in utero, or explored with other protein-rich biological fluids for different organ and disease target applications. Overall, this study demonstrates the ability of ex utero stability measurements to predict in utero luciferase mRNA delivery. The ex utero stability measurements can serve as a low resource optimization tool to improve the delivery efficacy of gene therapy LNP technologies.

### Definitions

The term “about” as used herein can allow for a degree of variability in a value or range, for example, within 10%, within 5%, or within 1% of a stated value or of a stated limit of a range, and includes the exact stated value or range.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “adjuvant” as used herein is defined as any molecule to enhance an antigen-specific adaptive immune response.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═C═CCH₂, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The term “antibody,” as used herein, refers to an immunoglobulin molecule, which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present disclosure may exist in a variety of forms including, for example, polyclonal antibodies, monoclonal antibodies, Fv, Fab and F(ab)₂, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. κ and λ light chains refer to the two major antibody light chain isotypes.

The term “antigen” or “Ag” as used herein is defined as a molecule that provokes an adaptive immune response. This immune response may involve either antibody production, or the activation of specific immunogenically-competent cells, or both. The skilled artisan will understand that any macromolecule, including virtually all proteins or peptides, can serve as an antigen. Furthermore, antigens can be derived from recombinant or genomic DNA or RNA. A skilled artisan will understand that any DNA or RNA, which comprises a nucleotide sequences or a partial nucleotide sequence encoding a protein that elicits an adaptive immune response therefore encodes an “antigen” as that term is used herein. Furthermore, one skilled in the art will understand that an antigen need not be encoded solely by a full length nucleotide sequence of a gene. It is readily apparent that the present disclosure includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “atm” as used herein refers to a pressure in atmospheres under standard conditions. Thus, 1 atm is a pressure of 101 kPa, 2 atm is a pressure of 202 kPa, and so on.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbomyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbomyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The term “disease” refers to a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The terms “epoxy-functional” or “epoxy-substituted” as used herein refers to a functional group in which an oxygen atom, the epoxy substituent, is directly attached to two adjacent carbon atoms of a carbon chain or ring system. Examples of epoxy-substituted functional groups include, but are not limited to, 2,3-epoxypropyl, 3,4-epoxybutyl, 4,5-epoxypentyl, 2,3-epoxypropoxy, epoxypropoxypropyl, 2-glycidoxyethyl, 3-glycidoxypropyl, 4-glycidoxybutyl, 2-(glycidoxycarbonyl)propyl, 3-(3,4-epoxycylohexyl)propyl, 2-(3,4-epoxy cyclohexyl)ethyl, 2-(2,3-epoxy cylopentyl)ethyl, 2-(4-methyl-3,4-epoxycyclohexyl)propyl, 2-(3,4-epoxy-3-methylcylohexyl)-2-methylethyl, and 5,6-epoxyhexyl.

The term “effective amount” as used herein, means an amount which provides a therapeutic or prophylactic benefit.

The term “encoding” as used herein refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “expression vector” as used herein refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) RNA, and viruses (e.g., lentiviruses, retroviruses, adenoviruses, and adeno-associated viruses) that incorporate the recombinant polynucleotide.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heteroarylalkyl” as used herein refers to alkyl groups as defined herein in which a hydrogen or carbon bond of an alkyl group is replaced with a bond to a heteroaryl group as defined herein.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

The term “homologous” as used herein, refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared×100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group.

The term “immunogen” as used herein refers to any substance introduced into the body in order to generate an immune response. That substance can a physical molecule, such as a protein, or can be encoded by a vector, such as DNA, mRNA, or a virus.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “isolated” as used herein means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

By the term “modulating” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

The term “monovalent” as used herein refers to a substituent connecting via a single bond to a substituted molecule. When a substituent is monovalent, such as, for example, F or Cl, it is bonded to the atom it is substituting by a single bond.

Unless otherwise specified, the term “nucleotide sequence encoding an amino acid sequence” includes all nucleotide sequences that are degenerate versions of each other and that encode the same amino acid sequence. Nucleotide sequences that encode proteins and RNA may include introns. In addition, the nucleotide sequence may contain modified nucleosides that are capable of being translation by translational machinery in a cell. For example, an mRNA where all of the uridines have been replaced with pseudouridine, 1-methyl psuedouridien, or another modified nucleoside.

The term “operably linked” as used herein refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA or RNA sequences are contiguous and, where necessary to join two protein coding regions, in the same reading frame.

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The terms “patient,” subject,” “individual,” and the like, are used interchangeably herein, and refer to any animal, or cells thereof whether in vitro or in situ, amenable to the methods described herein. In certain non-limiting embodiments, the patient, subject or individual is a human.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

In certain instances, the polynucleotide or nucleic acid of the disclosure is a “nucleoside-modified nucleic acid,” which refers to a nucleic acid comprising at least one modified nucleoside. A “modified nucleoside” refers to a nucleoside with a modification. For example, over one hundred different nucleoside modifications have been identified in RNA (Rozenski, et al., 1999, The RNA Modification Database: 1999 update. Nucl Acids Res 27: 196-197).

In certain embodiments, “pseudouridine” refers, In other embodiments, to m¹acp³Ψ (1-methyl-3-(3-amino-3-carboxypropyl) pseudouridine. In other embodiments, the term refers to m¹Ψ (1-methylpseudouridine). In other embodiments, the term refers to Tm (2′-O-methylpseudouridine. In other embodiments, the term refers to m⁵D (5-methyldihydrouridine). In other embodiments, the term refers to m³Ψ (3-methylpseudouridine). In other embodiments, the term refers to a pseudouridine moiety that is not further modified. In other embodiments, the term refers to a monophosphate, diphosphate, or triphosphate of any of the above pseudouridines. In other embodiments, the term refers to any other pseudouridine known in the art. Each possibility represents a separate embodiment of the present disclosure.

The terms “peptide,” “polypeptide,” and “protein” are used interchangeably herein, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence. For example, the promoter that is recognized by bacteriophage RNA polymerase and is used to generate the mRNA by in vitro transcription.

The term “room temperature” as used herein refers to a temperature of about 15° C. to 28° C.

The term “solvent” as used herein refers to a liquid that can dissolve a solid, liquid, or gas. Non-limiting examples of solvents are silicones, organic compounds, water, alcohols, ionic liquids, and supercritical fluids.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more other species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀) hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

The term “synthetic antibody” as used herein, is meant an antibody, which is generated using recombinant DNA technology, such as, for example, an antibody expressed by a bacteriophage. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art. The term should also be construed to mean an antibody, which has been generated by the synthesis of an RNA molecule encoding the antibody. The RNA molecule expresses an antibody protein, or an amino acid sequence specifying the antibody, wherein the RNA has been obtained by transcribing DNA (synthetic or cloned) or other technology, which is available and well known in the art.

The term “therapeutic” as used herein means a treatment and/or prophylaxis. A therapeutic effect is obtained by suppression, diminution, remission, or eradication of at least one sign or symptom of a disease or disorder state.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated.

To “treat” a disease as the term is used herein, means to reduce the frequency or severity of at least one sign or symptom of a disease or disorder experienced by a subject.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

The term “vector” as used herein refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

In the context of the present disclosure, the following abbreviations for the commonly occurring nucleosides (nucleobase bound to ribose or deoxyribose sugar via N-glycosidic linkage) are used. “A” refers to adenosine, “C” refers to cytidine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

### Lipid Nanoparticles (LNPs)

The present disclosure relates to compositions comprising ionizable LNP molecules formulated for in utero stability and methods of use thereof for in utero delivery of an encapsulated agent. Exemplary agents that can be encapsulated in the compositions of the disclosure include, but are not limited to, diagnostic agents, detectable agents, and therapeutic agents. In some embodiments, the encapsulated agent comprises an agent for genetic engineering of a target fetal subject. In certain embodiments, the present disclosure provides a composition comprising an ionizable LNP molecule formulated for in utero stability, and encapsulating a nucleic acid molecule encoding an agent for genetic engineering of a target fetal subject.

In one aspect, the present disclosure relates to a lipid nanoparticle comprising an ionizable lipid compound or salt thereof having the structure of any one of Formula (I)-Formula (XVI), wherein in certain embodiments the compound, or salt thereof, is present in a concentration range of about 10 mol % to about 50 mol %. In one aspect, the present disclosure relates to a lipid nanoparticle comprising dioleoyl-phosphatidylethanolamine (DOPE), wherein in certain embodiments the DOPE is present in a concentration range of about 10 mol % to about 45 mol %. In one aspect, the present disclosure relates to a lipid nanoparticle comprising a cholesterol lipid, which in certain embodiments is in a concentration range of about 5 mol % to about 50 mol %. In one aspect, the present disclosure relates to a lipid nanoparticle comprising polyethylene glycol (PEG), which in certain embodiments is in a concentration range of about 0.5 mol % to about 12.5 mol %.

In certain embodiments, the ionizable lipid compound or salt thereof is a compound of formula (I):

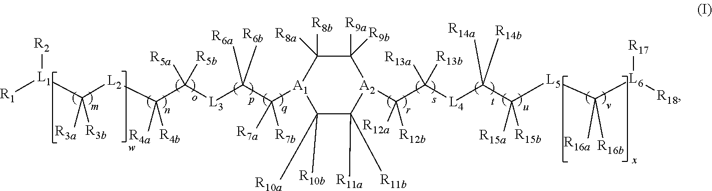

wherein:
A₁ and A₂ is independently selected from the group consisting of C(H) and N;
L₁ and L₆ are each independently selected from the group consisting of C(R₁₉) and N; each occurrence of L₂, L₃, L₄, and L₅, is independently selected from the group consisting of —C(H)₂—, —C(H)(R₁₉)—, —O—, —N(H)—, and —N(R₁₉)—;
each occurrence of R₁, R₂, R₃ₐ, R_{3b}, R₄ₐ, R_{4b}, R₅ₐ, R_{5b}, R₆ₐ, R_{6b}, R₇ₐ, R_{7b}, R₈ₐ, R_{8b}, R₉ₐ, R_{9b}, R₁₀ₐ, R_{10b}, R₁₁ₐ, R_{11b}, R₁₂ₐ, R_{12b}, R₁₃ₐ, R_{13b}, R₁₄ₐ, R_{14b}, R₁₅ₐ, R_{15b}, R₁₆, R₁₇, R₁₈, and R₁₉ is independently selected from the group consisting of H, OH, ═O, CN, NO₂, halogen, optionally substituted C₁-C₂₄ alkyl, optionally substituted C₃-C₁₂ cycloalkyl, —Y(R₂₀)_{z′}(R₂₁)_{z″}— (optionally substituted C₃-C₁₂ cycloalkyl), optionally substituted C₂-C₁₂ heterocycloalkyl, —Y(R₂₀)_{z′}(R₂₁)_{z″}-(optionally substituted C₂-C₁₂ heterocycloalkyl), optionally substituted C₂-C₂₄ alkenyl, optionally substituted C₅-C₁₂ cycloalkenyl, —Y(R₂₀)_{z′}(R₂₁)_{z″}-(optionally substituted C₂-C₁₂ cycloalkenyl), optionally substituted C₂-C₂₄ alkynyl, optionally substituted C₈-C₁₂ cycloalkynyl, —Y(R₂₀)_{z′}(R₂₁)_{z″}-(optionally substituted C₅-C₁₂ cycloalkynyl), optionally substituted C₆-C₁₂ aryl, —Y(R₂₀)_{z′}(R₂₁)_{z″}-(optionally substituted C₆-C₁₂ aryl), optionally substituted C₂-C₁₂ heteroaryl, —Y(R₂₀)_{z′}(R₂₁)_{z″}-(optionally substituted C₂-C₁₂ heteroaryl), —C(═O)OH, —C(═O)O(optionally substituted C₁-C₂₄ alkyl), —C(═O)O(optionally substituted C₂-C₂₄ alkenyl), —C(═O)O(optionally substituted C₆-C₁₂ aryl), —C(═O)O(optionally substituted C₂-C₁₂ heteroaryl), amido, amino, —Y(R₂₀)_{z′}(R₂₁)_{z″}—C(═O)O(optionally substituted C₁-C₂₄ alkyl), —Y(R₂₀)_{z′}(R₂₁)_{z″}—C(═O)O(optionally substituted C₂-C₂₄ alkenyl), —Y(R₂₀)_{z′}(R₂₁)_{z″} C(═O)O(optionally substituted C₆-C₁₂ aryl), and —Y(R₂₀)_{z′}(R₂₁)_{z″}—C(═O)O(optionally substituted C₂-C₁₂ heteroaryl);
wherein Y is selected from the group consisting of C, N, O, S, and P;
wherein each R₂₀ and R₂₁ is independently selected from the group consisting of H, OH, ═O, NO₂, CN, halogen, optionally substituted C₁-C₂₄ alkyl, optionally substituted C₃-C₁₂ cycloalkyl, optionally substituted C₂-C₁₂ heterocycloalkyl, optionally substituted C₂-C₂₄ alkenyl, optionally substituted C₅-C₁₂ cycloalkenyl, optionally substituted C₂-C₂₄ alkynyl, optionally substituted C₈-C₁₂ cycloalkynyl, optionally substituted C₆-C₁₂ aryl, optionally substituted C₂-C₁₂ heteroaryl, —C(═O)OH, —C(═O)O(optionally substituted C₁-C₂₄ alkyl), —C(═O)O(optionally substituted C₂-C₂₄ alkenyl), —C(═O)O(optionally substituted C₆-C₁₂ aryl), —C(═O)O(optionally substituted C₂-C₁₂ heteroaryl), amido, amino, —C(═O)(optionally substituted C₁-C₂₄ alkyl), —C(═O)(optionally substituted C₂-C₂₄ alkenyl), —C(═O)(optionally substituted C₆-C₁₂ aryl), and —C(═O)(optionally substituted C₂-C₁₂ heteroaryl);
or R₂₀ and R₂₁ may combine with Y to form a —(C═O)—;
wherein z′ and z″ are each independently an integer represented by 0, 1, or 2; and
wherein m, n, o, p, q, r, s, t, u, v, w, and x are each independently an integer represented by 0, 1, 2; 3, 4, or 5.

In certain embodiments, the ionizable lipid compound or salt thereof is a compound of formula (II):

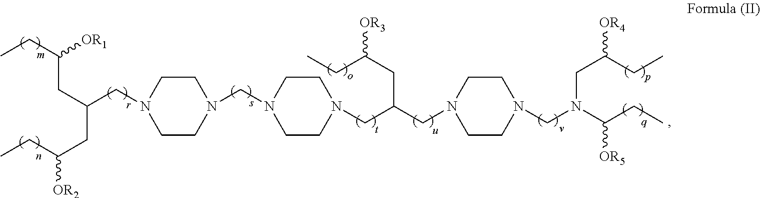

wherein:
R₁, R₂, R₃, R₄, and R₅ are each independently selected from the group consisting of H, optionally substituted C₁-C₂₄ alkyl, optionally substituted C₃-C₁₂ cycloalkyl, optionally substituted C₂-C₁₂ heterocycloalkyl, optionally substituted C₂-C₂₄ alkenyl, optionally substituted C₅-C₁₂ cycloalkenyl, optionally substituted C₂-C₂₄ alkynyl, optionally substituted C₈-C₁₂ cycloalkynyl, optionally substituted C₆-C₁₂ aryl, optionally substituted C₂-C₁₂ heteroaryl, —C(═O)OH, —C(═O)(optionally substituted C₁-C₂₄ alkyl), —C(═O)(optionally substituted C₂-C₂₄ alkenyl), —C(═O)(optionally substituted C₆-C₁₂ aryl), and —C(═O)(optionally substituted C₂-C₁₂ heteroaryl);
m, n, n, o, p, and q are each independently an integer ranging from 0 to 24; and
r, s, t, u, and v are each independently an integer selected from the group consisting of 0, 1, 2, 3, 4, and 5.

In certain embodiments, the ionizable lipid compound or salt thereof is a compound of formula (III):

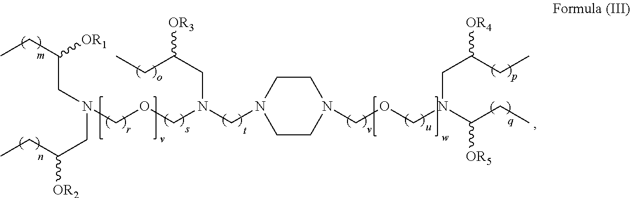

wherein:
R₁, R₂, R₃, R₄, and R₅ are each independently selected from the group consisting of H, optionally substituted C₁-C₂₄ alkyl, optionally substituted C₃-C₁₂ cycloalkyl, optionally substituted C₂-C₁₂ heterocycloalkyl, optionally substituted C₂-C₂₄ alkenyl, optionally substituted C₅-C₁₂ cycloalkenyl, optionally substituted C₂-C₂₄ alkynyl, optionally substituted C₈-C₁₂ cycloalkynyl, optionally substituted C₆-C₁₂ aryl, optionally substituted C₂-C₁₂ heteroaryl, —C(═O)OH, —C(═O)(optionally substituted C₁-C₂₄ alkyl), —C(═O)(optionally substituted C₂-C₂₄ alkenyl), —C(═O)(optionally substituted C₆-C₁₂ aryl), and —C(═O)(optionally substituted C₂-C₁₂ heteroaryl);
m, n, o, p, and q are each independently an integer ranging from 0 to 24; and
each occurrence of r, s, t, u, v, and w is independently an integer selected from the group consisting of 0, 1, 2, 3, 4, and 5.

In certain embodiments, the ionizable lipid compound or salt thereof is a compound of formula (IV):

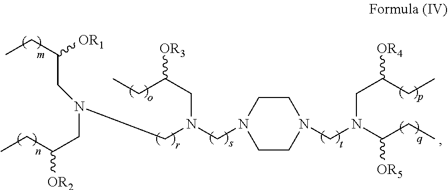

wherein:
R₁, R₂, R₃, R₄, and R₅ are each independently selected from the group consisting of H, optionally substituted C₁-C₂₄ alkyl, optionally substituted C₃-C₁₂ cycloalkyl, optionally substituted C₂-C₁₂ heterocycloalkyl, optionally substituted C₂-C₂₄ alkenyl, optionally substituted C₅-C₁₂ cycloalkenyl, optionally substituted C₂-C₂₄ alkynyl, optionally substituted C₈-C₁₂ cycloalkynyl, optionally substituted C₆-C₁₂ aryl, optionally substituted C₂-C₁₂ heteroaryl, —C(═O)OH, —C(═O)(optionally substituted C₁-C₂₄ alkyl), —C(═O)(optionally substituted C₂-C₂₄ alkenyl), —C(═O)(optionally substituted C₆-C₁₂ aryl), and —C(═O)(optionally substituted C₂-C₁₂ heteroaryl);
m, n, o, p, and q are each independently an integer ranging from 0 to 24; and
r, s, and t are each independently an integer selected from the group consisting of 0, 1, 2, 3, 4, and 5.

In certain embodiments, the ionizable lipid compound or salt thereof is a compound of formula (V):

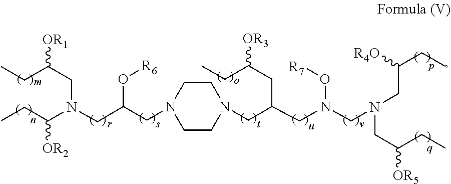

wherein:
R₁, R₂, R₃, R₄, R₅, R₆, and R₇ are each independently selected from the group consisting of H, optionally substituted C₁-C₂₄ alkyl, optionally substituted C₃-C₁₂ cycloalkyl, optionally substituted C₂-C₁₂ heterocycloalkyl, optionally substituted C₂-C₂₄ alkenyl, optionally substituted C₅-C₁₂ cycloalkenyl, optionally substituted C₂-C₂₄ alkynyl, optionally substituted C₈-C₁₂ cycloalkynyl, optionally substituted C₆-C₁₂ aryl, optionally substituted C₂-C₁₂ heteroaryl, —C(═O)OH, —C(═O)(optionally substituted C₁-C₂₄ alkyl), —C(═O)(optionally substituted C₂-C₂₄ alkenyl), —C(═O)(optionally substituted C₆-C₁₂ aryl), and —C(═O)(optionally substituted C₂-C₁₂ heteroaryl);
m, n, o, p, and q are each independently an integer ranging from 0 to 24; and
r, s, t, u, and v are each independently an integer selected from the group consisting of 0, 1, 2, 3, 4, and 5.

In certain embodiments, the ionizable lipid compound or salt thereof is a compound of formula (VI):

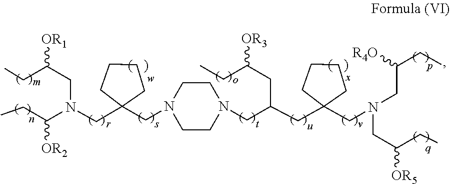

wherein:
R₁, R₂, R₃, R₄, and R₅ are each independently selected from the group consisting of H, optionally substituted C₁-C₂₄ alkyl, optionally substituted C₃-C₁₂ cycloalkyl, optionally substituted C₂-C₁₂ heterocycloalkyl, optionally substituted C₂-C₂₄ alkenyl, optionally substituted C₅-C₁₂ cycloalkenyl, optionally substituted C₂-C₂₄ alkynyl, optionally substituted C₈-C₁₂ cycloalkynyl, optionally substituted C₆-C₁₂ aryl, optionally substituted C₂-C₁₂ heteroaryl, —C(═O)OH, —C(═O)(optionally substituted C₁-C₂₄ alkyl), —C(═O)(optionally substituted C₂-C₂₄ alkenyl), —C(═O)(optionally substituted C₆-C₁₂ aryl), and —C(═O)(optionally substituted C₂-C₁₂ heteroaryl);
m, n, o, p, and q are each independently an integer ranging from 0 to 24;
r, s, t, u, and v are each independently selected from the group consisting of 0, 1, 2, 3, 4, and 5; and
w and x are each independently an integer selected from the group consisting of 1, 2, 3, and 4.

In certain embodiments, the ionizable lipid compound or salt thereof is a compound of formula (VII):

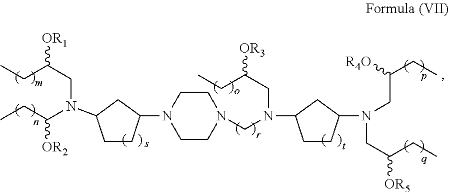

wherein:
R₁, R₂, R₃, R₄, and R₅ are each independently selected from the group consisting of H, optionally substituted C₁-C₂₄ alkyl, optionally substituted C₃-C₁₂ cycloalkyl, optionally substituted C₂-C₁₂ heterocycloalkyl, optionally substituted C₂-C₂₄ alkenyl, optionally substituted C₅-C₁₂ cycloalkenyl, optionally substituted C₂-C₂₄ alkynyl, optionally substituted C₈-C₁₂ cycloalkynyl, optionally substituted C₆-C₁₂ aryl, optionally substituted C₂-C₁₂ heteroaryl, —C(═O)OH, —C(═O)(optionally substituted C₁-C₂₄ alkyl), —C(═O)(optionally substituted C₂-C₂₄ alkenyl), —C(═O)(optionally substituted C₆-C₁₂ aryl), and —C(═O)(optionally substituted C₂-C₁₂ heteroaryl);
m, n, o, p, and q are each independently an integer ranging from 0 to 24; and
r is an integer selected from the group consisting of 1, 2, 3, 4, and 5;
s and t are each independently an integer selected from the group consisting of 0, 1, 2, 3, 4, and 5.

In certain embodiments, the ionizable lipid compound or salt thereof is a compound of formula (VIII):

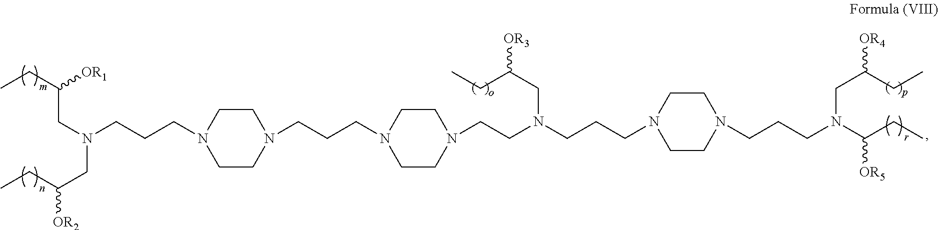

wherein:
R₁, R₂, R₃, R₄, and R₅ are each independently selected from the group consisting of H, optionally substituted C₁-C₂₄ alkyl, optionally substituted C₃-C₁₂ cycloalkyl, optionally substituted C₂-C₁₂ heterocycloalkyl, optionally substituted C₂-C₂₄ alkenyl, optionally substituted C₅-C₁₂ cycloalkenyl, optionally substituted C₂-C₂₄ alkynyl, optionally substituted C₈-C₁₂ cycloalkynyl, optionally substituted C₆-C₁₂ aryl, optionally substituted C₂-C₁₂ heteroaryl, —C(═O)OH, —C(═O)(optionally substituted C₁-C₂₄ alkyl), —C(═O)(optionally substituted C₂-C₂₄ alkenyl), —C(═O)(optionally substituted C₆-C₁₂ aryl), and —C(═O)(optionally substituted C₂-C₁₂ heteroaryl); and
m, n, o, p, and r are each independently an integer ranging from 0 to 24.

In certain embodiments, the ionizable lipid compound or salt thereof is a compound of formula (IX):

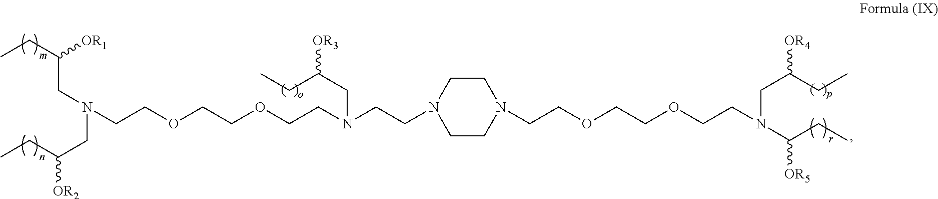

wherein:
R₁, R₂, R₃, R₄, and R₅ are each independently selected from the group consisting of H, optionally substituted C₁-C₂₄ alkyl, optionally substituted C₃-C₁₂ cycloalkyl, optionally substituted C₂-C₁₂ heterocycloalkyl, optionally substituted C₂-C₂₄ alkenyl, optionally substituted C₅-C₁₂ cycloalkenyl, optionally substituted C₂-C₂₄ alkynyl, optionally substituted C₅-C₁₂ cycloalkynyl, optionally substituted C₆-C₁₂ aryl, optionally substituted C₂-C₁₂ heteroaryl, —C(═O)OH, —C(═O)(optionally substituted C₁-C₂₄ alkyl), —C(═O)(optionally substituted C₂-C₂₄ alkenyl), —C(═O)(optionally substituted C₆-C₁₂ aryl), and —C(═O)(optionally substituted C₂-C₁₂ heteroaryl); and
m, n, o, p, and r are each independently an integer ranging from 0 to 24.

In certain embodiments, the ionizable lipid compound or salt thereof is a compound of formula (X):

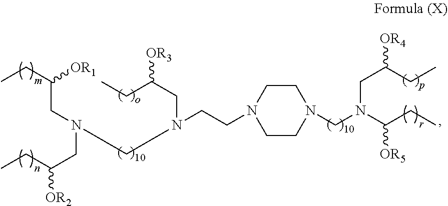

wherein:
R₁, R₂, R₃, R₄, and R₅ are each independently selected from the group consisting of H, optionally substituted C₁-C₂₄ alkyl, optionally substituted C₃-C₁₂ cycloalkyl, optionally substituted C₂-C₁₂ heterocycloalkyl, optionally substituted C₂-C₂₄ alkenyl, optionally substituted C₅-C₁₂ cycloalkenyl, optionally substituted C₂-C₂₄ alkynyl, optionally substituted C₅-C₁₂ cycloalkynyl, optionally substituted C₆-C₁₂ aryl, optionally substituted C₂-C₁₂ heteroaryl, —C(═O)OH, —C(═O)(optionally substituted C₁-C₂₄ alkyl), —C(═O)(optionally substituted C₂-C₂₄ alkenyl), —C(═O)(optionally substituted C₆-C₁₂ aryl), and —C(═O)(optionally substituted C₂-C₁₂ heteroaryl); and
m, n, o, p, and r are each independently an integer ranging from 0 to 24.

In certain embodiments, the ionizable lipid compound or salt thereof is a compound of formula (XI):

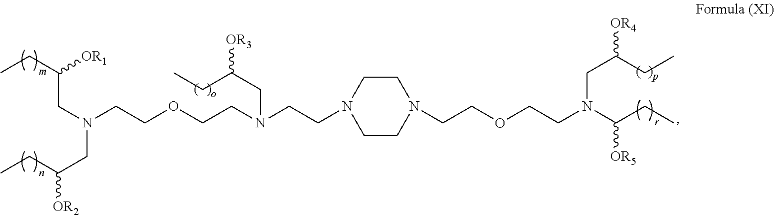

wherein:
R₁, R₂, R₃, R₄, and R₅ are each independently selected from the group consisting of H, optionally substituted C₁-C₂₄ alkyl, optionally substituted C₃-C₁₂ cycloalkyl, optionally substituted C₂-C₁₂ heterocycloalkyl, optionally substituted C₂-C₂₄ alkenyl, optionally substituted C₅-C₁₂ cycloalkenyl, optionally substituted C₂-C₂₄ alkynyl, optionally substituted C₈-C₁₂ cycloalkynyl, optionally substituted C₆-C₁₂ aryl, optionally substituted C₂-C₁₂ heteroaryl, —C(═O)OH, —C(═O)(optionally substituted C₁-C₂₄ alkyl), —C(═O)(optionally substituted C₂-C₂₄ alkenyl), —C(═O)(optionally substituted C₆-C₁₂ aryl), and —C(═O)(optionally substituted C₂-C₁₂ heteroaryl); and
m, n, o, p, and r are each independently an integer ranging from 0 to 24.

In certain embodiments, the ionizable lipid compound or salt thereof is a compound of formula (XII):

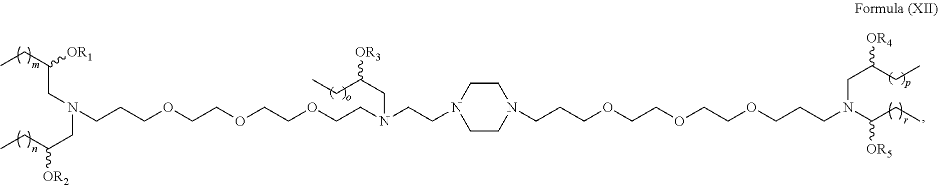

wherein:
R₁, R₂, R₃, R₄, and R₅ are each independently selected from the group consisting of H, optionally substituted C₁-C₂₄ alkyl, optionally substituted C₃-C₁₂ cycloalkyl, optionally substituted C₂-C₁₂ heterocycloalkyl, optionally substituted C₂-C₂₄ alkenyl, optionally substituted C₅-C₁₂ cycloalkenyl, optionally substituted C₂-C₂₄ alkynyl, optionally substituted C₅-C₁₂ cycloalkynyl, optionally substituted C₆-C₁₂ aryl, optionally substituted C₂-C₁₂ heteroaryl, —C(═O)OH, —C(═O)(optionally substituted C₁-C₂₄ alkyl), —C(═O)(optionally substituted C₂-C₂₄ alkenyl), —C(═O)(optionally substituted C₆-C₁₂ aryl), and —C(═O)(optionally substituted C₂-C₁₂ heteroaryl); and
m, n, o, p, and r are each independently an integer ranging from 0 to 24.

In certain embodiments, the ionizable lipid compound or salt thereof is a compound of formula (XIII):

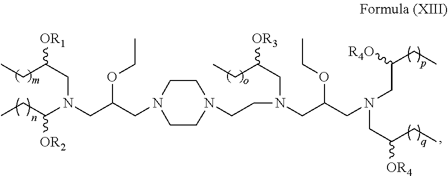

wherein:
R₁, R₂, R₃, R₄, and R₅ are each independently selected from the group consisting of H, optionally substituted C₁-C₂₄ alkyl, optionally substituted C₃-C₁₂ cycloalkyl, optionally substituted C₂-C₁₂ heterocycloalkyl, optionally substituted C₂-C₂₄ alkenyl, optionally substituted C₅-C₁₂ cycloalkenyl, optionally substituted C₂-C₂₄ alkynyl, optionally substituted C₈-C₁₂ cycloalkynyl, optionally substituted C₆-C₁₂ aryl, optionally substituted C₂-C₁₂ heteroaryl, —C(═O)OH, —C(═O)(optionally substituted C₁-C₂₄ alkyl), —C(═O)(optionally substituted C₂-C₂₄ alkenyl), —C(═O)(optionally substituted C₆-C₁₂ aryl), and —C(═O)(optionally substituted C₂-C₁₂ heteroaryl); and
m, n, o, p, and q are each independently an integer ranging from 0 to 24.

In certain embodiments, the ionizable lipid compound or salt thereof is a compound of formula (XIV):

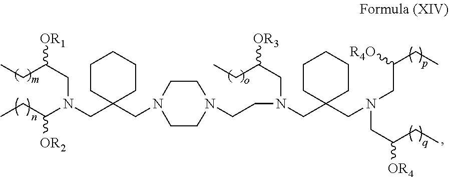

wherein:
R₁, R₂, R₃, R₄, and R₅ are each independently selected from the group consisting of H, optionally substituted C₁-C₂₄ alkyl, optionally substituted C₃-C₁₂ cycloalkyl, optionally substituted C₂-C₁₂ heterocycloalkyl, optionally substituted C₂-C₂₄ alkenyl, optionally substituted C₅-C₁₂ cycloalkenyl, optionally substituted C₂-C₂₄ alkynyl, optionally substituted C₅-C₁₂ cycloalkynyl, optionally substituted C₆-C₁₂ aryl, optionally substituted C₂-C₁₂ heteroaryl, —C(═O)OH, —C(═O)(optionally substituted C₁-C₂₄ alkyl), —C(═O)(optionally substituted C₂-C₂₄ alkenyl), —C(═O)(optionally substituted C₆-C₁₂ aryl), and —C(═O)(optionally substituted C₂-C₁₂ heteroaryl); and
m, n, o, p, and q are each independently an integer ranging from 0 to 24.

In certain embodiments, the ionizable lipid compound or salt thereof is a compound of formula (XV):

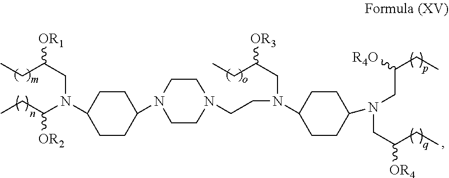

wherein:
R₁, R₂, R₃, R₄, and R₅ are each independently selected from the group consisting of H, optionally substituted C₁-C₂₄ alkyl, optionally substituted C₃-C₁₂ cycloalkyl, optionally substituted C₂-C₁₂ heterocycloalkyl, optionally substituted C₂-C₂₄ alkenyl, optionally substituted C₅-C₁₂ cycloalkenyl, optionally substituted C₂-C₂₄ alkynyl, optionally substituted C₈-C₁₂ cycloalkynyl, optionally substituted C₆-C₁₂ aryl, optionally substituted C₂-C₁₂ heteroaryl, —C(═O)OH, —C(═O)(optionally substituted C₁-C₂₄ alkyl), —C(═O)(optionally substituted C₂-C₂₄ alkenyl), —C(═O)(optionally substituted C₆-C₁₂ aryl), and —C(═O)(optionally substituted C₂-C₁₂ heteroaryl); and
m, n, o, p, and q are each independently an integer ranging from 0 to 24.

In certain embodiments, the ionizable lipid compound or salt thereof comprises 1,1′-((2-(2-(4-(2-((2-(2-(bis(2-hydroxytetradecyl)amino)ethoxy)ethyl)(2-hydroxytetradecyl)amino)ethyl)piperazin-1-yl)ethoxy)ethyl)azanediyl)bis(tetradecan-2-ol), herein referred to as “B-4” and comprising a structure of Formula (XVI):

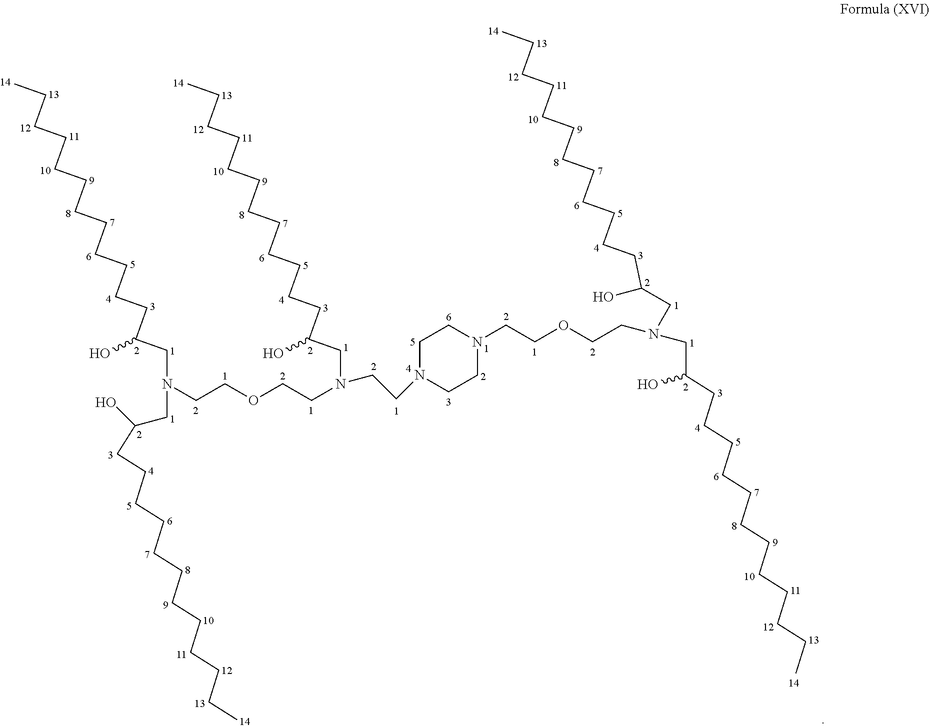 [SMILES: CCCCCCCCCCCCC(O)CN(CCOCCN(CC(O)CCCCCCCCCCCC)CC(O)CCCCCCCCCCCC)CCN1CCN(CCOCCN(CC(O)CCCCCCCCCCCC)CC(O)CCCCCCCCCCCC)CC1]

In certain embodiments, in any of the compounds of Formula (I)-Formula (XVI) each occurrence of optionally substituted alkyl, optionally substituted cycloalkyl, optionally substituted heterocycloalkyl, optionally substituted alkenyl, optionally substituted cycloalkenyl, optionally substituted alkynyl, optionally substituted cycloalkynyl, optionally substituted aryl, and optionally substituted heteroaryl, if present, is independently optionally substituted with at least one substituent selected from the group consisting of C₁-C₆ alkyl, C₃-C₈ cycloalkyl, C₁-C₆ haloalkyl, C₁-C₃ haloalkoxy, phenoxy, halogen, CN, NO₂, OH, N(R′)(R″), C(═O)R′, C(═O)OR′, OC(═O)OR′, C(═O)N(R′)(R″), S(═O)₂N(R′)(R″), N(R′)C(═O)R″, N(R′)S(═O)₂R″, C₂-C₈ heteroaryl, and phenyl optionally substituted with at least one halogen, wherein each occurrence of R′ and R″ is independently selected from the group consisting of H, C₁-C₆ alkyl, C₃-C₈ cycloalkyl, C₁-C₆ haloalkyl, benzyl, and phenyl.

In certain embodiments, R₁ is H. In certain embodiments, R₂ is H. In certain embodiments, R₁₇ is H. In certain embodiments, R₁₈ is H. In certain embodiments R₁₉ is H.
In certain embodiments, R₁ is —CH₂—CH(OH)—(C₁-C₂₂ alkyl). In certain embodiments, R₂ is —CH₂—CH(OH)—(C₁-C₂₂ alkyl). In certain embodiments, R₁₇ is —CH₂—CH(OH)—(C₁-C₂₂ alkyl). In certain embodiments, R₁₈ is —CH₂—CH(OH)—(C₁-C₂₂ alkyl). In certain embodiments R₁₉ is —CH₂—CH(OH)—(C₁-C₂₂ alkyl).

In certain embodiments, R₁ is —CH₂—CH(OH)—(CH₂)₁₁CH₃. In certain embodiments, R₂ is —CH₂—CH(OH)—(CH₂)₁₁CH₃. In certain embodiments, R₁₇ is —CH₂—CH(OH)—(CH₂)₁₁CH₃. In certain embodiments, R₁₈ is —CH₂—CH(OH)—(CH₂)₁₁CH₃. In certain embodiments R₁₉ is —CH₂—CH(OH)—(CH₂)₁₁CH₃.

In certain embodiments, the ionizable lipid comprises:

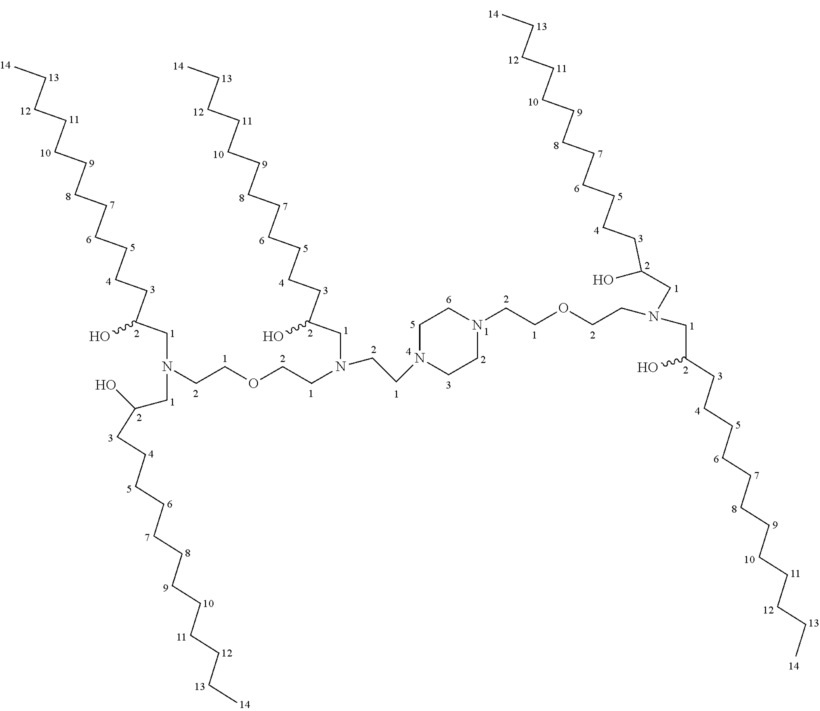 [SMILES: CCCCCCCCCCCCC(O)CN(CCOCCN(CC(O)CCCCCCCCCCCC)CC(O)CCCCCCCCCCCC)CCN1CCN(CCOCCN(CC(O)CCCCCCCCCCCC)CC(O)CCCCCCCCCCCC)CC1]

In certain embodiments, the molar ratio of (a):(b):(c):(d) is about 15:10:5:0.5. In certain embodiments, the molar ratio of (a):(b):(c):(d) is about 15:20:20:4.5. In certain embodiments, the molar ratio of (a):(b):(c):(d) is about 15:40:50:12.5. In certain embodiments, the molar ratio of (a):(b):(c):(d) is about 35:30:5:4.5. In certain embodiments, the molar ratio of (a):(b):(c):(d) in the compound of formula (I) is about 35:40:20:0.5. In certain embodiments, the molar ratio of (a):(b):(c):(d) in the compound of formula (I) is about 45:20:35:0.5. In certain embodiments, the molar ratio of (a):(b):(c):(d) in the compound of formula (I) is about 45:40:5:8.5.

In certain embodiments, the LNP further comprises at least one selected from the group consisting of a nucleic acid molecule, therapeutic agent, and any combination thereof. In certain embodiments, the nucleic acid molecule is a therapeutic agent. In certain embodiments, the nucleic acid molecule is a DNA molecule or an RNA molecule. In certain embodiments, the nucleic acid molecule is selected from the group consisting of cDNA, mRNA, miRNA, siRNA, modified RNA, antagomir, antisense molecule, peptide, therapeutic peptide, targeted nucleic acid, and any combination thereof. In certain embodiments, the nucleic acid molecule encodes one or more components for gene editing. In certain embodiments, the mRNA encodes one or more antigens.

In various embodiments, the LNP comprises one or more ionizable lipid compound of the present disclosure in a concentration range of about 0.1 mol % to about 100 mol %. In some embodiments, the LNP comprises one or more lipids of the present disclosure in a concentration range of about 1 mol % to about 100 mol %. In some embodiments, the LNP comprises one or more lipids of the present disclosure in a concentration range of about 10 mol % to about 70 mol %. In some embodiments, the LNP comprises one or more lipids of the present disclosure in a concentration range of about 10 mol % to about 50 mol %. In some embodiments, the LNP comprises one or more lipids of the present disclosure in a concentration range of about 15 mol % to about 45 mol %. In some embodiments, the LNP comprises one or more lipids of the present disclosure in a concentration range of about 35 mol % to about 40 mol %.

For example, in some embodiments, the LNP comprises one or more lipids of the present disclosure in a concentration of about 1 mol %. In some embodiments, the LNP comprises one or more lipids of the present disclosure in a concentration of about 2 mol %. In some embodiments, the LNP comprises one or more lipids of the present disclosure in a concentration of about 5 mol %. In some embodiments, the LNP comprises one or more lipids of the present disclosure in a concentration of about 5.5 mol %. In some embodiments, the LNP comprises one or more lipids of the present disclosure in a concentration of about 10 mol %. In some embodiments, the LNP comprises one or more lipids of the present disclosure in a concentration of about 12 mol %. In some embodiments, the LNP comprises one or more lipids of the present disclosure in a concentration of about 15 mol %. In some embodiments, the LNP comprises one or more lipids of the present disclosure in a concentration of about 20 mol %. In some embodiments, the LNP comprises one or more lipids of the present disclosure in a concentration of about 25 mol %. In some embodiments, the LNP comprises one or more lipids of the present disclosure in a concentration of about 30 mol %. In some embodiments, the LNP comprises one or more lipids of the present disclosure in a concentration of about 35 mol %. In some embodiments, the LNP comprises one or more lipids of the present disclosure in a concentration of about 37 mol %. In some embodiments, the LNP comprises one or more lipids of the present disclosure in a concentration of about 40 mol %. In some embodiments, the LNP comprises one or more lipids of the present disclosure in a concentration of about 45 mol %. In some embodiments, the LNP comprises one or more lipids of the present disclosure in a concentration of about 50 mol %. In some embodiments, the LNP comprises one or more lipids of the present disclosure in a concentration of about 60 mol %. In some embodiments, the LNP comprises one or more lipids of the present disclosure in a concentration of about 70 mol %. In some embodiments, the LNP comprises one or more lipids of the present disclosure in a concentration of about 80 mol %. In some embodiments, the LNP comprises one or more lipids of the present disclosure in a concentration of about 90 mol %. In some embodiments, the LNP comprises one or more lipids of the present disclosure in a concentration of about 95 mol %. In some embodiments, the LNP comprises one or more lipids of the present disclosure in a concentration of about 95.5 mol %. In some embodiments, the LNP comprises one or more lipids of the present disclosure in a concentration of about 99 mol %. In some embodiments, the LNP comprises one or more lipids of the present disclosure in a concentration of about 99.9 mol %. In some embodiments, the LNP comprises one or more lipids of the present disclosure in a concentration of about 100 mol %.

In various embodiments, the LNP further comprises at least one helper compound. In some embodiments, the helper compound is a helper lipid, helper polymer, or any combination thereof. In some embodiments, the helper lipid is phospholipid, cholesterol lipid, polymer, cationic lipid, neutral lipid, charged lipid, steroid, steroid analogue, polymer conjugated lipid, stabilizing lipid, or any combination thereof.

In various embodiments, the LNP comprises one or more helper compound in a concentration range of about 0 mol % to about 100 mol %. In some embodiments, the LNP comprises one or more helper compound in a concentration range of about 0.01 mol % to about 99.9 mol %. In some embodiments, the LNP comprises one or more helper compound in a concentration range of about 0.1 mol % to about 90 mol %. In some embodiments, the LNP comprises one or more helper compound in a concentration range of about 0.1 mol % to about 70 mol %. In some embodiments, the LNP comprises one or more helper compound in a concentration range of about 5 mol % to about 95 mol %. In some embodiments, the LNP comprises one or more helper compound in a concentration range of about 0.5 mol % to about 50 mol %. In some embodiments, the LNP comprises one or more helper compound in a concentration range of about 0.5 mol % to about 47 mol %. In some embodiments, the LNP comprises one or more helper compound in a concentration range of about 2.5 mol % to about 47 mol %.

For example, in some embodiments, the LNP comprises one or more helper compound in a concentration of about 0.01 mol %. In some embodiments, the LNP comprises one or more helper compound in a concentration of about 0.1 mol %. In some embodiments, the LNP comprises one or more helper compound in a concentration of about 0.5 mol %. In some embodiments, the LNP comprises one or more helper compound in a concentration of about 1 mol %. In some embodiments, the LNP comprises one or more helper compound in a concentration of about 1.5 mol %. In some embodiments, the LNP comprises one or more helper compound in a concentration of about 2 mol %. In some embodiments, the LNP comprises one or more helper compound in a concentration of about 2.5 mol %. In some embodiments, the LNP comprises one or more helper compound in a concentration of about 5 mol %. In some embodiments, the LNP comprises one or more helper compound in a concentration of about 10 mol %. In some embodiments, the LNP comprises one or more helper compound in a concentration of about 12 mol %. In some embodiments, the LNP comprises one or more helper compound in a concentration of about 15 mol %. In some embodiments, the LNP comprises one or more helper compound in a concentration of about 16 mol %. In some embodiments, the LNP comprises one or more helper compound in a concentration of about 20 mol %. In some embodiments, the LNP comprises one or more helper compound in a concentration of about 25 mol %. In some embodiments, the LNP comprises one or more helper compound in a concentration of about 30 mol %. In some embodiments, the LNP comprises one or more helper compound in a concentration of about 35 mol %. In some embodiments, the LNP comprises one or more helper compound in a concentration of about 37 mol %. In some embodiments, the LNP comprises one or more helper compound in a concentration of about 40 mol %. In some embodiments, the LNP comprises one or more helper compound in a concentration of about 45 mol %. In some embodiments, the LNP comprises one or more helper compound in a concentration of about 46.5 mol %. In some embodiments, the LNP comprises one or more helper compound in a concentration of about 47 mol %. In some embodiments, the LNP comprises one or more helper compound in a concentration of about 50 mol %. In some embodiments, the LNP comprises one or more helper compound in a concentration of about 60 mol %. In some embodiments, the LNP comprises one or more helper compound in a concentration of about 63 mol %. In some embodiments, the LNP comprises one or more helper compound in a concentration of about 70 mol %. In some embodiments, the LNP comprises one or more helper compound in a concentration of about 80 mol %. In some embodiments, the LNP comprises one or more helper compound in a concentration of about 90 mol %. In some embodiments, the LNP comprises one or more helper compound in a concentration of about 95 mol %. In some embodiments, the LNP comprises one or more helper compound in a concentration of about 95.5 mol %. In some embodiments, the LNP comprises one or more helper compound in a concentration of about 99 mol %. In some embodiments, the LNP comprises one or more helper compound in a concentration of about 100 mol %.

In some embodiments, the phospholipid is dioleoyl-phosphatidylethanolamine (DOPE) or a derivative thereof, distearoylphosphatidylcholine (DSPC) or a derivative thereof, distearoyl-phosphatidylethanolamine (DSPE) or a derivative thereof, stearoyloleoylphosphatidylcholine (SOPC) or a derivative thereof, 1-stearioyl-2-oleoyl-phosphatidyethanol amine (SOPE) or a derivative thereof, N-(2,3-dioleoyloxy)propyl)-N,N,N-trimethylammonium chloride (DOTAP) or a derivative thereof, or any combination thereof.

For example, in some embodiments, the LNP comprises a phospholipid in a concentration range of about 0 mol % to about 100 mol %. In some embodiments, the LNP comprises a phospholipid in a concentration range of about 15 mol % to about 50 mol %. In some embodiments, the LNP comprises a phospholipid in a concentration range of about 10 mol % to about 40 mol %. In some embodiments, the LNP comprises a phospholipid in a concentration range of about 16 mol % to about 40 mol %.

In some embodiments, the cholesterol lipid is cholesterol or a derivative thereof. For example, in some embodiments, the LNP comprises a cholesterol lipid in a concentration range of about 0 mol % to about 100 mol %. In some embodiments, the LNP comprises a cholesterol lipid in a concentration range of about 20 mol % to about 50 mol %. In some embodiments, the LNP comprises a cholesterol lipid in a concentration range of about 20 mol % to about 47 mol %. In some embodiments, the LNP comprises a cholesterol lipid in a concentration of about 47 mol % and DOPE in a concentration of about 16 mol %.

In some embodiments, the polymer is polyethylene glycol (PEG) or a derivative thereof. For example, in some embodiments, the LNP comprises a polymer in a concentration range of about 0 mol % to about 100 mol %. In some embodiments, the LNP comprises a polymer in a concentration range of about 0.5 mol % to about 10 mol %. In some embodiments, the LNP comprises a polymer in a concentration range of about 0.5 mol % to about 2.5 mol %.

As used herein, the term “cationic lipid” refers to a lipid that is cationic or becomes cationic (protonated) as the pH is lowered below the pK of the ionizable group of the lipid, but is progressively more neutral at higher pH values. At pH values below the pK, the lipid is then able to associate with negatively charged nucleic acids. In certain embodiments, the cationic lipid comprises a zwitterionic lipid that assumes a positive charge on pH decrease.

In some embodiments, the cationic lipid comprises any of a number of lipid species which carry a net positive charge at a selective pH, such as physiological pH. Such lipids include, but are not limited to, N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3-dioleoyloxy)propyl)-N,N,N-trimethylammonium chloride (DOTAP); 3-(N—(N′,N′-dimethylaminoethane)-carbamoyl)cholesterol (DC-Chol), N-(1-(2,3-dioleoyloxy)propyl)-N-2-(sperminecarboxamido)ethyl)-N,N-dimethylammonium trifluoracetate (DOSPA), dioctadecylamidoglycyl carboxyspermine (DOGS), 1,2-dioleoyl-3-dimethylammonium propane (DODAP), N,N-dimethyl-2,3-dioleoyloxy)propylamine (DODMA), and N-(1,2-dimyristyloxyprop-3-yl)-N,N-dimethyl-N-hydroxyethyl ammonium bromide (DMRIE). Additionally, a number of commercial preparations of cationic lipids are available which can be used in the present disclosure. These include, for example, LIPOFECTIN® (commercially available cationic liposomes comprising DOTMA and 1,2-dioleoyl-sn-3-phosphoethanolamine (DOPE), from GIBCO/BRL, Grand Island, N.Y.); LIPOFECTAMINE® (commercially available cationic liposomes comprising N-(1-(2,3-dioleyloxy)propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.). The following lipids are cationic and have a positive charge at below physiological pH. DODAP, DODMA, DMDMA, 1,2-dilinoleyloxy-N,N-dimethylaminopropane (DLinDMA), 1,2-dilinolenyloxy-N,N-dimethylaminopropane (DLenDMA).

In certain embodiments, the cationic lipid is an amino lipid. Suitable amino lipids useful in the disclosure include those described in WO 2012/016184, incorporated herein by reference in its entirety. Representative amino lipids include, but are not limited to, 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA).

In certain embodiments, the lipid is a PEGylated lipid, including, but not limited to, DSPE-PEG-DBCO, DOPE-PEG-Azide, DSPE-PEG-Azide, DPPE-PEG-Azide, DSPE-PEG-Carboxy-NHS, DOPE-PEG-Carboxylic Acid, DSPE-PEG-Carboxylic acid.

The term “neutral lipid” refers to any one of a number of lipid species that exist in either an uncharged or neutral zwitterionic form at physiological pH. Representative neutral lipids include diacylphosphatidylcholines, diacylphosphatidylethanolamines, ceramides, sphingomyelins, dihydro sphingomyelins, cephalins, and cerebrosides.

Exemplary neutral lipids include, for example, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cy clohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), distearoyl-phosphatidylethanolamine (DSPE)-maleimide-PEG, distearoyl-phosphatidylethanolamine (DSPE)-maleimide-PEG2000, 16-O-monomethyl PE, 16-O-dimethyl PE, 18-1-trans PE, 1-stearioyl-2-oleoyl-phosphatidyethanol amine (SOPE), stearoyloleoylphosphatidylcholine (SOPC), and 1,2-dielaidoyl-sn-glycero-3-phophoethanolamine (transDOPE). In certain embodiments, the neutral lipid is 1,2-distearoyl-sn-glycero-3-phosphocholine (DSPC).

In some embodiments, the composition comprises a neutral lipid selected from DSPC, DPPC, DMPC, DOPC, POPC, DOPE, and SM.

A “steroid” is a compound comprising the following carbon skeleton:

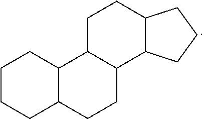 [SMILES: C1CCC2C(C1)CCC1C3CCCC3CCC21]

In certain embodiments, the steroid or steroid analogue is cholesterol. In some of these embodiments, the molar ratio of the cationic lipid.

The term “anionic lipid” refers to any lipid that is negatively charged at physiological pH. These lipids include phosphatidylglycerol, cardiolipin, diacylphosphatidylserine, diacylphosphatidic acid, N-dodecanoylphosphatidylethanolamines, N-succinylphosphatidylethanolamines, N-glutarylphosphatidylethanolamines, lysylphosphatidylglycerols, palmitoyloleyolphosphatidylglycerol (POPG), and other anionic modifying groups joined to neutral lipids.

The term “polymer conjugated lipid” refers to a molecule comprising both a lipid portion and a polymer portion. An example of a polymer conjugated lipid is a pegylated lipid.

The term “pegylated lipid” refers to a molecule comprising both a lipid portion and a polyethylene glycol portion. Pegylated lipids are known in the art and include 1-(monomethoxy-polyethyleneglycol)-2,3-dimyristoylglycerol (PEG-s-DMG), DSPE-PEG-DBCO, DOPE-PEG-Azide, DSPE-PEG-Azide, DPPE-PEG-Azide, DSPE-PEG-Carboxy-NHS, DOPE-PEG-Carboxylic Acid, DSPE-PEG-Carboxylic acid and the like.

In certain embodiments, the LNP comprises an additional, stabilizing-lipid which is a polyethylene glycol-lipid (pegylated lipid). Suitable polyethylene glycol-lipids include PEG-modified phosphatidylethanolamine, PEG-modified phosphatidic acid, PEG-modified ceramides (e.g., PEG-CerC14 or PEG-CerC20), PEG-modified dialkylamines, PEG-modified diacylglycerols, PEG-modified dialkylglycerols. Representative polyethylene glycol-lipids include PEG-c-DOMG, PEG-c-DMA, and PEG-s-DMG. In certain embodiments, the polyethylene glycol-lipid is N-[(methoxy poly(ethylene glycol)₂₀₀₀)carbamyl]-1,2-dimyristyloxlpropyl-3-amine (PEG-c-DMA). In certain embodiments, the polyethylene glycol-lipid is PEG-c-DOMG). In other embodiments, the LNPs comprise a pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy)propyl-1-O-(o-methoxy(polyethoxy)ethyl)butanedioate (PEG-S-DMG), a pegylated ceramide (PEG-cer), or a PEG dialkoxypropylcarbamate such as ω-methoxy(polyethoxy)ethyl-N-(2,3-di(tetradecanoxy)propyl)carbamate or 2,3-di(tetradecanoxy)propyl-N-(ω-methoxy(polyethoxy)ethyl)carbamate.

In certain embodiments, the additional lipid is present in the LNP in an amount from about 1 mol % to about 10 mol %. In certain embodiments, the additional lipid is present in the LNP in an amount from about 1 mol % to about 5 mol %. In certain embodiments, the additional lipid is present in the LNP in about 1 mol % or about 2.5 mol %.

The term “lipid nanoparticle” refers to a particle having at least one dimension on the order of nanometers (e.g., 1-1,000 nm) which includes one or more lipids, for example a lipid of Formula (I)-(XVI).

In various embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm.

In various embodiments, the lipids or the LNP of the present disclosure are substantially non-toxic.

In various embodiments, the lipids or the LNPs described herein are formulated for stability for in utero administration.

In some embodiments, the LNP formulated for stability for in utero administration comprises B-4 in a concentration range of about 10 mol % to about 45 mol %. In some embodiments, the B-4 is present in a molar ratio of about 35 or at a molar percentage of about 35%. In certain embodiments, the B-4 is present in a molar ratio of about 45, or at a molar percentage of about 45%.

In certain embodiments, the phospholipid is dioleoyl-phosphatidylethanolamine (DOPE), and the DOPE is present in a molar ratio of about 40 or at a molar percentage of about 40%. In certain embodiments, the DOPE is present in a molar ratio of about 20, or at a molar percentage of about 20%.

In some embodiments, the LNP formulated for stability for in utero administration comprises a phospholipid in a concentration range of about 10 mol % to about 45 mol %. In certain embodiments, the phospholipid is dioleoyl-phosphatidylethanolamine (DOPE), and the DOPE is present in a molar ratio of about 40 or at a molar percentage of about 40%. In certain embodiments, the DOPE is present in a molar ratio of about 20, or at a molar percentage of about 20%.

In some embodiments, the LNP formulated for stability for in utero administration comprises a cholesterol lipid in a concentration range of about 5 mol % to about 50 mol %. In certain embodiments, the cholesterol is present in a molar ratio of about 20, or at a molar percentage of about 21%. In certain embodiments, the cholesterol is present in a molar ratio of about 35, or at a molar percentage of about 35%. In certain embodiments, the cholesterol is present in a molar ratio of about 5, or at a molar percentage of about 5%.

In some embodiments, the LNP formulated for stability for in utero administration comprises PEG in a concentration range of about 0.5 mol % to about 12.5 mol %. In certain embodiments, the PEG is present in a molar ratio of about 0.5, or at a molar percentage of about 0.5%. In certain embodiments, the PEG is present in a molar ratio of about 8.5, or at a molar percentage of about 8.5%.

In certain embodiments, the LNP formulated for stability for in utero administration comprises ionizable lipid B-4, DOPE, cholesterol and PEG, wherein the B-4:DOPE:cholesterol:PEG are present in a molar ratio of about 15:10:5:0.5 or at a molar percentage of about 50%:33%:16%:1.6%.

In certain embodiments, the LNP formulated for stability for in utero administration comprises ionizable lipid B-4, DOPE, cholesterol and PEG, wherein the B-4:DOPE:cholesterol:PEG are present in a molar ratio of about 15:20:20:4.5 or at a molar percentage of about 25%:34%:34%:7.6%.

In certain embodiments, the LNP formulated for stability for in utero administration comprises ionizable lipid B-4, DOPE, cholesterol and PEG, wherein the B-4:DOPE:cholesterol:PEG are present in a molar ratio of about 15:30:35:8.5 or at a molar percentage of about 17%:34%:40%:9.6%.

In certain embodiments, the LNP formulated for stability for in utero administration comprises ionizable lipid B-4, DOPE, cholesterol and PEG, wherein the B-4:DOPE:cholesterol:PEG are present in a molar ratio of about 15:40:50:12.5 or at a molar percentage of about 13%:34%:43%:11%.

In certain embodiments, the LNP formulated for stability for in utero administration comprises ionizable lipid B-4, DOPE, cholesterol and PEG, wherein the B-4:DOPE:cholesterol:PEG are present in a molar ratio of about 25:10:20:8.5 or at a molar percentage of about 39%:16%:32%:13%.

In certain embodiments, the LNP formulated for stability for in utero administration comprises ionizable lipid B-4, DOPE, cholesterol and PEG, wherein the B-4:DOPE:cholesterol:PEG are present in a molar ratio of about 25:20:5:12.5 or at a molar percentage of about 40%:32%:8.0%:20%.

In certain embodiments, the LNP formulated for stability for in utero administration comprises ionizable lipid B-4, DOPE, cholesterol and PEG, wherein the B-4:DOPE:cholesterol:PEG are present in a molar ratio of about 25:30:50:0.5 or at a molar percentage of about 24%:29%:48%:0.5%.

In certain embodiments, the LNP formulated for stability for in utero administration comprises ionizable lipid B-4, DOPE, cholesterol and PEG, wherein the B-4:DOPE:cholesterol:PEG are present in a molar ratio of about 25:40:35:4.5 or at a molar percentage of about 24%:38%:33%:4.3%.

In certain embodiments, the LNP formulated for stability for in utero administration comprises ionizable lipid B-4, DOPE, cholesterol and PEG, wherein the B-4:DOPE:cholesterol:PEG are present in a molar ratio of about 35:10:35:12.5 or at a molar percentage of about 38%:11%:38%:14%.

In certain embodiments, the LNP formulated for stability for in utero administration comprises ionizable lipid B-4, DOPE, cholesterol and PEG, wherein the B-4:DOPE:cholesterol:PEG are present in a molar ratio of about 35:20:50:8.5 or at a molar percentage of about 31%:18%:44%:7.5%.

In certain embodiments, the LNP formulated for stability for in utero administration comprises ionizable lipid B-4, DOPE, cholesterol and PEG, wherein the B-4:DOPE:cholesterol:PEG are present in a molar ratio of about 35:30:5:4.5 or at a molar percentage of about 47%:40%:21%:0.5%.

In certain embodiments, the LNP formulated for stability for in utero administration comprises ionizable lipid B-4, DOPE, cholesterol and PEG, wherein the B-4:DOPE:cholesterol:PEG are present in a molar ratio of about 35:40:20:0.5 or at a molar percentage of about 37%:42%:21%:0.5%.

In certain embodiments, the LNP formulated for stability for in utero administration comprises ionizable lipid B-4, DOPE, cholesterol and PEG, wherein the B-4:DOPE:cholesterol:PEG are present in a molar ratio of about 45:10:50:4.5 or at a molar percentage of about 41%:9.1%:46%:4.1%.

In certain embodiments, the LNP formulated for stability for in utero administration comprises ionizable lipid B-4, DOPE, cholesterol and PEG, wherein the B-4:DOPE:cholesterol:PEG are present in a molar ratio of about 45:20:35:0.5 or at a molar percentage of about 45%:20%:35%:0.5%.

In certain embodiments, the LNP formulated for stability for in utero administration comprises ionizable lipid B-4, DOPE, cholesterol and PEG, wherein the B-4:DOPE:cholesterol:PEG are present in a molar ratio of about 45:30:20:12.5 or at molar percentage of about 42%:28%:19%:12%.

In certain embodiments, the LNP formulated for stability for in utero administration comprises ionizable lipid B-4, DOPE, cholesterol and PEG, wherein the B-4:DOPE:cholesterol:PEG are present in a molar ratio of about 45:40:5:8.5 or at a molar percentage of about 46%:41%:5.1%:8.6%.

### LNP Compositions

In certain embodiments, the composition of the disclosure comprises in vitro transcribed (IVT) RNA molecule. For example, in certain embodiments, the composition of the disclosure comprises IVT RNA molecule which encodes an agent. In certain embodiments, the IVT RNA molecule of the present composition is a nucleoside-modified mRNA molecule. In certain embodiments, the agent is at least one of a viral antigen, bacterial antigen, fungal antigen, parasitic antigen, tumor-specific antigen, or tumor-associated antigen. However, the present disclosure is not limited to any particular agent or combination of agents. In certain embodiments, the composition comprises an adjuvant. In certain embodiments, the composition comprises a nucleic acid molecule encoding an adjuvant. In certain embodiments, the composition comprises a nucleoside-modified RNA encoding an adjuvant. In certain embodiments, the adjuvant is squalene. In certain embodiments, the adjuvant is a TLR7 agonist. In certain embodiments, the TLR7 agonist is an imidazoquinoline (e.g., dactolisib, imiquimod, gardiquimod, resiquimod, and sumanirole). In certain embodiments, the TLR7 agonist is loxoribine. In certain embodiments, the TLR7 agonist is bropirimine. In certain embodiments, the adjuvant is a TLR8 agonist. In certain embodiments, the TLR8 agonist is one or more small synthetic compounds. In certain embodiments, the TLR8 agonist is a single-stranded viral RNA. In certain embodiments, the TLR8 agonist is a phagocytized bacterial RNA.

In certain embodiments, the composition comprises at least one nucleoside-modified RNA molecule encoding a combination of at least two agents. In certain embodiments, the composition comprises a combination of two or more nucleoside-modified RNA molecules encoding a combination of two or more agents.

In certain embodiments, the present disclosure provides a method for inducing an immune response in a subject. For example, the method can be used to provide immunity in the subject against a virus, bacteria, fungus, parasite, cancer, or the like. In some embodiments, the method comprises administering to the subject a composition comprising one or more ionizable LNP molecule formulated for in utero stability comprising one or more nucleoside-modified RNA encoding at least one antigen, an adjuvant, or a combination thereof.

In certain embodiments, the present disclosure provides a method for gene editing of a fetal subject. For example, the method can be used to provide one or more component of a gene editing system (e.g., a component of a CRISPR system) to a fetal subject. In some embodiments, the method comprises administering to the subject a composition comprising one or more ionizable LNP molecule formulated for in utero stability comprising one or more nucleoside-modified RNA molecule for gene editing.

In certain embodiments, the method comprises in utero administration of the composition to a subject. In certain embodiments, the method comprises administering a plurality of doses to the subject. In other embodiments, the method comprises administering a single dose of the composition, where the single dose is effective in delivery of the target therapeutic agent.

### Targeting Domain

In certain embodiments, the composition comprises a targeting domain that directs the delivery vehicle to a site. In certain embodiments, the site is a site in need of the agent comprised within the delivery vehicle. The targeting domain may comprise a nucleic acid, peptide, antibody, small molecule, organic molecule, inorganic molecule, glycan, sugar, hormone, and the like that targets the particle to a site in particular need of the therapeutic agent. In certain embodiments, the particle comprises multivalent targeting, wherein the particle comprises multiple targeting mechanisms described herein. In certain embodiments, the targeting domain of the delivery vehicle specifically binds to a target associated with a site in need of an agent comprised within the delivery vehicle. For example, the targeting domain may be chosen to recognize a ligand that acts as a cell surface marker on target cells associated with a particular disease state. Such a target can be a protein, protein fragment, antigen, or other biomolecule that is associated with the targeted site. In some embodiments, the targeting domain is an affinity ligand which specifically binds to a target. In certain embodiments, the target (e.g. antigen) associated with a site in need of a treatment with an agent. In some embodiments, the targeting domain may be co-polymerized with the composition comprising the delivery vehicle. In some embodiments, the targeting domain may be covalently attached to the composition comprising the delivery vehicle, such as through a chemical reaction between the targeting domain and the composition comprising the delivery vehicle. In some embodiments, the targeting domain is an additive in the delivery vehicle. Targeting domains of the instant disclosure include, but are not limited to, antibodies, antibody fragments, proteins, peptides, and nucleic acids.

In various embodiments, the targeting domain binds to a cell surface molecule of a cell of interest. For example, in various embodiments, the targeting domain binds to a cell surface molecule of an endothelial cell, a stem cell, or an immune cell.

### Peptides

In certain embodiments, the targeting domain of the disclosure comprises a peptide. In certain embodiments, the peptide targeting domain specifically binds to a target of interest.

The peptide of the present disclosure may be made using chemical methods. For example, peptides can be synthesized by solid phase techniques (Roberge J Y et al (1995) Science 269: 202-204), cleaved from the resin, and purified by preparative high performance liquid chromatography. Automated synthesis may be achieved, for example, using the ABI 431 A Peptide Synthesizer (Perkin Elmer) in accordance with the instructions provided by the manufacturer.

The peptide may alternatively be made by recombinant means or by cleavage from a longer polypeptide. The composition of a peptide may be confirmed by amino acid analysis or sequencing.

The variants of the peptides according to the present disclosure may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the peptide is an alternative splice variant of the peptide of the present disclosure, (iv) fragments of the peptides and/or (v) one in which the peptide is fused with another peptide, such as a leader or secretory sequence or a sequence which is employed for purification (for example, His-tag) or for detection (for example, Sv5 epitope tag). The fragments include peptides generated via proteolytic cleavage (including multi-site proteolysis) of an original sequence. Variants may be post-translationally, or chemically modified. Such variants are deemed to be within the scope of those skilled in the art from the teaching herein.

As known in the art the “similarity” between two peptides is determined by comparing the amino acid sequence and its conserved amino acid substitutes of one peptide to a sequence of a second peptide. Variants are defined to include peptide sequences different from the original sequence, preferably different from the original sequence in less than 40% of residues per segment of interest, more preferably different from the original sequence in less than 25% of residues per segment of interest, more preferably different by less than 10% of residues per segment of interest, most preferably different from the original protein sequence in just a few residues per segment of interest and at the same time sufficiently homologous to the original sequence to preserve the functionality of the original sequence. The present disclosure includes amino acid sequences that are at least 60%, 65%, 70%, 72%, 74%, 76%, 78%, 80%, 90%, or 95% similar or identical to the original amino acid sequence. The degree of identity between two peptides is determined using computer algorithms and methods that are widely known for the persons skilled in the art. The identity between two amino acid sequences is preferably determined by using the BLASTP algorithm [BLAST Manual, Altschul, S., et al., NCBI NLM NIH Bethesda, Md. 20894, Altschul, S., et al., J. Mol. Biol. 215: 403-410 (1990)].

The peptides of the disclosure can be post-translationally modified. For example, post-translational modifications that fall within the scope of the present disclosure include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The peptides of the disclosure may include unnatural amino acids formed by post-translational modification or by introducing unnatural amino acids during translation.

### Nucleic Acids

In certain embodiments, the targeting domain of the disclosure comprises an isolated nucleic acid, including for example a DNA oligonucleotide and a RNA oligonucleotide. In certain embodiments, the nucleic acid targeting domain specifically binds to a target of interest. For example, In certain embodiments, the nucleic acid comprises a nucleotide sequence that specifically binds to a target of interest.

The nucleotide sequences of a nucleic acid targeting domain can alternatively comprise sequence variations with respect to the original nucleotide sequences, for example, substitutions, insertions and/or deletions of one or more nucleotides, with the condition that the resulting nucleic acid functions as the original and specifically binds to the target of interest.

In the sense used in this description, a nucleotide sequence is “substantially homologous” to any of the nucleotide sequences describe herein when its nucleotide sequence has a degree of identity with respect to the nucleotide sequence of at least 60%, advantageously of at least 70%, preferably of at least 85%, and more preferably of at least 95%. Other examples of possible modifications include the insertion of one or more nucleotides in the sequence, the addition of one or more nucleotides in any of the ends of the sequence, or the deletion of one or more nucleotides in any end or inside the sequence. The degree of identity between two polynucleotides is determined using computer algorithms and methods that are widely known for the persons skilled in the art. The identity between two amino acid sequences is preferably determined by using the BLASTN algorithm [BLAST Manual, Altschul, S., et al., NCBI NLM NIH Bethesda, Md. 20894, Altschul, S., et al., J. Mol. Biol. 215: 403-410 (1990)].

### Antibodies

In certain embodiments, the targeting domain of the disclosure comprises an antibody, or antibody fragment. In certain embodiments, the antibody targeting domain specifically binds to a target of interest. Such antibodies include polyclonal antibodies, monoclonal antibodies, Fab and single chain Fv (scFv) fragments thereof, bispecific antibodies, heteroconjugates, human and humanized antibodies.

The antibodies may be intact monoclonal or polyclonal antibodies, and immunologically active fragments (e.g., a Fab or (Fab)2 fragment), an antibody heavy chain, an antibody light chain, humanized antibodies, a genetically engineered single chain Fv molecule (Ladner et al, U.S. Pat. No. 4,946,778), or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art.

Such antibodies may be produced in a variety of ways, including hybridoma cultures, recombinant expression in bacteria or mammalian cell cultures, and recombinant expression in transgenic animals. The choice of manufacturing methodology depends on several factors including the antibody structure desired, the importance of carbohydrate moieties on the antibodies, ease of culturing and purification, and cost. Many different antibody structures may be generated using standard expression technology, including full-length antibodies, antibody fragments, such as Fab and Fv fragments, as well as chimeric antibodies comprising components from different species. Antibody fragments of small size, such as Fab and Fv fragments, having no effector functions and limited pharmokinetic activity may be generated in a bacterial expression system. Single chain Fv fragments show low immunogenicity.

### Antigen

The present disclosure provides a composition that induces an immune response in a subject. In certain embodiments, the composition comprises an antigen. In certain embodiments, the composition comprises a nucleic acid sequence which encodes an antigen. For example, in certain embodiments, the composition comprises a nucleoside-modified RNA encoding an antigen. The antigen may be any molecule or compound, including but not limited to a polypeptide, peptide or protein that induces an adaptive immune response in a subject.

In certain embodiments, the antigen comprises a polypeptide or peptide associated with a pathogen, such that the antigen induces an adaptive immune response against the antigen, and therefore the pathogen. In certain embodiments, the antigen comprises a fragment of a polypeptide or peptide associated with a pathogen, such that the antigen induces an adaptive immune response against the pathogen.

In certain embodiments, the antigen comprises an amino acid sequence that is substantially homologous to the amino acid sequence of an antigen described herein and retains the immunogenic function of the original amino acid sequence. For example, in certain embodiments, the amino acid sequence of the antigen has a degree of identity with respect to the original amino acid sequence of at least 60%, advantageously of at least 70%, preferably of at least 85%, and more preferably of at least 95%.

In certain embodiments, the antigen is encoded by a nucleic acid sequence of a nucleic acid molecule. In certain embodiments, the nucleic acid sequence comprises DNA, RNA, cDNA, a variant thereof, a fragment thereof, or a combination thereof. In certain embodiments, the nucleic acid sequence comprises a modified nucleic acid sequence. For example, In certain embodiments the antigen-encoding nucleic acid sequence comprises nucleoside-modified RNA, as described in detail elsewhere herein. In certain instances, the nucleic acid sequence comprises include additional sequences that encode linker or tag sequences that are linked to the antigen by a peptide bond.

In certain embodiments, the antigen, encoded by the nucleoside-modified nucleic acid molecule, comprises a protein, peptide, a fragment thereof, or a variant thereof, or a combination thereof from any number of organisms, for example, a virus, a parasite, a bacterium, a fungus, or a mammal. For example, in certain embodiments, the antigen is associated with an autoimmune disease, allergy, or asthma. In other embodiments, the antigen is associated with cancer, herpes, influenza, hepatitis B, hepatitis C, human papilloma virus (HPV), ebola, pneumococcus, Haemophilus influenza, meningococcus, dengue, tuberculosis, malaria, norovirus or human immunodeficiency virus (HIV). In certain embodiments, the antigen comprises a consensus sequence based on the amino acid sequence of two or more different organisms. In certain embodiments, the nucleic acid sequence encoding the antigen is optimized for effective translation in the organism in which the composition is delivered.

In certain embodiments, the antigen comprises a tumor-specific antigen or tumor-associated antigen, such that the antigen induces an adaptive immune response against the tumor. In certain embodiments, the antigen comprises a fragment of a tumor-specific antigen or tumor-associated antigen, such that the antigen induces an adaptive immune response against the tumor. In certain embodiment, the tumor-specific antigen or tumor-associated antigen is a mutation variant of a host protein.

### Viral Antigens

In certain embodiments, the antigen comprises a viral antigen, or fragment thereof, or variant thereof. In certain embodiments, the viral antigen is from a virus from one of the following families: Adenoviridae, Arenaviridae, Bunyaviridae, Caliciviridae, Coronaviridae, Filoviridae, Hepadnaviridae, Herpesviridae, Orthomyxoviridae, Papovaviridae, Paramyxoviridae, Parvoviridae, Picornaviridae, Poxviridae, Reoviridae, Retroviridae, Rhabdoviridae, or Togaviridae. In certain embodiments, the viral antigen is from papilloma viruses, for example, human papillomoa virus (HPV), human immunodeficiency virus (HIV), polio virus, hepatitis B virus, hepatitis C virus, smallpox virus (Variola major and minor), vaccinia virus, influenza virus, rhinoviruses, dengue fever virus, equine encephalitis viruses, rubella virus, yellow fever virus, Norwalk virus, hepatitis A virus, human T-cell leukemia virus (HTLV-I), hairy cell leukemia virus (HTLV-II), California encephalitis virus, Hanta virus (hemorrhagic fever), rabies virus, Ebola fever virus, Marburg virus, measles virus, mumps virus, respiratory syncytial virus (RSV), herpes simplex 1 (oral herpes), herpes simplex 2 (genital herpes), herpes zoster (varicella-zoster, a.k.a., chickenpox), cytomegalovirus (CMV), for example human CMV, Epstein-Barr virus (EBV), flavivirus, foot and mouth disease virus, chikungunya virus, lassa virus, arenavirus, severe acute respiratory syndrome (SARS) virus, severe acute respiratory syndrome coronavirus 2 (SARS-CoV-2) or a cancer causing virus.

### Parasite Antigens

In certain embodiments, the antigen comprises a parasite antigen or fragment or variant thereof. In certain embodiments, the parasite is a protozoa, helminth, or ectoparasite.

In certain embodiments, the helminth (i.e., worm) is a flatworm (e.g., flukes and tapeworms), a thomy-headed worm, or a round worm (e.g., pinworms). In certain embodiments, the ectoparasite is lice, fleas, ticks, and mites.

In certain embodiments, the parasite is any parasite causing the following diseases: Acanthamoeba keratitis, Amoebiasis, Ascariasis, Babesiosis, Balantidiasis, Baylisascariasis, Chagas disease, Clonorchiasis, Cochliomyia, Cryptosporidiosis, Diphyllobothriasis, Dracunculiasis, Echinococcosis, Elephantiasis, Enterobiasis, Fascioliasis, Fasciolopsiasis, Filariasis, Giardiasis, Gnathostomiasis, Hymenolepiasis, Isosporiasis, Katayama fever, Leishmaniasis, Lyme disease, Malaria, Metagonimiasis, Myiasis, Onchocerciasis, Pediculosis, Scabies, Schistosomiasis, Sleeping sickness, Strongyloidiasis, Taeniasis, Toxocariasis, Toxoplasmosis, Trichinosis, and Trichuriasis.

In certain embodiments, the parasite is Acanthamoeba, Anisakis, Ascaris lumbricoides, Botfly, Balantidium coli, Bedbug, Cestoda (tapeworm), Chiggers, Cochliomyia hominivorax, Entamoeba histolytica, Fasciola hepatica, Giardia lamblia, Hookworm, Leishmania, Linguatula serrata, Liver fluke, Loa loa, Paragonimus—lung fluke, Pinworm, Plasmodium falciparum, Schistosoma, Strongyloides stercoralis, Mite, Tapeworm, Toxoplasma gondii, Trypanosoma, Whipworm, or Wuchereria bancrofti.

### Bacterial Antigens

In certain embodiments, the antigen comprises a bacterial antigen or fragment or variant thereof. In certain embodiments, the bacterium is from any one of the following phyla: Acidobacteria, Actinobacteria, Aquificae, Bacteroidetes, Caldiserica, Chlamydiae, Chlorobi, Chloroflexi, Chrysiogenetes, Cyanobacteria, Deferribacteres, Deinococcus-Thermus, Dictyoglomi, Elusimicrobia, Fibrobacteres, Firmicutes, Fusobacteria, Gemmatimonadetes, Lentisphaerae, Nitrospira, Planctomycetes, Proteobacteria, Spirochaetes, Synergistetes, Tenericutes, Thermodesulfobacteria, Thermotogae, and Verrucomicrobia.

In certain embodiments, the bacterium is a gram positive bacterium or a gram negative bacterium. In certain embodiments, the bacterium is an aerobic bacterium or an anaerobic bacterium. In certain embodiments, the bacterium is an autotrophic bacterium or a heterotrophic bacterium. In certain embodiments, the bacterium is a mesophile, a neutrophile, an extremophile, an acidophile, an alkaliphile, a thermophile, psychrophile, halophile, or an osmophile.

In certain embodiments, the bacterium is an anthrax bacterium, an antibiotic resistant bacterium, a disease causing bacterium, a food poisoning bacterium, an infectious bacterium, Salmonella bacterium, Staphylococcus bacterium, Streptococcus bacterium, or tetanus bacterium. In certain embodiments, bacterium is a mycobacteria, Clostridium tetani, Yersinia pestis, Bacillus anthracis, methicillin-resistant Staphylococcus aureus (MRSA), or Clostridium difficile.

### Fungal Antigens

In certain embodiments, the antigen comprises a fungal antigen or fragment or variant thereof. In certain embodiments, the fungus is Aspergillus species, Blastomyces dermatitidis, Candida yeasts (e.g., Candida albicans), Coccidioides, Cryptococcus neoformans, Cryptococcus gattii, dermatophyte, Fusarium species, Histoplasma capsulatum, Mucoromycotina, Pneumocystis jirovecii, Sporothrix schenckii, Exserohilum, or Cladosporium.

### Tumor Antigens

In certain embodiments, the antigen comprises a tumor antigen, including for example a tumor-associated antigen or a tumor-specific antigen. In the context of the present disclosure, “tumor antigen” or “hyperporoliferative disorder antigen” or “antigen associated with a hyperproliferative disorder” refer to antigens that are common to specific hyperproliferative disorders. In certain aspects, the hyperproliferative disorder antigens of the present disclosure are derived from cancers including, but not limited to, primary or metastatic melanoma, mesothelioma, thymoma, lymphoma, sarcoma, lung cancer, liver cancer, non-Hodgkin's lymphoma, Hodgkins lymphoma, leukemias, uterine cancer, cervical cancer, bladder cancer, kidney cancer and adenocarcinomas such as breast cancer, prostate cancer, ovarian cancer, pancreatic cancer, and the like.

Tumor antigens are proteins that are produced by tumor cells that elicit an immune response, particularly T-cell mediated immune responses. In certain embodiments, the tumor antigen of the present disclosure comprises one or more antigenic cancer epitopes immunogenically recognized by tumor infiltrating lymphocytes (TIL) derived from a cancer tumor of a mammal. The selection of the antigen will depend on the particular type of cancer to be treated or prevented by way of the composition of the disclosure.

Tumor antigens are well known in the art and include, for example, a glioma-associated antigen, carcinoembryonic antigen (CEA), j-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-la, p53, prostein, PSMA, Her2/neu, survivin and telomerase, prostate-carcinoma tumor antigen-I (PCTA-1), MAGE, ELF2M, neutrophil elastase, ephrinB2, CD22, insulin growth factor (IGF)-I, IGF-II, IGF-I receptor and mesothelin.

In certain embodiments, the tumor antigen comprises one or more antigenic cancer epitopes associated with a malignant tumor. Malignant tumors express a number of proteins that can serve as target antigens for an immune attack. These molecules include but are not limited to tissue-specific antigens such as MART-1, tyrosinase and GP 100 in melanoma and prostatic acid phosphatase (PAP) and prostate-specific antigen (PSA) in prostate cancer. Other target molecules belong to the group of transformation-related molecules such as the oncogene HER-2/Neu/ErbB-2. Yet another group of target antigens are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. B-cell differentiation antigens such as CD19, CD20 and CD37 are other candidates for target antigens in B-cell lymphoma. Some of these antigens (CEA, HER-2, CD19, CD20, idiotype) have been used as targets for passive immunotherapy with monoclonal antibodies with limited success.

The type of tumor antigen referred to in the disclosure may also be a tumor-specific antigen (TSA) or a tumor-associated antigen (TAA). A TSA is unique to tumor cells and does not occur on other cells in the body. A TAA associated antigen is not unique to a tumor cell and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development when the immune system is immature and unable to respond or they may be antigens that are normally present at extremely low levels on normal cells but which are expressed at much higher levels on tumor cells.

Non-limiting examples of TSA or TAA antigens include the following: Differentiation antigens such as MART-1/MelanA (MART-I), gp100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, and TPS.

In a preferred embodiment, the antigen includes but is not limited to CD19, CD20, CD22, ROR1, Mesothelin, CD33/IL3Ra, c-Met, PSMA, Glycolipid F77, EGFRvIII, GD-2, MY-ESO-1 TCR, MAGE A3 TCR, and the like.

### Adjuvant

In certain embodiments, the composition comprises an adjuvant. In certain embodiments, the composition comprises a nucleic acid molecule encoding an adjuvant. In certain embodiments, the adjuvant-encoding nucleic acid molecule is IVT RNA. In certain embodiments, the adjuvant-encoding nucleic acid molecule is nucleoside-modified mRNA.

Exemplary adjuvants include, but is not limited to, alpha-interferon, gamma-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHC, CD80, CD86 including IL-15 having the signal sequence deleted and optionally including the signal peptide from IgE. Other genes which may be useful adjuvants include those encoding: MCP-I, MIP-Ia, MIP-Ip, IL-8, RANTES, L-selectin, P-selectin, E-selectin, CD34, GlyCAM-1, MadCAM-1, LFA-I, VLA-I, Mac-1, p150.95, PECAM, ICAM-I, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Fit, Apo-1, p55, WSL-I, DR3, TRAMP, Apo-3, AIR, LARD, NGRF, DR4, DR5, KILLER, TRAIL-R₂, TRICK2, DR6, Caspase ICE, Fos, c-jun, Sp-I, Ap-I, Ap-2, p38, p65Rel, MyD88, IRAK, TRAF6, IkB, Inactive NIK, SAP K, SAP-I, JNK, interferon response genes, NFkB, Bax, TRAIL, TRAILrec, TRAILrecDRC5, TRAIL-R₃, TRAIL-R₄, RANK, RANK LIGAND, Ox40, Ox40 LIGAND, NKG2D, MICA, MICB, NKG2A, NKG2B, NKG2C, NKG2E, NKG2F, TAP 1, TAP2, anti-CTLA4-sc, anti-LAG3-Ig, anti-TIM3-Ig and functional fragments thereof.

### Pharmaceutical Compositions

In another aspect, the present disclosure provides a composition comprising at least one LNP of the present disclosure and a pharmaceutically acceptable carrier. In certain embodiments, the composition further comprises an adjuvant. In certain embodiments, the composition is a vaccine.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the disclosure is contemplated include, but are not limited to, humans and other primates, mammals including commercially relevant mammals such as non-human primates, cattle, pigs, horses, sheep, cats, and dogs.

Pharmaceutical compositions that are useful in the methods of the disclosure may be prepared, packaged, or sold in formulations suitable for ophthalmic, oral, rectal, vaginal, parenteral, topical, pulmonary, intranasal, buccal, intravenous, intracerebroventricular, intradermal, intramuscular, or another route of administration. Other contemplated formulations include projected nanoparticles, liposomal preparations, resealed erythrocytes containing the active ingredient, and immunogenic-based formulations.

A pharmaceutical composition of the disclosure may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the disclosure will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the disclosure may further comprise one or more additional pharmaceutically active agents.

Controlled- or sustained-release formulations of a pharmaceutical composition of the disclosure may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intradermal, intrastemal injection, intratumoral, intravenous, intracerebroventricular and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In certain embodiments of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the disclosure may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In certain embodiments of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations that are useful include those that comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the disclosure are known in the art and described, for example in Remington's Pharmaceutical Sciences (1985, Genaro, ed., Mack Publishing Co., Easton, PA), which is incorporated herein by reference.

### Methods

The present disclosure provides methods of delivering an agent to a cell, tissue or organ of a target fetal subject in utero. In some embodiments, the agent is a diagnostic agent to detect at least one marker associated with a disease or disorder. In some embodiments, the agent is a therapeutic agent for the treatment or prevention of a disease or disorder. Therefore, in some embodiments, the disclosure provides methods for diagnosing, treating or preventing a disease or disorder comprising administering an effective amount of a composition comprising one or more diagnostic or therapeutic agents, one or more adjuvants, or a combination thereof.

In some embodiments, the method provides for delivery of compositions for gene editing or genetic manipulation to a target fetal subject to treat or prevent a genetic or congenital disease or disorder. Exemplary genetic or congenital diseases or disorders include, but are not limited to, β-thalassemia, cystic fibrosis, glycogen storage disorders, cleft lip and cleft palate, cerebral palsy, Fragile X syndrome, Down syndrome, spina bifida, congenital heart defects, genetic lung diseases such as surfactant deficiencies, genetic skin diseases, amniotic membrane rupture and amniotic membrane diseases.

In certain embodiments, the composition is administered to a maternal subject for in utero treatment of a target fetal subject. In certain embodiments, the composition is administered to a maternal subject following genetic testing determining that a target fetal subject has or is at increased risk of developing a congenital defect. In certain embodiments, the composition is administered to a maternal subject who has been diagnosed as having a disease or disorder that adversely impacts fetal development.

In some embodiments, the method provides immunity in the target maternal or fetal subject to an infection, disease, or disorder associated with an antigen. The present disclosure thus provides a method of treating or preventing the infection, disease, or disorder associated with the antigen. For example, the method may be used to treat or prevent a viral infection, bacterial infection, fungal infection, parasitic infection, or cancer, depending upon the type of antigen of the administered composition. Exemplary antigens and associated infections, diseases, and tumors are described elsewhere herein.

In certain embodiments, the composition is administered to a target maternal or fetal subject having an infection, disease, or cancer associated with the antigen. In certain embodiments, the composition is administered to a subject at risk for developing the infection, disease, or cancer associated with the antigen. For example, the composition may be administered to a subject who is at risk for being in contact with a virus, bacteria, fungus, parasite, or the like. For example, In certain embodiments, the composition is administered to a maternal subject at risk for being in contact with ZIKA virus to treat or prevent development of a ZIKA-virus related congenital disease or disorder in a target fetal subject.

In certain embodiments, the method comprises administering a composition comprising one or more nucleoside-modified nucleic acid molecules encoding one or more antigens and one or more adjuvant. In certain embodiments, the method comprises administering a composition comprising a first nucleoside-modified nucleic acid molecule encoding one or more antigens and a second nucleoside-modified nucleic acid molecule encoding one or more adjuvants. In certain embodiments, the method comprises administering a first composition comprising one or more nucleoside-modified nucleic acid molecules encoding one or more antigens and administering a second composition comprising one or more nucleoside-modified nucleic acid molecules encoding one or more adjuvants.

In certain embodiments, the method comprises administering to subject a plurality of nucleoside-modified nucleic acid molecules encoding a plurality of antigens, adjuvants, or a combination thereof.

In certain embodiments, the method of the disclosure allows for sustained expression of the antigen or adjuvant, described herein, for at least several days following administration. However, the method, in certain embodiments, also provides for transient expression, as in certain embodiments, the nucleic acid is not integrated into the subject genome.

In certain embodiments, the method comprises administering nucleoside-modified RNA which provides stable expression of the antigen or adjuvant described herein. In some embodiments, administration of nucleoside-modified RNA results in little to no innate immune response, while inducing an effective adaptive immune response.

Administration of the compositions of the disclosure in a method of treatment can be achieved in a number of different ways, using methods known in the art. In certain embodiments, the method of the disclosure comprises systemic administration of the subject, including for example enteral or parenteral administration. In certain embodiments, the method comprises intradermal delivery of the composition. In other embodiments, the method comprises intravenous delivery of the composition. In some embodiments, the method comprises intramuscular delivery of the composition. In certain embodiments, the method comprises subcutaneous delivery of the composition. In certain embodiments, the method comprises inhalation of the composition. In certain embodiments, the method comprises intranasal delivery of the composition.

It will be appreciated that the composition of the disclosure may be administered to a subject either alone, or in conjunction with another agent.

The therapeutic and prophylactic methods of the disclosure thus encompass the use of pharmaceutical compositions encoding an antigen, adjuvant, or a combination thereof, described herein to practice the methods of the disclosure. The pharmaceutical compositions useful for practicing the disclosure may be administered to deliver a dose of from ng/kg/day and 100 mg/kg/day. In certain embodiments, the disclosure envisions administration of a dose which results in a concentration of the compound of the present disclosure from 10 nM and 10 M in a mammal.

Typically, dosages which may be administered in a method of the disclosure to a mammal, preferably a human, range in amount from 0.01 μg to about 50 mg per kilogram of body weight of the mammal, while the precise dosage administered will vary depending upon any number of factors, including but not limited to, the type of mammal and type of disease state being treated, the age of the mammal and the route of administration. Preferably, the dosage of the compound will vary from about 0.1 pg to about 10 mg per kilogram of body weight of the mammal. More preferably, the dosage will vary from about 1 pg to about 1 mg per kilogram of body weight of the mammal.

The composition may be administered to a mammal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the mammal, etc.

In certain embodiments, administration of an immunogenic composition or vaccine of the present disclosure may be performed by single administration or boosted by multiple administrations.

In certain embodiments, the disclosure includes a method comprising administering one or more compositions encoding one or more antigens or adjuvants described herein. In certain embodiments, the method has an additive effect, wherein the overall effect of the administering the combination is approximately equal to the sum of the effects of administering each antigen or adjuvant. In other embodiments, the method has a synergistic effect, wherein the overall effect of administering the combination is greater than the sum of the effects of administering each antigen or adjuvant.

In another aspect, the present disclosure provides a method of delivering a nucleic acid molecule, therapeutic agent, or a combination thereof to a fetal subject in utero, the method comprising administering a therapeutically effectively amount of at least one LNP of the present disclosure or at least one composition of the present disclosure to a maternal subject comprising the fetal subject.

In certain embodiments, the nucleic acid molecule is a therapeutic agent.

In certain embodiments, the nucleic acid molecule is a DNA molecule or an RNA molecule.

In certain embodiments, the nucleic acid molecule is selected from the group consisting of cDNA, mRNA, miRNA, siRNA, antagomir, antisense molecule, peptide, therapeutic peptide, targeted nucleic acid, and any combination thereof.

In certain embodiments, the nucleic acid molecule encodes one or more components for gene editing.

In certain embodiments, the mRNA encodes one or more antigens.

In certain embodiments, the LNP of the present disclosure or the composition of the present disclosure further comprises an adjuvant.

In certain embodiments, the nucleic acid molecule, therapeutic agent, or combination thereof is encapsulated within the LNP.

In certain embodiments, the LNP or the composition thereof is administered by in utero delivery.

In certain embodiments, the method treats or prevents at least one selected from the group consisting of a viral infection, a bacterial infection, a fungal infection, a parasitic infection, influenza infection, cancer, arthritis, heart disease, cardiovascular disease, neurological disorder or disease, genetic disease, autoimmune disease, and fetal disease, genetic disease affecting fetal development, and any combination thereof.

In another aspect, the present disclosure provides a method of delivering a nucleic acid molecule to a fetal cell, the method comprising in utero administration of a therapeutically effectively amount of at least one LNP of the present disclosure or at least one composition of the present disclosure comprising to a maternal subject comprising the fetal cell.

In certain embodiments, the method is a gene delivery method.

In another aspect, the present disclosure provides a method of preventing or treating a disease or disorder in a target fetal subject, the method comprising in utero administration of a therapeutically effectively amount of at least one LNP of the present disclosure or at least one composition of the present disclosure to a maternal subject comprising the fetal subject.

In certain embodiments, the LNP or the composition thereof delivers the nucleic acid molecule, therapeutic agent, or combination thereof to a fetal cell.

In certain embodiments, the disease or disorder is selected from the group consisting of β-thalassemia, cystic fibrosis, glycogen storage disorders, cleft lip and cleft palate, cerebral palsy, Fragile X syndrome, Down syndrome, spina bifida, congenital heart defects, genetic lung diseases, genetic skin diseases, amniotic membrane rupture, and amniotic membrane diseases.

In another aspect, the present disclosure provides a method of identifying a LNP as having increased stability in a test fluid. In certain embodiments, the method comprises contacting at least one LNP with a first concentration of a test fluid. In certain embodiments, the method comprises determining the size and polydispersity index (PDI) of the LNP using dynamic light scattering (DLS). In certain embodiments, the method comprises contacting the at least one LNP to be tested with at least one additional concentration of the fluid. In certain embodiments, the method comprises determining the size and polydispersity index (PDI) of the LNP using dynamic light scattering (DLS) in the presence of the at least one additional concentration of the fluid. In certain embodiments, the method comprises comparing the size and polydispersity index (PDI) of the LNP at the at least two fluid concentrations. In certain embodiments, the method comprises identifying the test LNP as having stability in the test fluid based on the changes in size and polydispersity index (PDI) of the LNP.

In certain embodiments, the test fluid mimics a biological fluid.

In certain embodiments, the biological fluid is amniotic fluid or an amniotic fluid mimic.

In certain embodiments, the method is a screening method for detecting LNPs having increased stability.

In certain embodiments, the test LNP is identified as having stability in the test fluid based a lower level of change in at least one selected from the group consisting of size and PDI of the LNP as compared to a control LNP.

### Small Molecule Therapeutic Agents

In various embodiments, the agent is a therapeutic agent. In various embodiments, the therapeutic agent is a small molecule. When the therapeutic agent is a small molecule, a small molecule may be obtained using standard methods known to the skilled artisan. Such methods include chemical organic synthesis or biological means. Biological means include purification from a biological source, recombinant synthesis and in vitro translation systems, using methods well known in the art. In certain embodiments, a small molecule therapeutic agents comprises an organic molecule, inorganic molecule, biomolecule, synthetic molecule, and the like.

Combinatorial libraries of molecularly diverse chemical compounds potentially useful in treating a variety of diseases and conditions are well known in the art, as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development. In some embodiments of the disclosure, the therapeutic agent is synthesized and/or identified using combinatorial techniques.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores. In some embodiments of the disclosure, the therapeutic agent is synthesized via small library synthesis.

The small molecule and small molecule compounds described herein may be present as salts even if salts are not depicted, and it is understood that the disclosure embraces all salts and solvates of the therapeutic agents depicted here, as well as the non-salt and non-solvate form of the therapeutic agents, as is well understood by the skilled artisan. In some embodiments, the salts of the therapeutic agents of the disclosure are pharmaceutically acceptable salts.

Where tautomeric forms may be present for any of the therapeutic agents described herein, each and every tautomeric form is intended to be included in the present disclosure, even though only one or some of the tautomeric forms may be explicitly depicted. For example, when a 2-hydroxypyridyl moiety is depicted, the corresponding 2-pyridone tautomer is also intended.

The disclosure also includes any or all of the stereochemical forms, including any enantiomeric or diastereomeric forms of the therapeutic agents described. The recitation of the structure or name herein is intended to embrace all possible stereoisomers of therapeutic agents depicted. All forms of the therapeutic agents are also embraced by the disclosure, such as crystalline or non-crystalline forms of the therapeutic agent. Compositions comprising a therapeutic agents of the disclosure are also intended, such as a composition of substantially pure therapeutic agent, including a specific stereochemical form thereof, or a composition comprising mixtures of therapeutic agents of the disclosure in any ratio, including two or more stereochemical forms, such as in a racemic or non-racemic mixture.

The disclosure also includes any or all active analog or derivative, such as a prodrug, of any therapeutic agent described herein. In certain embodiments, the therapeutic agent is a prodrug. In certain embodiments, the small molecules described herein are candidates for derivatization. As such, in certain instances, the analogs of the small molecules described herein that have modulated potency, selectivity, and solubility are included herein and provide useful leads for drug discovery and drug development. Thus, in certain instances, during optimization new analogs are designed considering issues of drug delivery, metabolism, novelty, and safety.

In some instances, small molecule therapeutic agents described herein are derivatives or analogs of known therapeutic agents, as is well known in the art of combinatorial and medicinal chemistry. The analogs or derivatives can be prepared by adding and/or substituting functional groups at various locations. As such, the small molecules described herein can be converted into derivatives/analogs using well known chemical synthesis procedures. For example, all of the hydrogen atoms or substituents can be selectively modified to generate new analogs. Also, the linking atoms or groups can be modified into longer or shorter linkers with carbon backbones or hetero atoms. Also, the ring groups can be changed so as to have a different number of atoms in the ring and/or to include hetero atoms. Moreover, aromatics can be converted to cyclic rings, and vice versa. For example, the rings may be from 5-7 atoms, and may be carbocyclic or heterocyclic.

As used herein, the term “analog,” “analogue,” or “derivative” is meant to refer to a chemical compound or molecule made from a parent compound or molecule by one or more chemical reactions. As such, an analog can be a structure having a structure similar to that of the small molecule therapeutic agents described herein or can be based on a scaffold of a small molecule therapeutic agents described herein, but differing from it in respect to certain components or structural makeup, which may have a similar or opposite action metabolically. An analog or derivative of any of a small molecule inhibitor in accordance with the present disclosure can be used to treat a disease or disorder.

In certain embodiments, the small molecule therapeutic agents described herein can independently be derivatized, or analogs prepared therefrom, by modifying hydrogen groups independently from each other into other substituents. That is, each atom on each molecule can be independently modified with respect to the other atoms on the same molecule. Any traditional modification for producing a derivative/analog can be used. For example, the atoms and substituents can be independently comprised of hydrogen, an alkyl, aliphatic, straight chain aliphatic, aliphatic having a chain hetero atom, branched aliphatic, substituted aliphatic, cyclic aliphatic, heterocyclic aliphatic having one or more hetero atoms, aromatic, heteroaromatic, polyaromatic, polyamino acids, peptides, polypeptides, combinations thereof, halogens, halo-substituted aliphatics, and the like. Additionally, any ring group on a compound can be derivatized to increase and/or decrease ring size as well as change the backbone atoms to carbon atoms or hetero atoms.

### Nucleic Acid Therapeutic Agents

In other related aspects, the therapeutic agent is an isolated nucleic acid. In certain embodiments, the isolated nucleic acid molecule is one of a DNA molecule or an RNA molecule. In certain embodiments, the isolated nucleic acid molecule is a cDNA, mRNA, siRNA, shRNA or miRNA molecule. In certain embodiments, the isolated nucleic acid molecule encodes a therapeutic peptide such a thrombomodulin, endothelial protein C receptor (EPCR), anti-thrombotic proteins including plasminogen activators and their mutants, antioxidant proteins including catalase, superoxide dismutase (SOD) and iron-sequestering proteins. In some embodiments, the therapeutic agent is an siRNA, miRNA, shRNA, or an antisense molecule, which inhibits a targeted nucleic acid including those encoding proteins that are involved in aggravation of the pathological processes.

In certain embodiments, the nucleic acid comprises a promoter/regulatory sequence such that the nucleic acid is capable of directing expression of the nucleic acid. Thus, the disclosure encompasses expression vectors and methods for the introduction of exogenous nucleic acid into cells with concomitant expression of the exogenous nucleic acid in the cells such as those described, for example, in Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York) and as described elsewhere herein.

In certain embodiments, siRNA is used to decrease the level of a targeted protein. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391(19):306-311; Timmons et al., 1998, Nature 395:854; Montgomery et al., 1998, TIG 14 (7):255-258; David R. Engelke, Ed., RNA Interference (RNAi) Nuts & Bolts of RNAi Technology, DNA Press, Eagleville, P A (2003); and Gregory J. Hannon, Ed., RNAi A Guide to Gene Silencing, Cold Spring Harbor Laboratory Press, Cold Spring Harbor, NY (2003). Soutschek et al. (2004, Nature 432:173-178) describe a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216. Therefore, the present disclosure also includes methods of decreasing levels of PTPN22 using RNAi technology.

In one aspect, the disclosure includes a vector comprising an siRNA or an antisense polynucleotide. Preferably, the siRNA or antisense polynucleotide is capable of inhibiting the expression of a target polypeptide. The incorporation of a desired polynucleotide into a vector and the choice of vectors are well-known in the art as described in, for example, Sambrook et al. (2012), and in Ausubel et al. (1997), and elsewhere herein.

In certain embodiments, the expression vectors described herein encode a short hairpin RNA (shRNA) therapeutic agents. shRNA molecules are well known in the art and are directed against the mRNA of a target, thereby decreasing the expression of the target. In certain embodiments, the encoded shRNA is expressed by a cell, and is then processed into siRNA. For example, in certain instances, the cell possesses native enzymes (e.g., dicer) that cleave the shRNA to form siRNA.

In order to assess the expression of the siRNA, shRNA, or antisense polynucleotide, the expression vector to be introduced into a cell can also contain either a selectable marker gene or a reporter gene or both to facilitate identification of expressing cells from the population of cells sought to be transfected or infected using a the delivery vehicle of the disclosure. In other embodiments, the selectable marker may be carried on a separate piece of DNA and also be contained within the delivery vehicle. Both selectable markers and reporter genes may be flanked with appropriate regulatory sequences to enable expression in the host cells. Useful selectable markers are known in the art and include, for example, antibiotic-resistance genes, such as neomycin resistance and the like.

Therefore, in one aspect, the delivery vehicle may contain a vector, comprising the nucleotide sequence or the construct to be delivered. The choice of the vector will depend on the host cell in which it is to be subsequently introduced. In a particular embodiment, the vector of the disclosure is an expression vector. Suitable host cells include a wide variety of prokaryotic and eukaryotic host cells. In specific embodiments, the expression vector is selected from the group consisting of a viral vector, a bacterial vector and a mammalian cell vector. Prokaryote- and/or eukaryote-vector based systems can be employed for use with the present disclosure to produce polynucleotides, or their cognate polypeptides. Many such systems are commercially and widely available.

By way of illustration, the vector in which the nucleic acid sequence is introduced can be a plasmid, which is or is not integrated in the genome of a host cell when it is introduced in the cell. Illustrative, non-limiting examples of vectors in which the nucleotide sequence of the disclosure or the gene construct of the disclosure can be inserted include a tet-on inducible vector for expression in eukaryote cells.

The vector may be obtained by conventional methods known by persons skilled in the art (Sambrook et al., 2012). In a particular embodiment, the vector is a vector useful for transforming animal cells.

In certain embodiments, the recombinant expression vectors may also contain nucleic acid molecules, which encode a peptide or peptidomimetic.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2012). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

The recombinant expression vectors may also contain a selectable marker gene, which facilitates the selection of host cells. Suitable selectable marker genes are genes encoding proteins such as G418 and hygromycin, which confer resistance to certain drugs, β-galactosidase, chloramphenicol acetyltransferase, firefly luciferase, or an immunoglobulin or portion thereof such as the Fc portion of an immunoglobulin preferably IgG. The selectable markers may be introduced on a separate vector from the nucleic acid of interest.

Following the generation of the siRNA polynucleotide, a skilled artisan will understand that the siRNA polynucleotide will have certain characteristics that can be modified to improve the siRNA as a therapeutic compound. Therefore, the siRNA polynucleotide may be further designed to resist degradation by modifying it to include phosphorothioate, or other linkages, methylphosphonate, sulfone, sulfate, ketyl, phosphorodithioate, phosphoramidate, phosphate esters, and the like (see, e.g., Agrawal et al., 1987, Tetrahedron Lett. 28:3539-3542; Stec et al., 1985 Tetrahedron Lett. 26:2191-2194; Moody et al., 1989 Nucleic Acids Res. 12:4769-4782; Eckstein, 1989 Trends Biol. Sci. 14:97-100; Stein, In: Oligodeoxynucleotides. Antisense Inhibitors of Gene Expression, Cohen, ed., Macmillan Press, London, pp. 97-117 (1989)).

Any polynucleotide may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queuosine, and wybutosine and the like, as well as acetyl-methyl-, thio- and other modified forms of adenine, cytidine, guanine, thymine, and uridine.

In certain embodiments of the disclosure, an antisense nucleic acid sequence, which is expressed by a plasmid vector is used as a therapeutic agent to inhibit the expression of a target protein. The antisense expressing vector is used to transfect a mammalian cell or the mammal itself, thereby causing reduced endogenous expression of the target protein.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the disclosure may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the disclosure include oligonucleotide derivatives known in the art which have improved biological activity compared to unmodified oligonucleotides (see U.S. Pat. No. 5,023,243).

In certain embodiments of the disclosure, a ribozyme is used as a therapeutic agent to inhibit expression of a target protein. Ribozymes useful for inhibiting the expression of a target molecule may be designed by incorporating target sequences into the basic ribozyme structure, which are complementary, for example, to the mRNA sequence encoding the target molecule. Ribozymes targeting the target molecule, may be synthesized using commercially available reagents (Applied Biosystems, Inc., Foster City, CA) or they may be genetically expressed from DNA encoding them.

In certain embodiments, the therapeutic agent may comprise one or more components of a CRISPR-Cas system, where a guide RNA (gRNA) targeted to a gene encoding a target molecule, and a CRISPR-associated (Cas) peptide form a complex to induce mutations within the targeted gene. In certain embodiments, the therapeutic agent comprises a gRNA or a nucleic acid molecule encoding a gRNA. In certain embodiments, the therapeutic agent comprises a Cas peptide or a nucleic acid molecule encoding a Cas peptide.

In certain embodiments, the agent comprises a miRNA or a mimic of a miRNA. In certain embodiments, the agent comprises a nucleic acid molecule that encodes a miRNA or mimic of a miRNA.

MiRNAs are small non-coding RNA molecules that are capable of causing post-transcriptional silencing of specific genes in cells by the inhibition of translation or through degradation of the targeted mRNA. A miRNA can be completely complementary or can have a region of noncomplementarity with a target nucleic acid, consequently resulting in a “bulge” at the region of non-complementarity. A miRNA can inhibit gene expression by repressing translation, such as when the miRNA is not completely complementary to the target nucleic acid, or by causing target RNA degradation, which is believed to occur only when the miRNA binds its target with perfect complementarity. The disclosure also can include double-stranded precursors of miRNA. A miRNA or pri-miRNA can be 18-100 nucleotides in length, or from 18-80 nucleotides in length. Mature miRNAs can have a length of 19-30 nucleotides, or 21-25 nucleotides, particularly 21, 22, 23, 24, or 25 nucleotides. MiRNA precursors typically have a length of about 70-100 nucleotides and have a hairpin conformation. miRNAs are generated in vivo from pre-miRNAs by the enzymes Dicer and Drosha, which specifically process long pre-miRNA into functional miRNA. The hairpin or mature microRNAs, or pri-microRNA agents featured in the disclosure can be synthesized in vivo by a cell-based system or in vitro by chemical synthesis.

In various embodiments, the agent comprises an oligonucleotide that comprises the nucleotide sequence of a disease-associated miRNA. In certain embodiments, the oligonucleotide comprises the nucleotide sequence of a disease-associated miRNA in a pre-microRNA, mature or hairpin form. In other embodiments, a combination of oligonucleotides comprising a sequence of one or more disease-associated miRNAs, any pre-miRNA, any fragment, or any combination thereof is envisioned.

MiRNAs can be synthesized to include a modification that imparts a desired characteristic. For example, the modification can improve stability, hybridization thermodynamics with a target nucleic acid, targeting to a particular tissue or cell-type, or cell permeability, e.g., by an endocytosis-dependent or -independent mechanism.

Modifications can also increase sequence specificity, and consequently decrease off-site targeting. Methods of synthesis and chemical modifications are described in greater detail below. If desired, miRNA molecules may be modified to stabilize the miRNAs against degradation, to enhance half-life, or to otherwise improve efficacy. Desirable modifications are described, for example, in U.S. Patent Publication Nos. 20070213292, 20060287260, 20060035254. 20060008822, and 2005028824, each of which is hereby incorporated by reference in its entirety. For increased nuclease resistance and/or binding affinity to the target, the single-stranded oligonucleotide agents featured in the disclosure can include 2′-O-methyl, 2′-fluorine, 2′-O-methoxyethyl, 2′-O-aminopropyl, 2′-amino, and/or phosphorothioate linkages. Inclusion of locked nucleic acids (LNA), ethylene nucleic acids (ENA), e.g., 2′-4′-ethylene-bridged nucleic acids, and certain nucleotide modifications can also increase binding affinity to the target. The inclusion of pyranose sugars in the oligonucleotide backbone can also decrease endonucleolytic cleavage. An oligonucleotide can be further modified by including a 3′ cationic group, or by inverting the nucleoside at the 3′-terminus with a 3-3′ linkage. In another alternative, the 3′-terminus can be blocked with an aminoalkyl group. Other 3′ conjugates can inhibit 3′-5′ exonucleolytic cleavage. While not being bound by theory, a 3′ may inhibit exonucleolytic cleavage by sterically blocking the exonuclease from binding to the 3′ end of the oligonucleotide. Even small alkyl chains, aryl groups, or heterocyclic conjugates or modified sugars (D-ribose, deoxyribose, glucose etc.) can block 3′-5′-exonucleases.

In certain embodiments, the miRNA includes a 2′-modified oligonucleotide containing oligodeoxynucleotide gaps with some or all internucleotide linkages modified to phosphorothioates for nuclease resistance. The presence of methylphosphonate modifications increases the affinity of the oligonucleotide for its target RNA and thus reduces the ICSQ. This modification also increases the nuclease resistance of the modified oligonucleotide. It is understood that the methods and reagents of the present disclosure may be used in conjunction with any technologies that may be developed to enhance the stability or efficacy of an inhibitory nucleic acid molecule.

miRNA molecules include nucleotide oligomers containing modified backbones or non-natural internucleoside linkages. Oligomers having modified backbones include those that retain a phosphorus atom in the backbone and those that do not have a phosphorus atom in the backbone. For the purposes of this disclosure, modified oligonucleotides that do not have a phosphorus atom in their internucleoside backbone are also considered to be nucleotide oligomers. Nucleotide oligomers that have modified oligonucleotide backbones include, for example, phosphorothioates, chiral phosphorothioates, phosphorodithioates, phosphotriesters, aminoalkyl-phosphotriesters, methyl and other alkyl phosphonates including 3′-alkylene phosphonates and chiral phosphonates, phosphinates, phosphoramidates, thionophosphoramidates, thionoalkylphosphonates, thionoalkylphosphotriest-ers, and boranophosphates. Various salts, mixed salts and free acid forms are also included.

A miRNA described herein, which may be in the mature or hairpin form, may be provided as a naked oligonucleotide. In some cases, it may be desirable to utilize a formulation that aids in the delivery of a miRNA or other nucleotide oligomer to cells (see, e.g., U.S. Pat. Nos. 5,656,611, 5,753,613, 5,785,992, 6,120,798, 6,221,959, 6,346,613, and 6,353,055, each of which is hereby incorporated by reference).

In some examples, the miRNA composition is at least partially crystalline, uniformly crystalline, and/or anhydrous (e.g., less than 80, 50, 30, 20, or 10% water). In another example, the miRNA composition is in an aqueous phase, e.g., in a solution that includes water. The aqueous phase or the crystalline compositions can be incorporated into a delivery vehicle, e.g., a liposome (particularly for the aqueous phase), or a particle (e.g., a microparticle as can be appropriate for a crystalline composition). Generally, the miRNA composition is formulated in a manner that is compatible with the intended method of administration. A miRNA composition can be formulated in combination with another agent, e.g., another therapeutic agent or an agent that stabilizes an oligonucleotide agent, e.g., a protein that complexes with the oligonucleotide agent. Still other agents include chelators, e.g., EDTA (e.g., to remove divalent cations such as Mg), salts, and RNAse inhibitors (e.g., a broad specificity RNAse inhibitor). In certain embodiments, the miRNA composition includes another miRNA, e.g., a second miRNA composition (e.g., a microRNA that is distinct from the first). Still other preparations can include at least three, five, ten, twenty, fifty, or a hundred or more different oligonucleotide species.

In certain embodiments, the composition comprises an oligonucleotide composition that mimics the activity of a miRNA. In certain embodiments, the composition comprises oligonucleotides having nucleobase identity to the nucleobase sequence of a miRNA, and are thus designed to mimic the activity of the miRNA. In certain embodiments, the oligonucleotide composition that mimics miRNA activity comprises a double-stranded RNA molecule which mimics the mature miRNA hairpins or processed miRNA duplexes.

In certain embodiments, the oligonucleotide shares identity with endogenous miRNA or miRNA precursor nucleobase sequences. An oligonucleotide selected for inclusion in a composition of the present disclosure may be one of a number of lengths. Such an oligonucleotide can be from 7 to 100 linked nucleosides in length. For example, an oligonucleotide sharing nucleobase identity with a miRNA may be from 7 to 30 linked nucleosides in length. An oligonucleotide sharing identity with a miRNA precursor may be up to 100 linked nucleosides in length. In certain embodiments, an oligonucleotide comprises 7 to 30 linked nucleosides. In certain embodiments, an oligonucleotide comprises 7, 8, 9, 10, 11, 12, 13, 14, 15, 16, 17, 18, 19, 20, 21, 22, 23, 24, 25, 26, 28, 29, or 30 linked nucleotides. In certain embodiments, an oligonucleotide comprises 19 to 23 linked nucleosides. In certain embodiments, an oligonucleotide is from 40 up to 50, 60, 70, 80, 90, or 100 linked nucleosides in length.

In certain embodiments, an oligonucleotide has a sequence that has a certain identity to a miRNA or a precursor thereof. Nucleobase sequences of mature miRNAs and their corresponding stem-loop sequences described herein are the sequences found in miRBase, an online searchable database of miRNA sequences and annotation. Entries in the miRBase Sequence database represent a predicted hairpin portion of a miRNA transcript (the stem-loop), with information on the location and sequence of the mature miRNA sequence. The miRNA stem-loop sequences in the database are not strictly precursor miRNAs (pre-miRNAs), and may in some instances include the pre-miRNA and some flanking sequence from the presumed primary transcript. The miRNA nucleobase sequences described herein encompass any version of the miRNA, including the sequences described in Release 10.0 of the miRBase sequence database and sequences described in any earlier Release of the miRBase sequence database. A sequence database release may result in the re-naming of certain miRNAs. A sequence database release may result in a variation of a mature miRNA sequence. The compositions of the present disclosure encompass oligomeric compound comprising oligonucleotides having a certain identity to any nucleobase sequence version of a miRNAs described herein.

In certain embodiments, an oligonucleotide has a nucleobase sequence at least 60%, 65%, 70%, 75%, 80%, 85%, 90%, 95%, 97%, 98% or 99% identical to the miRNA over a region of 7, 8, 9, 10, 11, 12, 13, 14, 15, 16, 17, 18, 19, 20, 21, 22, 23, 24, 25, 26, 27, 28, 29, or 30 nucleobases. Accordingly, in certain embodiments the nucleobase sequence of an oligonucleotide may have one or more non-identical nucleobases with respect to the miRNA.

In certain embodiments, the composition comprises a nucleic acid molecule encoding a miRNA, precursor, mimic, or fragment thereof. For example, the composition may comprise a viral vector, plasmid, cosmid, or other expression vector suitable for expressing the miRNA, precursor, mimic, or fragment thereof in a desired mammalian cell or tissue.

### Vaccine

In certain embodiments, the present disclosure provides an immunogenic composition for inducing an immune response in a subject. For example, In certain embodiments, the immunogenic composition is a vaccine. As used herein, an “immunogenic composition” may comprise an antigen (e.g., a peptide or polypeptide), a nucleic acid encoding an antigen, a cell expressing or presenting an antigen or cellular component, or a combination thereof. In particular embodiments the composition comprises or encodes all or part of any peptide antigen, or an immunogenically functional equivalent thereof. In other embodiments, the composition comprises a mixture of mRNA molecules that encodes one or more additional immunostimulatory agent. Immunostimulatory agents include, but are not limited to, an additional antigen, an immunomodulator, or an adjuvant. In the context of the present disclosure, the term “vaccine” refers to a substance that induces immunity upon inoculation into animals.

A vaccine of the present disclosure may vary in its composition of nucleic acid components. In a non-limiting example, a nucleic acid encoding an antigen might also be formulated with an adjuvant. Of course, it will be understood that various compositions described herein may further comprise additional components. A vaccine of the present disclosure, and its various components, may be prepared and/or administered by any method disclosed herein or as would be known to one of ordinary skill in the art, in light of the present disclosure.

In some embodiments, the therapeutic compounds or compositions of the disclosure may be administered prophylactically (i.e., to prevent disease or disorder) or therapeutically (i.e., to treat disease or disorder) to subjects suffering from or at risk of (or susceptible to) developing the disease or disorder. Such subjects may be identified using standard clinical methods. In the context of the present disclosure, prophylactic administration occurs prior to the manifestation of overt clinical symptoms of disease, such that a disease or disorder is prevented or alternatively delayed in its progression. In the context of the field of medicine, the term “prevent” encompasses any activity which reduces the burden of mortality or morbidity from disease. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications.

### Nucleic Acids

In certain embodiments, the disclosure includes a ionizable LNP molecule formulated for in utero stability comprising or encapsulating one or more nucleic acid molecule. In certain embodiments, the nucleic acid molecule is a nucleoside-modified mRNA molecule. In certain embodiments, the nucleoside-modified mRNA molecule encodes an antigen. In certain embodiments, the nucleoside-modified mRNA molecule encodes a plurality of antigens. In certain embodiments, the nucleoside-modified mRNA molecule encodes an antigen that induces an adaptive immune response against the antigen. In certain embodiments, the disclosure includes a nucleoside-modified mRNA molecule encoding an adjuvant.

The nucleotide sequences encoding an antigen or adjuvant, as described herein, can alternatively comprise sequence variations with respect to the original nucleotide sequences, for example, substitutions, insertions and/or deletions of one or more nucleotides, with the condition that the resulting polynucleotide encodes a polypeptide according to the disclosure.

Therefore, the scope of the present disclosure includes nucleotide sequences that are substantially homologous to the nucleotide sequences recited herein and encode an antigen or adjuvant of interest.

As used herein, a nucleotide sequence is “substantially homologous” to any of the nucleotide sequences described herein when its nucleotide sequence has a degree of identity with respect to the nucleotide sequence of at least 60%, advantageously of at least 70%, preferably of at least 85%, and more preferably of at least 95%. A nucleotide sequence that is substantially homologous to a nucleotide sequence encoding an antigen can typically be isolated from a producer organism of the antigen based on the information contained in the nucleotide sequence by means of introducing conservative or non-conservative substitutions, for example. Other examples of possible modifications include the insertion of one or more nucleotides in the sequence, the addition of one or more nucleotides in any of the ends of the sequence, or the deletion of one or more nucleotides in any end or inside the sequence. The degree of identity between two polynucleotides is determined using computer algorithms and methods that are widely known for the persons skilled in the art.

Further, the scope of the disclosure includes nucleotide sequences that encode amino acid sequences that are substantially homologous to the amino acid sequences recited herein and preserve the immunogenic function of the original amino acid sequence.

As used herein, an amino acid sequence is “substantially homologous” to any of the amino acid sequences described herein when its amino acid sequence has a degree of identity with respect to the amino acid sequence of at least 60%, advantageously of at least 70%, preferably of at least 85%, and more preferably of at least 95%. The identity between two amino acid sequences is preferably determined by using the BLASTN algorithm (BLAST Manual, Altschul, S., et al., NCBI NLM NIH Bethesda, Md. 20894, Altschul, S., et al., J. Mol. Biol. 215: 403-410 (1990)).

In certain embodiments, the disclosure relates to a construct, comprising a nucleotide sequence encoding an antigen. In certain embodiments, the construct comprises a plurality of nucleotide sequences encoding a plurality of antigens. For example, in certain embodiments, the construct encodes 1 or more, 2 or more, 5 or more, 10 or more, 15 or more, or 20 or more antigens. In certain embodiments, the disclosure relates to a construct, comprising a nucleotide sequence encoding an adjuvant. In certain embodiments, the construct comprises a first nucleotide sequence encoding an antigen and a second nucleotide sequence encoding an adjuvant.

In certain embodiments, the composition comprises a plurality of constructs, each construct encoding one or more antigens. In certain embodiments, the composition comprises 1 or more, 2 or more, 5 or more, 10 or more, 15 or more, or 20 or more constructs. In certain embodiments, the composition comprises a first construct, comprising a nucleotide sequence encoding an antigen; and a second construct, comprising a nucleotide sequence encoding an adjuvant.

In another particular embodiment, the construct is operatively bound to a translational control element. The construct can incorporate an operatively bound regulatory sequence for the expression of the nucleotide sequence of the disclosure, thus forming an expression cassette.

### Vectors

The nucleic acid sequences encapsulated in the ionizable LNP molecule formulated for in utero stability of the disclosure can be obtained using recombinant methods known in the art, such as, for example by screening libraries from cells expressing the gene, by deriving the gene from a vector known to include the same, or by isolating directly from cells and tissues containing the same, using standard techniques. Alternatively, the nucleic acid molecule of interest can be produced synthetically.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, sequencing vectors and vectors optimized for in vitro transcription.

In certain embodiments, the composition of the disclosure comprises in vitro transcribed (IVT) RNA encoding an antigen. In certain embodiments, the composition of the disclosure comprises IVT RNA encoding a plurality of antigens. In certain embodiments, the composition of the disclosure comprises IVT RNA encoding an adjuvant. In certain embodiments, the composition of the disclosure comprises IVT RNA encoding one or more antigens and one or more adjuvants.

### Nucleoside-Modified RNA

In certain embodiments, the composition comprises a nucleoside-modified RNA. In certain embodiments, the composition comprises a nucleoside-modified mRNA. Nucleoside-modified mRNA have particular advantages over non-modified mRNA, including for example, increased stability, low or absent innate immunogenicity, and enhanced translation. Nucleoside-modified mRNA useful in the present disclosure is further described in U.S. Pat. No. 8,278,036, which is incorporated by reference herein in its entirety.

In certain embodiments, nucleoside-modified mRNA does not activate any pathophysiologic pathways, translates very efficiently and almost immediately following delivery, and serve as templates for continuous protein production in vivo lasting for several days (Karikó et al., 2008, Mol Ther 16:1833-1840; Karikó et al., 2012, Mol Ther 20:948-953). The amount of mRNA required to exert a physiological effect is small and that makes it applicable for human therapy. For example, as described herein, nucleoside-modified mRNA encoding an antigen has demonstrated the ability to induce CD4+ and CD8+ T-cell and antigen-specific antibody production. For example, in certain instances, antigen encoded by nucleoside-modified mRNA induces greater production of antigen-specific antibody production as compared to antigen encoded by non-modified mRNA.

In certain instances, expressing a protein by delivering the encoding mRNA has many benefits over methods that use protein, plasmid DNA or viral vectors. During mRNA transfection, the coding sequence of the desired protein is the only substance delivered to cells, thus avoiding all the side effects associated with plasmid backbones, viral genes, and viral proteins. More importantly, unlike DNA- and viral-based vectors, the mRNA does not carry the risk of being incorporated into the genome and protein production starts immediately after mRNA delivery. For example, high levels of circulating proteins have been measured within 15 to 30 minutes of in vivo injection of the encoding mRNA. In certain embodiments, using mRNA rather than the protein also has many advantages. Half-lives of proteins in the circulation are often short, thus protein treatment would need frequent dosing, while mRNA provides a template for continuous protein production for several days. Purification of proteins is problematic and they can contain aggregates and other impurities that cause adverse effects (Kromminga and Schellekens, 2005, Ann NY Acad Sci 1050:257-265).

In certain embodiments, the nucleoside-modified RNA comprises the naturally occurring modified-nucleoside pseudouridine. In certain embodiments, inclusion of pseudouridine makes the mRNA more stable, non-immunogenic, and highly translatable (Karikó et al., 2008, Mol Ther 16:1833-1840; Anderson et al., 2010, Nucleic Acids Res 38:5884-5892; Anderson et al., 2011, Nucleic Acids Research 39:9329-9338; Karikó et al., 2011, Nucleic Acids Research 39:e142; Karikó et al., 2012, Mol Ther 20:948-953; Karik6 et al., 2005, Immunity 23:165-175).

It has been demonstrated that the presence of modified nucleosides, including pseudouridines in RNA suppress their innate immunogenicity (Karikó et al., 2005, Immunity 23:165-175). Further, protein-encoding, in vitro-transcribed RNA containing pseudouridine can be translated more efficiently than RNA containing no or other modified nucleosides (Karikó et al., 2008, Mol Ther 16:1833-1840). Subsequently, it is shown that the presence of pseudouridine improves the stability of RNA (Anderson et al., 2011, Nucleic Acids Research 39:9329-9338) and abates both activation of PKR and inhibition of translation (Anderson et al., 2010, Nucleic Acids Res 38:5884-5892). A preparative HPLC purification procedure has been established that was critical to obtain pseudouridine-containing RNA that has superior translational potential and no innate immunogenicity (Karik6 et al., 2011, Nucleic Acids Research 39:e142). Administering HPLC-purified, pseudourine-containing RNA coding for erythropoietin into mice and macaques resulted in a significant increase of serum EPO levels (Karik6 et al., 2012, Mol Ther 20:948-953), thus confirming that pseudouridine-containing mRNA is suitable for in vivo protein therapy.

The present disclosure encompasses RNA, oligoribonucleotide, and polyribonucleotide molecules comprising pseudouridine or a modified nucleoside. In certain embodiments, the composition comprises an isolated nucleic acid encoding an antigen, wherein the nucleic acid comprises a pseudouridine or a modified nucleoside. In certain embodiments, the composition comprises a vector, comprising an isolated nucleic acid encoding an antigen, adjuvant, or combination thereof, wherein the nucleic acid comprises a pseudouridine or a modified nucleoside.

In certain embodiments, the nucleoside-modified RNA of the disclosure is IVT RNA. For example, in certain embodiments, the nucleoside-modified RNA is synthesized by T7 phage RNA polymerase. In other embodiments, the nucleoside-modified mRNA is synthesized by SP6 phage RNA polymerase. In other embodiments, the nucleoside-modified RNA is synthesized by T3 phage RNA polymerase.

In certain embodiments, the modified nucleoside is m¹acp³Ψ (1-methyl-3-(3-amino-3-carboxypropyl) pseudouridine. In other embodiments, the modified nucleoside is m¹Ψ (1-methylpseudouridine). In other embodiments, the modified nucleoside is Ψm (2′-O-methylpseudouridine. In other embodiments, the modified nucleoside is m⁵D (5-methyldihydrouridine). In other embodiments, the modified nucleoside is m³Ψ (3-methylpseudouridine). In other embodiments, the modified nucleoside is a pseudouridine moiety that is not further modified. In other embodiments, the modified nucleoside is a monophosphate, diphosphate, or triphosphate of any of the above pseudouridines. In other embodiments, the modified nucleoside is any other pseudouridine-like nucleoside known in the art.

In other embodiments, the nucleoside that is modified in the nucleoside-modified RNA the present disclosure is uridine (U). In other embodiments, the modified nucleoside is cytidine (C). In other embodiments, the modified nucleoside is adenosine (A). In other embodiments the modified nucleoside is guanosine (G).

In other embodiments, the modified nucleoside of the present disclosure is m⁵C (5-methylcytidine). In other embodiments, the modified nucleoside is m⁵U (5-methyluridine). In other embodiments, the modified nucleoside is m⁶A (N⁶-methyladenosine). In other embodiments, the modified nucleoside is s²U (2-thiouridine). In other embodiments, the modified nucleoside is Ψ (pseudouridine). In other embodiments, the modified nucleoside is Um (2′-O-methyluridine).

In other embodiments, the modified nucleoside is m¹A (1-methyladenosine); m²A (2-methyladenosine); Am (2′-O-methyladenosine); ms²m⁶A (2-methylthio-N⁶-methyladenosine); i⁶A (N⁶-isopentenyladenosine); ms²¹6A (2-methylthio-N⁶isopentenyladenosine); io⁶A (N⁶-(cis-hydroxyisopentenyl)adenosine); ms²io⁶A (2-methylthio-N⁶-(cis-hydroxyisopentenyl) adenosine); g⁶A (N⁶-glycinylcarbamoyladenosine); t⁶A (N⁶-threonylcarbamoyladenosine); ms²t⁶A (2-methylthio-N⁶-threonyl carbamoyladenosine); m⁶t⁶A (N⁶-methyl-N⁶-threonylcarbamoyladenosine); hn⁶A(N⁶-hydroxynorvalylcarbamoyladenosine); ms²hn⁶A (2-methylthio-N⁶-hydroxynorvalyl carbamoyladenosine); Ar(p) (2′-O-ribosyladenosine (phosphate)); I (inosine); m¹I (1-methylinosine); m¹Im (1,2′-O-dimethylinosine); m³C (3-methylcytidine); Cm (2′-O-methylcytidine); s²C (2-thiocytidine); ac⁴C (N⁴-acetylcytidine); f⁵C (5-formylcytidine); m⁵Cm (5,2′-O-dimethylcytidine); ac⁴Cm (N⁴-acetyl-2′-O-methylcytidine); k²C (lysidine); m¹G (1-methylguanosine); m²G (N²-methylguanosine); m⁷G (7-methylguanosine); Gm (2′-O-methylguanosine); m²₂G (N²,N²-dimethylguanosine); m²Gm (N²,2′-O-dimethylguanosine); m²₂Gm (N²,N²,2′-O-trimethylguanosine); Gr(p) (2′-O-ribosylguanosine (phosphate)); yW (wybutosine); o₂yW (peroxywybutosine); OHyW (hydroxywybutosine); OHyW* (undermodified hydroxywybutosine); imG (wyosine); mimG (methylwyosine); Q (queuosine); oQ (epoxyqueuosine); galQ (galactosyl-queuosine); manQ (mannosyl-queuosine); preQ₀ (7-cyano-7-deazaguanosine); preQ₁ (7-aminomethyl-7-deazaguanosine); G (archaeosine); D (dihydrouridine); m⁵Um (5,2′-O-dimethyluridine); s⁴U (4-thiouridine); m⁵s²U (5-methyl-2-thiouridine); s²Um (2-thio-2′-O-methyluridine); acp³U (3-(3-amino-3-carboxypropyl)uridine); ho⁵U (5-hydroxyuridine); mo⁵U (5-methoxyuridine); cmo⁵U (uridine 5-oxyacetic acid); mcmo⁵U (uridine 5-oxyacetic acid methyl ester); chm⁵U (5-(carboxyhydroxymethyl)uridine)); mchm⁵U (5-(carboxyhydroxymethyl)uridine methyl ester); mcm⁵U (5-methoxycarbonylmethyluridine); mcm⁵Um (5-methoxycarbonylmethyl-2′-O-methyluridine); mcm⁵s²U (5-methoxycarbonylmethyl-2-thiouridine); nm⁵s²U (5-aminomethyl-2-thiouridine); mnm⁵U (5-methylaminomethyluridine); mnm⁵s²U (5-methylaminomethyl-2-thiouridine); mnm⁵se²U (5-methylaminomethyl-2-selenouridine); ncm⁵U (5-carbamoylmethyluridine); nCm⁵Um (5-carbamoylmethyl-2′-O-methyluridine); cmnm⁵U (5-carboxymethylaminomethyluridine); cmnm⁵Um (5-carboxymethylaminomethyl-2′-O-methyluridine); cmnm⁵s²U (5-carboxymethylaminomethyl-2-thiouridine); m⁶2A (N⁶,N⁶-dimethyladenosine); Im (2′-O-methylinosine); m⁴C (N⁴-methylcytidine); m⁴Cm (N4,2′-O-dimethylcytidine); hm⁵C (5-hydroxymethylcytidine); m³U (3-methyluridine); cm⁵U (5-carboxymethyluridine); m⁶Am (N⁶,2′-O-dimethyladenosine); m⁶2Am (N⁶,N⁶,O-2′-trimethyladenosine); m^{2,7}G (N²,7-dimethylguanosine); m^{2,2,7}G (N²,N²,7-trimethylguanosine); m³Um (3,2′-O-dimethyluridine); m⁵D (5-methyldihydrouridine); f⁵Cm (5-formyl-2′-O-methylcytidine); m¹Gm (1,2′-O-dimethylguanosine); m¹Am (1,2′-O-dimethyladenosine); τm⁵U (5-taurinomethyluridine); τm⁵s²U (5-taurinomethyl-2-thiouridine)); imG-14 (4-demethylwyosine); imG2 (isowyosine); or ac⁶A (N⁶-acetyladenosine).

In other embodiments, a nucleoside-modified RNA of the present disclosure comprises a combination of 2 or more of the above modifications. In other embodiments, the nucleoside-modified RNA comprises a combination of 3 or more of the above modifications. In other embodiments, the nucleoside-modified RNA comprises a combination of more than 3 of the above modifications.

In other embodiments, between 0.10% and 100% of the residues in the nucleoside-modified of the present disclosure are modified (e.g. either by the presence of pseudouridine or a modified nucleoside base). In other embodiments, 0.1% of the residues are modified. In other embodiments, the fraction of modified residues is 0.2%. In other embodiments, the fraction is 0.3%. In other embodiments, the fraction is 0.4%. In other embodiments, the fraction is 0.5%. In other embodiments, the fraction is 0.6%. In other embodiments, the fraction is 0.8%. In other embodiments, the fraction is 1%. In other embodiments, the fraction is 1.5%. In other embodiments, the fraction is 2%. In other embodiments, the fraction is 2.5%. In other embodiments, the fraction is 3%. In other embodiments, the fraction is 4%. In other embodiments, the fraction is 5%. In other embodiments, the fraction is 6%. In other embodiments, the fraction is 8%. In other embodiments, the fraction is 10%. In other embodiments, the fraction is 12%. In other embodiments, the fraction is 14%. In other embodiments, the fraction is 16%. In other embodiments, the fraction is 18%. In other embodiments, the fraction is 20%. In other embodiments, the fraction is 25%. In other embodiments, the fraction is 30%. In other embodiments, the fraction is 35%. In other embodiments, the fraction is 40%. In other embodiments, the fraction is 45%. In other embodiments, the fraction is 50%. In other embodiments, the fraction is 60%. In other embodiments, the fraction is 70%. In other embodiments, the fraction is 80%. In other embodiments, the fraction is 90%. In other embodiments, the fraction is 100%.

In other embodiments, the fraction is less than 5%. In other embodiments, the fraction is less than 3%. In other embodiments, the fraction is less than 1%. In other embodiments, the fraction is less than 2%. In other embodiments, the fraction is less than 4%. In other embodiments, the fraction is less than 6%. In other embodiments, the fraction is less than 8%. In other embodiments, the fraction is less than 10%. In other embodiments, the fraction is less than 12%. In other embodiments, the fraction is less than 15%. In other embodiments, the fraction is less than 20%. In other embodiments, the fraction is less than 30%. In other embodiments, the fraction is less than 40%. In other embodiments, the fraction is less than 50%. In other embodiments, the fraction is less than 60%. In other embodiments, the fraction is less than 70%.

In other embodiments, 0.1% of the residues of a given nucleoside (i.e., uridine, cytidine, guanosine, or adenosine) are modified. In other embodiments, the fraction of the given nucleotide that is modified is 0.2%. In other embodiments, the fraction is 0.3%. In other embodiments, the fraction is 0.4%. In other embodiments, the fraction is 0.5%. In other embodiments, the fraction is 0.6%. In other embodiments, the fraction is 0.8%. In other embodiments, the fraction is 1%. In other embodiments, the fraction is 1.5%. In other embodiments, the fraction is 2%. In other embodiments, the fraction is 2.5%. In other embodiments, the fraction is 3%. In other embodiments, the fraction is 4%. In other embodiments, the fraction is 5%. In other embodiments, the fraction is 6%. In other embodiments, the fraction is 8%. In other embodiments, the fraction is 10%. In other embodiments, the fraction is 12%. In other embodiments, the fraction is 14%. In other embodiments, the fraction is 16%. In other embodiments, the fraction is 18%. In other embodiments, the fraction is 20%. In other embodiments, the fraction is 25%. In other embodiments, the fraction is 30%. In other embodiments, the fraction is 35%. In other embodiments, the fraction is 40%. In other embodiments, the fraction is 45%. In other embodiments, the fraction is 50%. In other embodiments, the fraction is 60%. In other embodiments, the fraction is 70%. In other embodiments, the fraction is 80%. In other embodiments, the fraction is 90%. In other embodiments, the fraction is 100%.

In other embodiments, the fraction of the given nucleotide that is modified is less than 8%. In other embodiments, the fraction is less than 10%. In other embodiments, the fraction is less than 5%. In other embodiments, the fraction is less than 3%. In other embodiments, the fraction is less than 1%. In other embodiments, the fraction is less than 2%. In other embodiments, the fraction is less than 4%. In other embodiments, the fraction is less than 6%. In other embodiments, the fraction is less than 12%. In other embodiments, the fraction is less than 15%. In other embodiments, the fraction is less than 20%. In other embodiments, the fraction is less than 30%. In other embodiments, the fraction is less than 40%. In other embodiments, the fraction is less than 50%. In other embodiments, the fraction is less than 60%. In other embodiments, the fraction is less than 70%.

In other embodiments, a nucleoside-modified RNA of the present disclosure is translated in the cell more efficiently than an unmodified RNA molecule with the same sequence. In other embodiments, the nucleoside-modified RNA exhibits enhanced ability to be translated by a target cell. In other embodiments, translation is enhanced by a factor of 2-fold relative to its unmodified counterpart. In other embodiments, translation is enhanced by a 3-fold factor. In other embodiments, translation is enhanced by a 5-fold factor. In other embodiments, translation is enhanced by a 7-fold factor. In other embodiments, translation is enhanced by a 10-fold factor. In other embodiments, translation is enhanced by a 15-fold factor. In other embodiments, translation is enhanced by a 20-fold factor. In other embodiments, translation is enhanced by a 50-fold factor. In other embodiments, translation is enhanced by a 100-fold factor. In other embodiments, translation is enhanced by a 200-fold factor. In other embodiments, translation is enhanced by a 500-fold factor. In other embodiments, translation is enhanced by a 1000-fold factor. In other embodiments, translation is enhanced by a 2000-fold factor. In other embodiments, the factor is 10-1000-fold. In other embodiments, the factor is 10-100-fold. In other embodiments, the factor is 10-200-fold. In other embodiments, the factor is 10-300-fold. In other embodiments, the factor is 10-500-fold. In other embodiments, the factor is 20-1000-fold. In other embodiments, the factor is 30-1000-fold. In other embodiments, the factor is 50-1000-fold. In other embodiments, the factor is 100-1000-fold. In other embodiments, the factor is 200-1000-fold. In other embodiments, translation is enhanced by any other significant amount or range of amounts.

In other embodiments, the nucleoside-modified antigen-encoding RNA of the present disclosure induces significantly more adaptive immune response than an unmodified in vitro-synthesized RNA molecule with the same sequence. In other embodiments, the modified RNA molecule exhibits an adaptive immune response that is 2-fold greater than its unmodified counterpart. In other embodiments, the adaptive immune response is increased by a 3-fold factor. In other embodiments the adaptive immune response is increased by a 5-fold factor. In other embodiments, the adaptive immune response is increased by a 7-fold factor. In other embodiments, the adaptive immune response is increased by a 10-fold factor. In other embodiments, the adaptive immune response is increased by a 15-fold factor. In other embodiments the adaptive immune response is increased by a 20-fold factor. In other embodiments, the adaptive immune response is increased by a 50-fold factor. In other embodiments, the adaptive immune response is increased by a 100-fold factor. In other embodiments, the adaptive immune response is increased by a 200-fold factor. In other embodiments, the adaptive immune response is increased by a 500-fold factor. In other embodiments, the adaptive immune response is increased by a 1000-fold factor. In other embodiments, the adaptive immune response is increased by a 2000-fold factor. In other embodiments, the adaptive immune response is increased by another fold difference.

In other embodiments, “induces significantly more adaptive immune response” refers to a detectable increase in an adaptive immune response. In other embodiments, the term refers to a fold increase in the adaptive immune response (e.g., 1 of the fold increases enumerated above). In other embodiments, the term refers to an increase such that the nucleoside-modified RNA can be administered at a lower dose or frequency than an unmodified RNA molecule with the same species while still inducing an effective adaptive immune response. In other embodiments, the increase is such that the nucleoside-modified RNA can be administered using a single dose to induce an effective adaptive immune response.

In other embodiments, the nucleoside-modified RNA of the present disclosure exhibits significantly less innate immunogenicity than an unmodified in vitro-synthesized RNA molecule with the same sequence. In other embodiments, the modified RNA molecule exhibits an innate immune response that is 2-fold less than its unmodified counterpart. In other embodiments, innate immunogenicity is reduced by a 3-fold factor. In other embodiments, innate immunogenicity is reduced by a 5-fold factor. In other embodiments, innate immunogenicity is reduced by a 7-fold factor. In other embodiments, innate immunogenicity is reduced by a 10-fold factor. In other embodiments, innate immunogenicity is reduced by a 15-fold factor. In other embodiments, innate immunogenicity is reduced by a 20-fold factor. In other embodiments, innate immunogenicity is reduced by a 50-fold factor. In other embodiments, innate immunogenicity is reduced by a 100-fold factor. In other embodiments, innate immunogenicity is reduced by a 200-fold factor. In other embodiments, innate immunogenicity is reduced by a 500-fold factor. In other embodiments, innate immunogenicity is reduced by a 1000-fold factor. In other embodiments, innate immunogenicity is reduced by a 2000-fold factor. In other embodiments, innate immunogenicity is reduced by another fold difference.

In other embodiments, “exhibits significantly less innate immunogenicity” refers to a detectable decrease in innate immunogenicity. In other embodiments, the term refers to a fold decrease in innate immunogenicity (e.g., 1 of the fold decreases enumerated above). In other embodiments, the term refers to a decrease such that an effective amount of the nucleoside-modified RNA can be administered without triggering a detectable innate immune response. In other embodiments, the term refers to a decrease such that the nucleoside-modified RNA can be repeatedly administered without eliciting an innate immune response sufficient to detectably reduce production of the recombinant protein. In other embodiments, the decrease is such that the nucleoside-modified RNA can be repeatedly administered without eliciting an innate immune response sufficient to eliminate detectable production of the recombinant protein.

### Polypeptide Therapeutic Agents

In other related aspects, the therapeutic agent includes an isolated peptide that modulates a target. For example, In certain embodiments, the peptide of the disclosure inhibits or activates a target directly by binding to the target thereby modulating the normal functional activity of the target. In certain embodiments, the peptide of the disclosure modulates the target by competing with endogenous proteins. In certain embodiments, the peptide of the disclosure modulates the activity of the target by acting as a transdominant negative mutant.

The variants of the polypeptide therapeutic agents may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the polypeptide is an alternative splice variant of the polypeptide of the present disclosure, (iv) fragments of the polypeptides and/or (v) one in which the polypeptide is fused with another polypeptide, such as a leader or secretory sequence or a sequence which is employed for purification (for example, His-tag) or for detection (for example, Sv5 epitope tag). The fragments include polypeptides generated via proteolytic cleavage (including multi-site proteolysis) of an original sequence. Variants may be post-translationally, or chemically modified. Such variants are deemed to be within the scope of those skilled in the art from the teaching herein.

### Antibody Therapeutic Agents

The disclosure also contemplates a delivery vehicle comprising an antibody, or antibody fragment, specific for a target. That is, the antibody can inhibit a target to provide a beneficial effect.

The antibodies may be intact monoclonal or polyclonal antibodies, and immunologically active fragments (e.g., a Fab or (Fab)₂ fragment), an antibody heavy chain, an antibody light chain, humanized antibodies, a genetically engineered single chain FV molecule (Ladner et al, U.S. Pat. No. 4,946,778), or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art.

Antibodies can be prepared using intact polypeptides or fragments containing an immunizing antigen of interest. The polypeptide or oligopeptide used to immunize an animal may be obtained from the translation of RNA or synthesized chemically and can be conjugated to a carrier protein, if desired. Suitable carriers that may be chemically coupled to peptides include bovine serum albumin and thyroglobulin, keyhole limpet hemocyanin. The coupled polypeptide may then be used to immunize the animal (e.g., a mouse, a rat, or a rabbit).

### Combinations

In certain embodiments, the composition of the present disclosure comprises a combination of agents described herein. In certain embodiments, a composition comprising a combination of agents described herein has an additive effect, wherein the overall effect of the combination is approximately equal to the sum of the effects of each individual agent. In other embodiments, a composition comprising a combination of agents described herein has a synergistic effect, wherein the overall effect of the combination is greater than the sum of the effects of each individual agent.

A composition comprising a combination of agents comprises individual agents in any suitable ratio. For example, In certain embodiments, the composition comprises a 1:1 ratio of two individual agents. However, the combination is not limited to any particular ratio. Rather any ratio that is shown to be effective is encompassed.

### Conjugation

In various embodiments of the disclosure, the delivery vehicle is conjugated to a targeting domain. Exemplary methods of conjugation can include, but are not limited to, covalent bonds, electrostatic interactions, and hydrophobic (“van der Waals”) interactions. In certain embodiments, the conjugation is a reversible conjugation, such that the delivery vehicle can be disassociated from the targeting domain upon exposure to certain conditions or chemical agents. In other embodiments, the conjugation is an irreversible conjugation, such that under normal conditions the delivery vehicle does not dissociate from the targeting domain.

In some embodiments, the conjugation comprises a covalent bond between an activated polymer conjugated lipid and the targeting domain. The term “activated polymer conjugated lipid” refers to a molecule comprising a lipid portion and a polymer portion that has been activated via functionalization of a polymer conjugated lipid with a first coupling group. In certain embodiments, the activated polymer conjugated lipid comprises a first coupling group capable of reacting with a second coupling group. In certain embodiments, the activated polymer conjugated lipid is an activated pegylated lipid. In certain embodiments, the first coupling group is bound to the lipid portion of the pegylated lipid. In other embodiments, the first coupling group is bound to the polyethylene glycol portion of the pegylated lipid. In certain embodiments, the second functional group is covalently attached to the targeting domain.

The first coupling group and second coupling group can be any functional groups known to those of skill in the art to together form a covalent bond, for example under mild reaction conditions or physiological conditions. In some embodiments, the first coupling group or second coupling group are selected from the group consisting of maleimides, N-hydroxysuccinimide (NHS) esters, carbodiimides, hydrazide, pentafluorophenyl (PFP) esters, phosphines, hydroxymethyl phosphines, psoralen, imidoesters, pyridyl disulfide, isocyanates, vinyl sulfones, alpha-haloacetyls, aryl azides, acyl azides, alkyl azides, diazirines, benzophenone, epoxides, carbonates, anhydrides, sulfonyl chlorides, cyclooctyne, aldehydes, and sulfhydryl groups. In some embodiments, the first coupling group or second coupling group is selected from the group consisiting of free amines (—NH₂), free sulfhydryl groups (—SH), free hydroxide groups (—OH), carboxylates, hydrazides, and alkoxyamines. In some embodiments, the first coupling group is a functional group that is reactive toward sulfhydryl groups, such as maleimide, pyridyl disulfide, or a haloacetyl. In certain embodiments, the first coupling group is a maleimide.

In certain embodiments, the second coupling group is a sulfhydryl group. The sulfhydryl group can be installed on the targeting domain using any method known to those of skill in the art. In certain embodiments, the sulfhydryl group is present on a free cysteine residue. In certain embodiments, the sulfhydryl group is revealed via reduction of a disulfide on the targeting domain, such as through reaction with 2-mercaptoethylamine. In certain embodiments, the sulfhydryl group is installed via a chemical reaction, such as the reaction between a free amine and 2-iminothilane or N-succinimidyl S-acetylthioacetate (SATA).

In some embodiments, the polymer conjugated lipid and targeting domain are functionalized with groups used in “click” chemistry. Bioorthogonal “click” chemistry comprises the reaction between a functional group with a 1,3-dipole, such as an azide, a nitrile oxide, a nitrone, an isocyanide, and the link, with an alkene or an alkyne dipolarophiles. Exemplary dipolarophiles include any strained cycloalkenes and cycloalkynes known to those of skill in the art, including, but not limited to, cyclooctynes, dibenzocyclooctynes, monofluorinated cyclcooctynes, difluorinated cyclooctynes, and biarylazacyclooctynone

### EXPERIMENTAL EXAMPLES

Various embodiments of the present application can be better understood by reference to the following Examples which are offered by way of illustration. Without further description, it is believed that one of ordinary skill in the art can, using the preceding description and the following illustrative examples, make and utilize the present disclosure and practice the claimed methods. The scope of the present application is not limited to the Examples given herein.

### Materials and Methods

### Ex Vivo Stability Assay

In some embodiments, the disclosure comprises a method of identifying a LNP particle that has increased stability in amniotic fluid. In some embodiments, the LNP is suitable for in utero administration. Generally, the method includes evaluating the stability of at least one LNP using dynamic light scattering in two or more different percentages of a test fluid. Thus, in some embodiments, the method includes the steps of: generating at least one LNP to be tested, contacting an LNP to be tested with a first concentration of a representative fluid of a target subject (e.g., human, mouse, dog), determining the size and polydispersity index (PDI) of the LNP using dynamic light scattering (DLS); contacting an LNP to be tested with at least one additional concentration of the representative fluid of a target subject (e.g., human, mouse, dog), determining the size and polydispersity index (PDI) of the LNP using dynamic light scattering (DLS); comparing the size and polydispersity index (PDI) of the LNP at the at least two fluid concentrations; and identifying the test LNP as having stability in the test fluid based on the changes in size and polydispersity index (PDI) of the LNP. In certain embodiments, a test LNP is identified as having stability in the fluid based on a low level of PDI change between different concentrations of the fluid. In various embodiments, the assay can be utilized to identify LNPs that are stable in amniotic fluid.

Other methods, as well as variations of the methods disclosed herein, will be apparent from the description of this disclosure. In various embodiments, the test LNP concentration in the screening assay can be fixed or varied. A single test LNP, or a plurality of test LNPs, can be tested at one time. In some embodiments, the stability of a test LNP comprising a therapeutic agent can be tested. Suitable therapeutic agents that may be used include, but are not limited to, proteins, nucleic acids, antisense nucleic acids, small molecules, antibodies and peptides.

In certain embodiments, high throughput screening methods involve providing a library containing a large number of test LNPs potentially having the desired stability. Such libraries are then screened in one or more assays, as described herein, to identify those library members that display at least one desired indicator of stability. In some embodiments, the at least one desired indicator of stability is a lower level of change in size, PDI, or a combination of size and PDI between two or more different concentrations of fluid as compared to a control LNP. The test LNPs thus identified can serve as conventional “lead compounds” for further optimization or can themselves be used as delivery vehicles.

Polyamine-Lipid Synthesis and mRNA Production

Ionizable lipid B-4 was prepared via Michael addition chemistry (Kauffman et al., 2015, Nano Lett. 15:7300-7306). Briefly, the polyamine core (Enamine Inc., Monmouth Junction, NJ) was combined with excess lipid epoxide epoxytetradecane (C14) (Sigma-Aldrich, St. Louis, MO) in a 4 mL glass scintillation vial under gentle stirring with a magnetic stir bar for 2 days at 80° C. The reaction mixture was dried using a Rotovap R-300 (Buchi, New Castle, DE) and used for LNP formulation.

The firefly luciferase gene sequence was codon optimized, synthetized, and cloned into an mRNA production plasmid. The m¹Ψ UTP nucleoside modified Fluc mRNA was co-transcriptionally capped using the trinucleotide cap1 analogue (TriLink), and engineered to contain 101 nucleotide-long poly(A) tail. Transcription was performed using MegaScript T7 RNA polymerase and mRNA was precipitated using lithium chloride and purified by cellulose chromatography (Baiersdorfer et al., 2019, Mol. Ther. Nucleic Acids. 15:26-35). The produced mRNAs were analyzed by agarose gel electrophoresis, sequenced, subjected to a standard J2 dot blot, assayed for INF induction in human monocyte derived dendritic cells, and stored frozen at −80° C. for future use.

### LNP Formulation

LNPs were formulated using a 10:1 weight ratio of ionizable lipid B-4 to luciferase mRNA (Kauffman et al., 2015, Nano Lett. 15:7300-7306). First, ionizable lipid B-4 was combined in an ethanol phase with cholesterol (Sigma-Aldrich), 1,2-dioleoyl-sn-glycero-3-phosphoethanolamine (DOPE, Avanti, Alabaster, AL) and 1,2-dimyristoyl-sn-glycero-3-phosphoethanolamine-N-[methoxy(polyethylene glycol)-2000] (ammonium salt) (C14-PEG2000, Avanti) at varying molar ratios (Table 1) to a total volume of 112.5 μL. A separate aqueous phase was prepared with 25 μg of luciferase mRNA in 10 mM citrate buffer (pH=3) to a total volume of 337.5 μL. With a syringe pump, the ethanol and aqueous phases were combined to form LNPs via chaotic mixing using a microfluidic device designed with herringbone features (Chen et al., 2012, J Am Chem Soc. 134(16):6948-6951). LNPs were dialyzed against 1×PBS with a molecular weight cutoff of 20 kDa for 2 hours, sterilized using a 0.22 μm filter, and stored at 4° C. for later use. All materials were prepared and handled ribonuclease-free throughout synthesis, formulation, and characterization steps.

TABLE 1
LNP library formulations including the molar
ratio and molar percentage of excipients.

| | Molar Ratios | | | | Molar Percentage (%) | | | |
|---|---|---|---|---|---|---|---|---|
| Name | B-4 | DOPE | Chol | PEG | B-4 | DOPE | Chol | PEG |
| A1 | 15 | 10 | 5 | 0.5 | 49.18 | 32.79 | 16.39 | 1.64 |
| A2 | 15 | 20 | 20 | 4.5 | 25.21 | 33.61 | 33.61 | 7.56 |
| A3 | 15 | 30 | 35 | 8.5 | 16.95 | 33.90 | 39.55 | 9.60 |
| A4 | 15 | 40 | 50 | 12.5 | 12.77 | 34.04 | 42.55 | 10.64 |
| A5 | 25 | 10 | 20 | 8.5 | 39.37 | 15.75 | 31.50 | 13.39 |
| A6 | 25 | 20 | 5 | 12.5 | 40.00 | 32.00 | 8.00 | 20.00 |
| A7 | 25 | 30 | 50 | 0.5 | 23.70 | 28.44 | 47.39 | 0.47 |
| A8 | 25 | 40 | 35 | 4.5 | 23.92 | 38.28 | 33.49 | 4.31 |
| A9 | 35 | 10 | 35 | 12.5 | 37.84 | 10.81 | 37.84 | 13.51 |
| A10 | 35 | 20 | 50 | 8.5 | 30.84 | 17.62 | 44.05 | 7.49 |
| A11 | 35 | 30 | 5 | 4.5 | 46.98 | 40.27 | 6.71 | 6.04 |
| A12 | 35 | 40 | 20 | 0.5 | 36.65 | 41.88 | 20.94 | 0.52 |
| A13 | 45 | 10 | 50 | 4.5 | 41.10 | 9.13 | 45.66 | 4.11 |
| A14 | 45 | 20 | 35 | 0.5 | 44.78 | 19.90 | 34.83 | 0.50 |
| A15 | 45 | 30 | 20 | 12.5 | 41.86 | 27.91 | 18.60 | 11.63 |
| A16 | 45 | 40 | 5 | 8.5 | 45.69 | 40.61 | 5.08 | 8.63 |

### Dynamic Light Scattering and Zeta Potential

For baseline dynamic light scattering (DLS) measurements, 10 μL of each LNP solution was diluted 100×in 1×PBS in 4 mL disposable cuvettes. For baseline zeta potential measurements, 20 μL of each LNP solution was diluted 50×in deionized water in DTS1070 zeta potential cuvettes (Malvern Panalytical, Malvern, UK). Four measurements each with at least 10 runs were recorded for each sample using a Zetasizer Nano (Malvern Instruments, Malvern, UK). Data are reported as mean±standard deviation (n=3 to 4 measurements).

### LNP pKa Measurements

Surface ionization measurements to calculate the pKa of each LNP formulation were performed as previously described (Hajj et al., 2019, Small, 15:1805097). Buffered solution containing 150 mM sodium chloride, 20 mM sodium phosphate, 20 mM ammonium acetate, and 25 mM ammonium citrate was adjusted to pH 2 to 12 in increments of 0.5. 125 μL of each pH-adjusted solution and 5 μL of each LNP formulation were plated in triplicate in black 96-well plates. 6-(p-toluidinyl)naphthalene-2-sulfonic acid (TNS) was then added to each well to a final TNS concentration of 6 μM. The fluorescence intensity was read on an Infinite 200 Pro plate reader (Tecan, Morrisville, NC) at an excitation wavelength of 322 nm and an emission wavelength of 431 nm. Using least squares regression, the pKa was taken as the pH corresponding to half-maximum fluorescence intensity, i.e., 50% protonation.

LNP Encapsulation Efficiency mRNA encapsulation efficiencies of each LNP formulation were calculated using the Quant-iT-RiboGreen (Thermo Fisher Scientific, Waltham, MA) assay (Heyes et al., 2005, J. Controlled Release. 107:276-287). Each LNP sample was diluted to approximately 2 ng/pL in two microcentrifuge tubes containing 1×TE buffer or 0.1% (v/v) Triton X-100 (Sigma-Aldrich). After 20 min, LNPs in TE buffer and Triton X-100 as well as mRNA standards were plated in triplicate in black 96-well plates and the fluorescent RiboGreen reagent was added per manufacturer's instructions. Fluorescence intensity was read on an Infinite 200 Pro plate reader (Tecan) at an excitation wavelength of 480 nm and an emission wavelength of 520 nm. RNA content was estimated by comparison to a standard curve estimated using least squares linear regression (LSLR). Encapsulation efficiency was calculated as (B-A)/B-100 where A is the RNA content in TE buffer and B is the RNA content in Triton X-100. Encapsulation efficiencies are reported as mean±standard deviation (n=3).

### Animal Experiments

Balb/c (stock #000651) mice were purchased from The Jackson Laboratory (Bar Harbor, ME) and housed in the Laboratory Animal Facility of the Colket Translational Research Building at CHOP. Females of breeding age were paired with males and separated at 24 h to achieve time-dated pregnant dams for amniotic fluid collections or in utero LNP injections as described below. Time-dated Suffolk ewes were obtained from MacCauley Suffolks (Atglen, PA) and time-dated miniature Yucatan swine were obtained from Sinclair Bio Resources (Auxvasse, MO).

### Fluid Collection

For murine serum collections, 8-week-old female C57BL/6 mice (Jackson Laboratory, 18-21 g) were subjected to tail vein blood collections. Blood was centrifuged at 10,000 g for 10 min and the serum supernatant was collected and the cell pellet discarded.

For murine amniotic fluid collections, time-dated Balb/c dams were sacrificed at gestational day 16 (E16), and under sterile conditions a midline laparotomy was performed and the uterine horn was removed. A 27 gauge needle was used to aspirate amniotic fluid from each individual amniotic sac and amniotic fluid was stored at −80° C. For one biological replicate, amniotic fluid was pooled from several fetuses and the volume of amniotic fluid obtained from the amniotic cavity of a single fetus was approximately 100 μL.

For sheep amniotic fluid collection, a time-dated ewe at gestational day 110 (term is approximately 145 days) was anesthetized with 15 mg/kg of intramuscular ketamine with maintenance of general anesthesia using inhaled isoflurane (2-4% in O₂) and propofol (0.2 to 1 mg/kg/min). Intraoperative monitoring included pulse oximetry and constant infusion of isotonic saline administered via a central venous line placed in a jugular vein. Under sterile conditions, a lower midline laparotomy was performed, and the uterus was exposed. A small hysterotomy was then performed and the amniotic fluid was aspirated in a 60 mL syringe and stored at −80° C. until use.

For pig amniotic fluid collection, a time-dated sow at gestational day 100 (term is approximately 114 days) was anesthetized with intramuscular ketamine and acepromazine with maintenance of general anesthesia using inhaled isoflurane and propofol. Intraoperative monitoring included pulse oximetry and constant infusion of isotonic saline administered via a central venous line placed in a jugular vein. Under sterile conditions, a midline laparotomy was performed, the uterus was exposed, and amniotic fluid was aspirated via a 60 mL syringe via a small hysterotomy.

For human specimens, amniotic fluid was collected from the amniotic cavity of an approximately 24-week gestation fetus undergoing an open fetal surgical procedure. Specifically, at the time of hysterotomy, amniotic fluid was aspirated in a sterile manner in a 60 mL syringe and subsequently stored at −80° C. until use.

### Amniotic Fluid Characterization

To characterize the fluids used in this study, pH and protein concentration of all five fluids (mouse serum, mouse amniotic fluid, sheep amniotic fluid, pig amniotic fluid, and human amniotic fluid) were measured. Three pH measurements of each fluid were recorded using a glass combination microelectrode (Thermo Fisher Scientific). Using a NanoQuant Plate (Tecan), protein concentration was estimated by measuring absorbance at excitation wavelengths of 260 nm and 280 nm on an Infinite 200 Pro plate reader (Tecan). pH values and protein concentrations are reported as mean±standard deviation (n=3).

### LNP Stability in Mouse Amniotic Fluid

DLS was used to assess LNP stability in mouse amniotic fluid based on previously described assessments of nanoparticle behavior in human serum albumin and fetal bovine serum (FBS) (Schroffenegger et al., 2020, ACS Nano. 14:12708-12718; Malcolm et al., 2018, ACS Nano. 12:187-197). Briefly, a range of mouse amniotic fluid percentages were selected—0%, 25%, 50%, 75%, and 100% (v/v). DLS measurements of the LNP alone and the fluid alone were used for the 0% and 100% fluid percentages, respectively. The other fluid percentages were calculated using the equation

$$A / ( A + B ) \cdot 100$$

where A is the volume of fluid and B is the volume of LNP in 1×PBS. The LNP volume was held constant at 10 μL for all DLS stability measurements. For all fluid percentages, LNPs were incubated in E16 mouse amniotic fluid for 30 minutes at 37° C. under gentle agitation at 300 rpm. After 30 minutes, the entire incubation volume of each sample was diluted 100×in PBS and transferred to a cuvette for DLS measurement.

A range of incubation timepoints was also selected—0 min, 5 min, 15 min, 30 min, 60 min, 120 min, and 240 min. For all time points, LNPs were incubated in 50% (v/v) mouse amniotic fluid at 37° C. under gentle agitation at 300 rpm. Following incubation, the LNP and fluid samples were prepared for DLS as described elsewhere herein.

Both experiments were repeated in triplicate using three biological replicates of E16 mouse amniotic fluid. All DLS readings in the present study involved four independent measurements, each the average of 10 runs. The LNP size described throughout this study is the mean peak intensity diameter (nm) of intensity distribution measurements from DLS. Intensity curves are shown as the mean intensity (n=3 to 4) for each data point as a function of size (nm). Size and polydispersity index (PDI) are reported as the mean±standard deviation (n=3 to 4 measurements per biological replicate).

### LNP Library Stability Screen in Mouse Serum and Mouse, Pig, Sheep and Human Amniotic Fluid

All 16 LNP formulations were evaluated in the five fluids listed elsewhere herein. Due to the precious nature of many of these samples and the reasonable standard deviations of measurements collected with three biological replicates of mouse amniotic fluid, only one biological replicate of each fluid was used in the library screen. Following incubation in 50% (v/v) fluid for 30 minutes at 37° C. with gentle agitation at 300 rpm, the entire incubation volume was diluted 100×in 1×PBS and transferred to a cuvette for DLS measurement.

The percent change in size for each LNP and fluid combination was calculated as:

$$\vert B - A \vert / B \cdot 100$$

where A is the mean LNP size in PBS and B is the LNP size in fluid. Similarly, the percent change in PDI for each LNP and fluid combination was calculated as:

$$\vert D - C \vert / D \cdot 100$$

where C is the mean PDI of the LNP in PBS and D is the PDI of the LNP in fluid.

Percent change in size and percent change in PDI measurements were compared by 2-way ANOVA across fluid type and formulation with the Tukey-Kramer correction for multiple comparisons. Hits were identified as LNP and fluid combinations that had significantly (p<α=0.05) lower percent change in size or percent change in PDI measurements than the same LNP in mouse serum.

An instability parameter was defined to concurrently evaluate the effect of both percent change in size and percent change in PDI on overall LNP stability; it is defined as the mean of the two measurements. LSLR was used to compare mean instability parameter measurements for the LNP library across species for amniotic fluids with goodness of fit quantified by the coefficient of determination R₂.

### TEM and Zeta Potential Characterization of LNPs in Mouse Amniotic Fluid

Transmission electron microscopy (TEM) images were obtained using a JEOL 1010 electron microscope system (Jeol, Tokyo, Japan) operated at 80 kV. LNP samples were deposited on thin carbon films (Ted Pella Inc., Redding, CA) supported by nickel grids and were stained with 2% uranyl acetate (Electron Microscopy Sciences, Hatfield, PA) before observation. For LNP formulations in PBS and with mouse amniotic fluid, the shortest edge to edge diameter of 20 particles was manually measured with ImageJ. The reported diameter is the mean±standard deviation (n=20).

For zeta potential measurements of LNPs in mouse amniotic fluid, LNPs A12 and A1 were incubated for 30 minutes at 37° C. with gentle agitation at 300 rpm in six percentages of mouse amniotic fluid—0%, 10%, 25%, 50%, 75%, and 100% (v/v). Samples were immediately diluted 50×in deionized water and loaded into DTS1070 zeta potential cuvettes (Malvern Panalytical). Zeta potential was measured using a Zetasizer Nano (Malvern Instruments) and measurements are reported as mean±standard deviation (n=3).

### Chromatography and Protein Quantification of LNPs in Mouse Amniotic Fluid

The most (A12) and least stable (A1) LNPs from the mouse amniotic fluid stability screen were incubated in mouse amniotic fluid and isolated from unbound fluid proteins via a Sepharose CL-6B affinity chromatography column. 32 fractions, each equal and approximately 100 μL in volume, were collected following loading of (i) A12 with mouse amniotic fluid, (ii) A1 with mouse amniotic fluid, or (iii) free mouse amniotic fluid. For all three samples, protein concentration was evaluated in each fraction using a NanoQuant Plate (Tecan) and read on an Infinite 200 Pro plate reader (Tecan). Fractions with non-zero protein concentration readings that did not overlap with free mouse amniotic fluid fractions were identified by plotting protein concentration versus fraction number. These identified fractions were pooled and further evaluated for protein content using a micro BCA protein assay kit (Thermo Fisher). Pooled fractions from column separation, LNPs A12 and A1 in PBS, and standard curve samples were incubated at a 1:1 ratio of sample to working reagent at 60° C. for 60 minutes. Following incubation, samples were allowed to cool and plated in triplicate on a 96-well plate. Absorbance at a wavelength of 562 nm was immediately read on an Infinite 200 Pro plate reader (Tecan). The protein concentration was quantified by comparing sample absorbances to a standard curve using LSLR. A paired t test was used to determine significant differences in protein concentration between LNPs in PBS and with mouse amniotic fluid. Protein concentrations are reported as mean±standard deviation (n=3).

In Vitro LNP-mediated Luciferase mRNA Delivery to HeLa Cells

HeLa cells (ATCC no. CCL-2) were cultured in DMEM with L-glutamine (Thermo Fisher Scientific) supplemented with 10% FBS and 1% penicillin-streptomycin. Cells were plated at 10,000 cells per well in 100 μL of medium in tissue culture treated 96-well plates and were left to adhere overnight. All 16 LNP formulations were incubated with 50% (v/v) mouse amniotic fluid for 30 minutes at 37° C. under gentle agitation at 300 rpm. LNP formulations pre-incubated in mouse amniotic fluid or LNPs in PBS alone were used to treat cells at a dose of 50 ng of mRNA per 10,000 cells. As a positive control, the transfection reagent lipofectamine MessengerMAX (Thermo Fisher Scientific) was combined with luciferase mRNA for 10 minutes as per the manufacturer's protocol and was used to treat cells at a dose of 50 ng of mRNA per 10,000 cells. 24 hours after treatment with LNPs and lipofectamine, cells were centrifuged at 300 g for 5 minutes and excess medium was removed. 50 μL of 1×lysis buffer (Promega, Madison, WI) followed by 100 μL of luciferase assay substrate (Promega) was added to each well. After 10 minutes of incubation, luminescence was quantified using an Infinite 200 Pro plate reader (Tecan). The luminescence signal for each condition was normalized by dividing by the luminescence signal from untreated control cells.

To evaluate cytotoxicity, additional plates were prepared as described elsewhere herein. After 24 hours, 100 μL of CellTiter-Glo (Promega) was added to each well and the luminescence corresponding to ATP production was quantified using a plate reader following 10 minutes of incubation. Luminescence for each group was normalized by dividing by the luminescence signal from untreated control cells.

Luciferase expression and percent viability are reported as mean±standard deviation (n=3 biological replicates and at least 2 technical replicates per plate). GraphPad Prism's ROUT method (Motulsky et al., 2006, BMC Bioinformatics. 7:123) with Q=5% was used to identify outliers across treatment conditions and subsequently remove them from mean and standard deviation calculations. For both luciferase expression and percent viability, 2-way ANOVA with the Tukey-Kramer correction for multiple comparisons was used to compare means across formulation and treatment condition.

### In Utero Studies

In utero intra-amniotic injections were performed as previously described (Alapati et al., 2019, Sci. Transl. Med. 11). Briefly, under isoflurane anesthesia and after providing local anesthetic with 0.25% bupivacaine subcutaneously, a midline laparotomy was performed, and the uterine horn was exposed. Under a dissecting microscope, 30 μL of PBS or LNPs concentrated to 325 ng/pL was injected into the amniotic cavity of each fetus using a custom made 80 μm beveled glass micropipette and an automated microinjector (Narishige IM-400 Electric Microinjector, Narishige International USA Inc., Amityville, NY). After successful injection, the uterus was returned to the peritoneal cavity and the abdomen was closed with a single layer of absorbable 4-0 polyglactin 910 suture. A group size of n=5 was used for each of the three treatment groups (LNP A12, LNP A4, and PBS injections).

### Luciferase Imaging and Quantification

Luciferase signal was assessed in treated fetuses as well as individual fetal organs following in utero intra-amniotic injection of LNPs containing luciferase mRNA as described (Riley et al., 2021, Sci. Adv. 7:eaba1028). Specifically, mice were imaged 4 h after intra-amniotic injection of LNPs or PBS. Luciferase imaging was performed using an in vivo imaging system (IVIS, PerkinElmer, Waltham, MA). Ten minutes before sacrifice and imaging, dams were injected intraperitoneally with D-luciferin at 150 mg/kg and potassium salt (Biotium, Fremont, CA). Pregnant dams were then placed supine into the IVIS, and luminescence signal was detected with a 60 s exposure time. Next, a midline laparotomy was performed to expose the uterine horn, and luciferase imaging was repeated. Following imaging of the dam with the uterine horn exposed, fetuses were removed and individually imaged using IVIS with 60 s exposure times. The fetal liver, intestines, lungs, and brain were subsequently removed and imaged by IVIS. Image analysis was conducted using the Living Image software (PerkinElmer). To quantify luminescence flux, a rectangular region of interest (ROI) was placed in an area without any luminescence signal in the same image. Normalized flux was calculated by dividing the total flux from the ROI over the fetus or organ by the total flux from the background ROI. For each treatment group, the ROUT outlier test with Q=1% was used to identify and remove outliers. Reported fetal and organ bioluminescence represent the mean±standard error of the mean (SEM) (n≥4). The representative organ IVIS images shown are those that have the highest luminescence values for each treatment condition.

### Example 1: LNP Library Design, Formulation, and Characterization

To engineer and identify LNPs with optimal stability in amniotic fluid, a library of 16 LNP formulations was designed using an orthogonal design of experiments (DOE) approach. Orthogonal DOE was chosen because it is a well-defined methodology for screening and optimizing nanoparticles, while minimizing the total number of formulations tested.

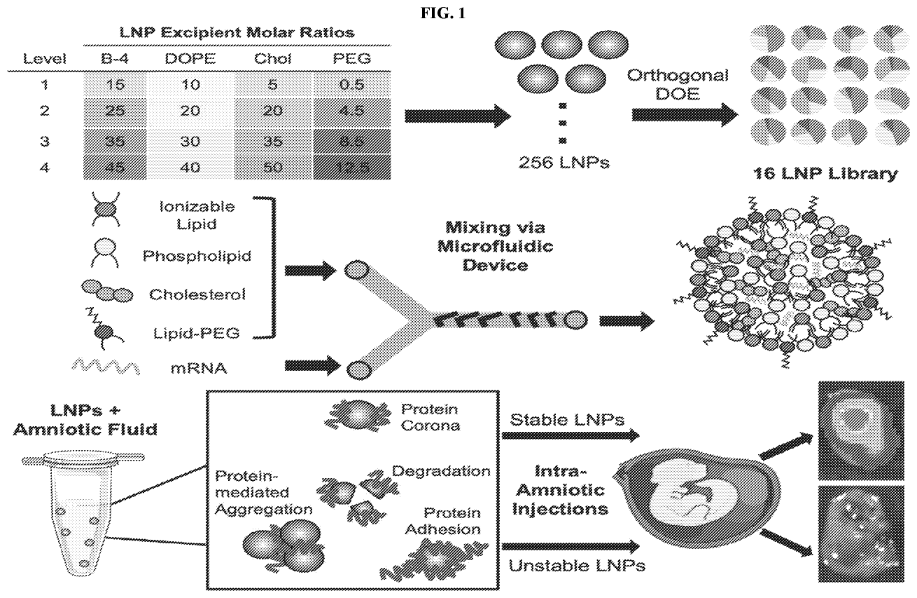

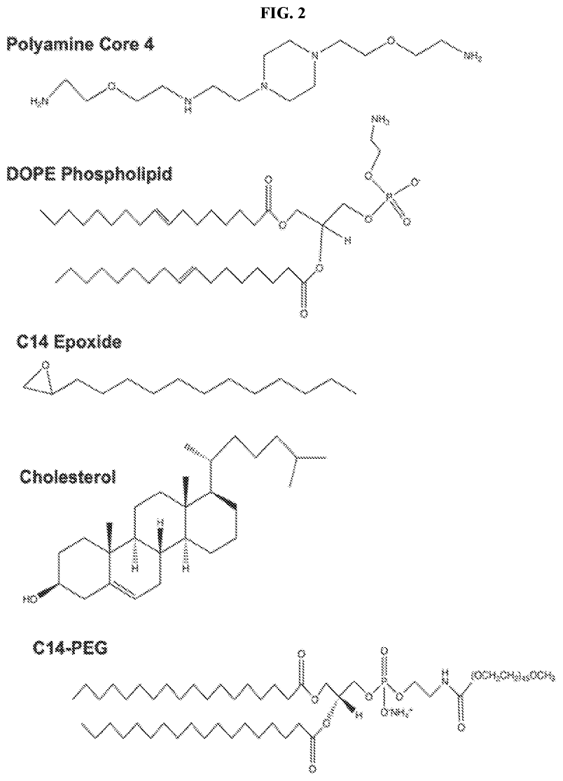

Theoretically, 256 combinations are possible when varying four molar ratios of each of four excipients. However, by using orthogonal design, the effects of the four excipients and their four molar ratios can be evaluated using only 16 formulations (Table 1). Therefore, four excipients at varying molar ratios (FIG. 1) were used to formulate LNPs: (i) an ionizable lipid, (ii) 1,2-dioleoyl-sn-glycero-3-phosphoethanolamine (DOPE), (iii) cholesterol, and (iv) lipid-anchored polyethylene glycol (lipid-PEG) (FIG. 2). The ionizable lipid B-4 was selected based on previously published work demonstrating that LNPs formulated with the B-4 ionizable lipid had the highest fetal lung delivery following vitelline vein injection in gestational age E16 fetuses of pregnant mice (Riley et al., 2021, Sci. Adv. 7:eaba1028).

Fetal lungs are often one main organ target for intra-amniotic administration of gene therapies. DOPE, cholesterol, and lipid-PEG were selected based on previous work indicating their inclusion in LNPs enables efficient delivery of mRNA in adult mice (Oberli et al., 2017, Nano Lett. 17:1326-1335). The phospholipid DOPE promotes LNP membrane formation and endosomal escape, cholesterol enhances membrane stability, and lipid-PEG limits immune system recognition and rapid clearance. Due to the structural impact of these LNP excipients, without being bound by theory, it was hypothesized that screening a range of molar ratios for each of these lipid excipients would impact ex utero LNP stability in amniotic fluids.

Following library design, all 16 LNPs were formulated using the ionizable lipid B-4. As previously described, the ionizable lipid was synthesized using Michael addition chemistry where the polyamine core was reacted with 14-carbon alkyl tails (Kauffman et al., 2015, Nano Lett. 15:7300-7306). B-4 was then mixed in an ethanol phase with the remaining lipid excipients—DOPE, cholesterol, and lipid-PEG—and combined with an aqueous phase of luciferase mRNA via chaotic mixing in a microfluidic device (FIG. 1) (Chen et al., 2012, J Am Chem Soc. 4).

LNPs were characterized by hydrodynamic diameter, polydispersity index (PDI), encapsulation efficiency, pKa, and zeta potential (Table 2). Using intensity measurements from dynamic light scattering (DLS), LNP size ranged from 46 to 153 nm and six out of 16 LNPs had PDIs>0.3. A RiboGreen assay was used to characterize mRNA encapsulation efficiency, and seven out of 16 LNPs had encapsulation efficiencies<75%.

These results indicate that the wide range of molar ratios selected for library design conferred large LNP size, high polydispersity, and low encapsulation efficiency for some formulations. Next, LNPs were characterized by their pKa, or the pH at which the LNP is 50% protonated. pKa is an indicator of the ability of an LNP to escape the acidic environment of the endosome following endocytosis. In the endosome, LNPs with pKa values<7 will become protonated causing their membrane lipids to fuse with the anionic lipid of the endosome, and release their mRNA cargo into the cytosol.

Typically, ionizable LNPs with pKa values from 6 to 7 enable potent delivery of nucleic acids. The measured pKa values for the 16 LNP library ranged from 6.03 to 6.63 indicating that all LNPs were in the optimal range to enable endosomal escape. Finally, zeta potential measurements ranged from −7.4 to 25 mV, and 13 out of 16 LNPs had neutral to positive zeta potential values as expected.

TABLE 2
Characterization of LNP library including size and PDI in PBS, mRNA
concentration, encapsulation efficiency, pKa, and zeta potential

| | | | mRNA | Encapsulation | | Zeta Potential |
|---|---|---|---|---|---|---|
| Name | Size (nm) | PDI | (ng/μL) | Efficiency (%) | pKₐ | (mV) |
| A1 | 137 ± 15 | 0.28 ± 0.03 | 33 ± 3 | 75 ± 2 | 6.63 | 25.0 ± 2.0 |
| A2 | 105 ± 7 | 0.24 ± 0.02 | 31 ± 9 | 84 ± 6 | 6.17 | −0.01 ± 0.8 |
| A3 | 62 ± 11 | 0.37 ± 0.02 | 46 ± 11 | 93 ± 1 | 6.53 | 1.5 ± 0.3 |
| A4 | 95 ± 10 | 0.28 ± 0.02 | 44 ± 26 | 89 ± 7 | 6.52 | 2.4 ± 0.5 |
| A5 | 100 ± 6 | 0.19 ± 0.03 | 34 ± 4 | 53 ± 4 | 6.57 | −7.4 ± 0.6 |
| A6 | 89 ± 3 | 0.18 ± 0.02 | 14 ± 3 | 38 ± 9 | 6.28 | 4.6 ± 0.9 |
| A7 | 88 ± 6 | 0.20 ± 0.01 | 21 ± 0.3 | 93 ± 0.2 | 6.51 | 19.6 ± 0.2 |
| A8 | 46 ± 3 | 0.61 ± 0.05 | 35 ± 3 | 96 ± 0.1 | 6.48 | 15.2 ± 0.8 |
| A9 | 153 ± 6 | 0.22 ± 0.03 | 23 ± 0.1 | 63 ± 0.2 | 6.46 | 7.7 ± 0.3 |
| A10 | 110 ± 13 | 0.30 ± 0.10 | 30 ± 1 | 94 ± 0.2 | 6.46 | 10.2 ± 0.5 |
| A11 | 137 ± 11 | 0.41 ± 0.05 | 31 ± 2 | 69 ± 2 | 6.53 | 6.0 ± 1.0 |
| A12 | 89 ± 5 | 0.25 ± 0.01 | 41 ± 7 | 93 ± 1 | 6.21 | 14.5 ± 0.8 |
| A13 | 148 ± 10 | 0.33 ± 0.07 | 37 ± 6 | 95 ± 0.9 | 6.45 | 11.7 ± 0.7 |
| A14 | 136 ± 9 | 0.28 ± 0.02 | 18 ± 4 | 89 ± 2 | 6.03 | 18.9 ± 0.5 |
| A15 | 108 ± 2 | 0.31 ± 0.03 | 13 ± 3 | 31 ± 5 | 6.52 | −0.9 ± 0.7 |
| A16 | 142 ± 10 | 0.31 ± 0.02 | 7 ± 4 | 16 ± 6 | 6.44 | 3.2 ± 0.9 |

### Example 2: Ex Utero LNP Stability in Mouse Amniotic Fluid

Previous work has reported the stability of lipid-based nanoparticle systems in well-characterized fluids such as serum using DLS (Chen et al., 2019, Nanoscale. 11:8760-8775; Malcolm et al., 2018, ACS Nano. 12:187-197; Colapicchioni et al., 2016, Int. J. Biochem. Cell Biol. 75:180-187). DLS is a minimal resource, quantitative assay for measuring the size distribution and polydispersity of a nanoparticle sample, and the stability of LNPs following ex vivo incubation in a variety of fluids can be assessed using this technique. For example, more stable LNPs will exhibit smaller size and polydispersity changes upon incubation in different fluids, therefore allowing the identification of highly stable and unstable LNPs in each fluid of interest.

To determine the DLS incubation parameters for the ex vivo screening of the LNP library, a range of mouse amniotic fluid percentages were selected—0%, 25%, 50%, 75%, and 100% (v/v)—and a range of incubation times—0 min, 5 min, 15 min, 30 min, 60 min, 120 min, and 240 min—for evaluation. Two LNPs, A5 and A12, were selected for this investigation and mouse amniotic fluid collected from gestational day 16 (E16) fetuses was used. E16 amniotic fluid is representative of the biological environment in the amniotic sac at the onset of fetal breathing and was selected as it represents the timeframe during which intra-amniotic gene therapies could be administered to take advantage of fetal inhalation and ingestion of LNPs from the amniotic fluid for lung and digestive tract delivery.

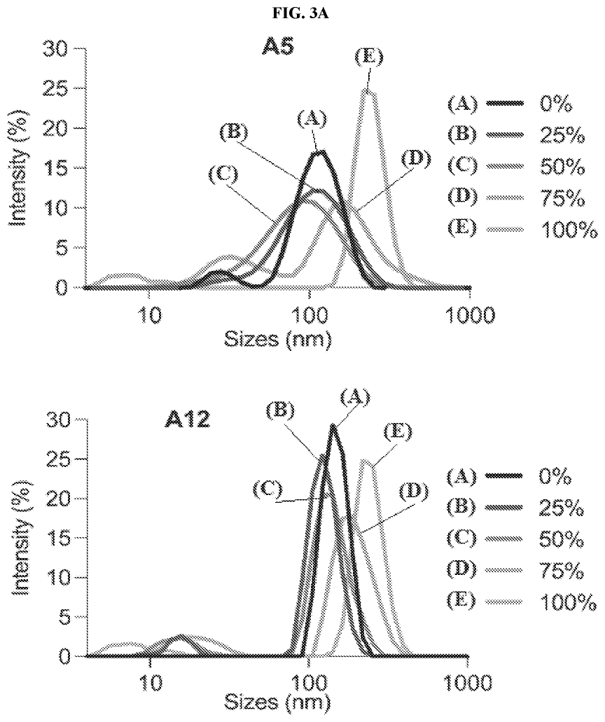

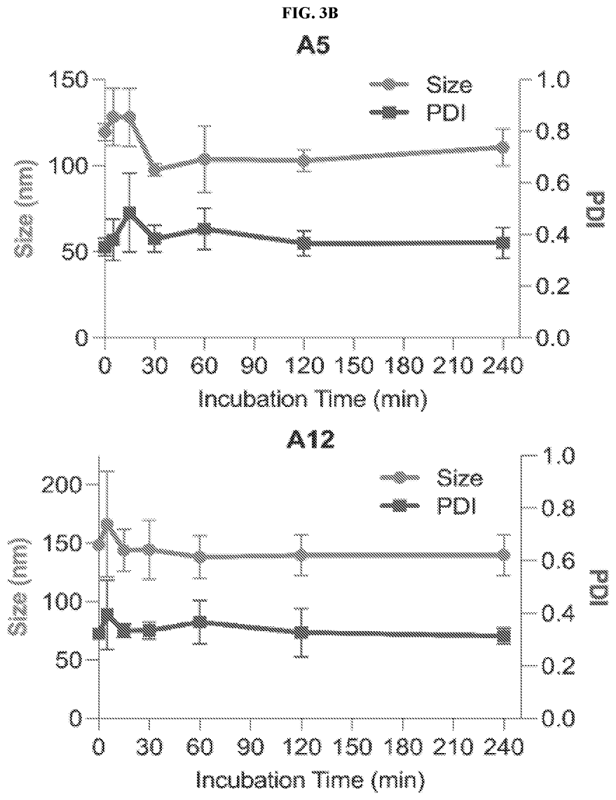

Across a range of amniotic fluid percentages, LNP A5 exhibited broadening of the DLS intensity curve along the x-axis as amniotic fluid percentage increased, until ultimately becoming bimodal in 75% amniotic fluid (FIG. 3A). This suggests that the A5 LNP population became more heterogenous in size with increasing polydispersity as the amniotic fluid percentage increased. In contrast, as the amniotic fluid percentage increased, the A12 intensity curves shifted right along the size axis and began overlapping with the intensity curve for 100% mouse amniotic fluid. This suggests that as the mouse amniotic fluid percentage increased, the mean A12 LNP size increased with little change in polydispersity. In general, for both LNPs, there were less-substantial effects on size and polydispersity in 25% mouse amniotic fluid. Additionally, this low fluid percentage is less physiologically relevant for applications of intra-amniotic injection of LNPs where the particles would be exposed to 100% amniotic fluid in the sac. Alternatively, in 75% mouse amniotic fluid, both LNPs were unstable with either a very high polydispersity or a large increase in size. Therefore, to ensure resolution between stable and unstable LNPs in each of the fluids of interest, 50% (v/v) amniotic fluid was selected for the subsequent library stability screen.

Previous work has evaluated lipid-based nanoparticle stability following incubation in protein-rich fluid for times comparable to 5 and 15 min (Malcolm et al., 2018, ACS Nano. 12:187-197). However, results in the present study indicate that size and PDI measurements from these incubation times had large standard deviations and did not represent longer-term LNP stability in mouse amniotic fluid (FIG. 3B). Instead, there were minimal changes in size and PDI measurements for incubation times greater than or equal to 30 min. Therefore, while the 240 min incubation timepoint was selected to represent the maximum LNP residence time in amniotic fluid before evaluation of in utero delivery, 30 min was selected as the incubation time as it sufficiently represents longer-term behavior of LNPs in mouse amniotic fluid.

### Example 3: LNP Stability in Mouse Serum and Mouse, Large Animal, and Human Amniotic Fluids

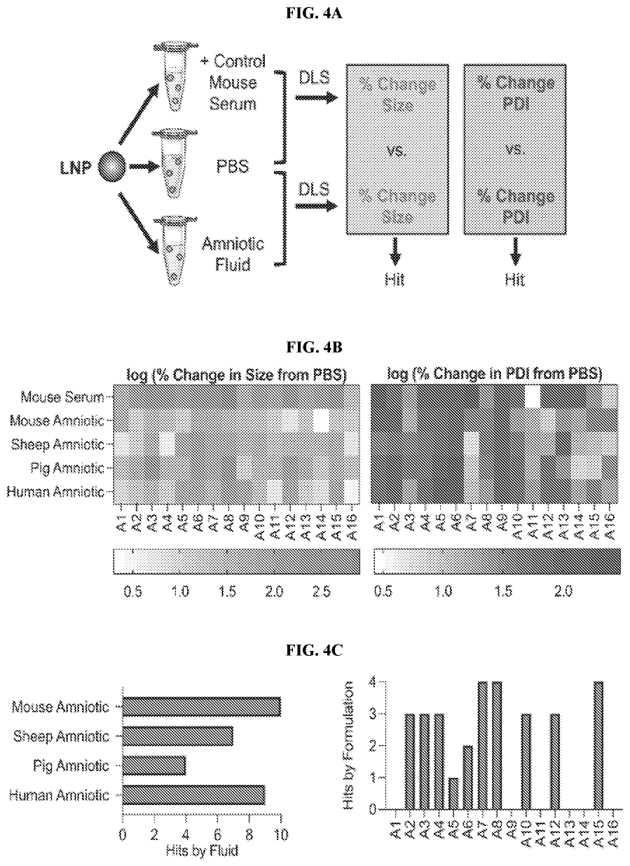

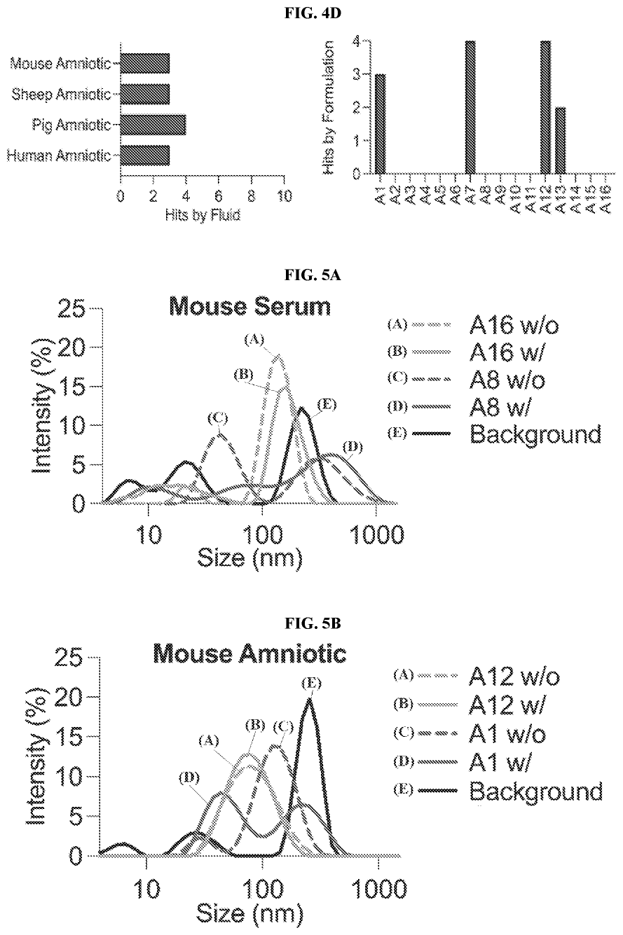

Using DLS, all 16 LNPs were evaluated ex vivo for stability in the following five fluids: mouse serum, mouse amniotic fluid, sheep amniotic fluid, pig amniotic fluid, and human amniotic fluid. Mouse serum was selected as one fluid of interest as numerous prior studies have characterized the stability of lipid-based nanoparticle systems in various blood and serum fluids, including human plasma and fetal bovine serum (FBS). Additionally, as some of these studies report significant lipid nanoparticle instability at low concentrations (1% or 2% v/v) of FBS, without being bound by theory it was hypothesized that mouse serum could serve as a positive control for this screen (FIG. 4A).

Results of the screen were quantified via percent change in size and percent change in PDI (both from the LNP in PBS alone) following incubation in fluid. These percent change parameters were selected to take into account the initial size and PDI of the LNP before incubation, as they varied widely across the library (Table 2). Also, these parameters allow for an intuitive understanding of stability—with stable LNPs having low percent changes in size or PDI measurements following incubation in any given fluid. Therefore, results were presented in a heatmap with the log transforms of the percent change in size and percent change in PDI measurements (FIG. 4B). Log transformation allows a larger range of values to be presented in a color gradated scale, without having very large measurements diminish the resolution present between smaller measurements.

Mouse serum served as a positive control for this library screen since many LNPs performed substantially worse in serum than the other fetal fluids (FIG. 4B). To aid in visualization of the library screen findings, a 2-way ANOVA was performed to define hits, or LNPs in a given fluid whose percent change in size or percent change in PDI measurements were significantly smaller (p<0.05) than the same LNP in mouse serum (FIGS. 4C-4D). First, for percent change in size measurements (FIG. 4C), there were ten and nine LNP hits in mouse amniotic and human amniotic fluids, respectively, while there were only four LNP hits in pig amniotic fluid. Taken together, these results suggest that more LNPs from the library were stable in mouse and human amniotic fluids than in pig amniotic fluid. However, for percent change in PDI measurements (FIG. 4D), all fluids had only between three and four LNP hits, suggesting that the LNP library performed similarly across all fluids.

Percent change in size measurements identified that ten out of 16 LNPs in the library were a hit in at least one fluid (FIG. 4C). Yet, percent change in PDI measurements identified that only four out of 16 LNPs in the library were a hit in at least one fluid (FIG. 4D). Collectively, these results suggest that there were substantially fewer hits for percent change in PDI measurements than percent change in size measurements when compared to mouse serum. In other words, LNPs in a given fluid are more likely to have significantly smaller percent change in size measurements compared to mouse serum than percent change in PDI measurements. It is important to note that LNPs such as A9 and A16 were not identified as hits in any of the fetal fluids. However, this is likely because these LNPs appeared to be stable in mouse serum, as they exhibited low percent change in size and PDI measurements.

Therefore, no significant improvements compared mouse serum in any of the fetal fluids could be identified. While these results are intriguing, LNPs A9 and A16 also had low encapsulation efficiencies (≤75%), therefore limiting their application for mRNA delivery and future exploration in this study.

The majority of LNPs presented both substantial percent change in size and percent change in PDI measurements following incubation in fluid. However, some LNPs appeared to demonstrate mainly high percent change in size measurements (A8), while others presented mainly high percent change in PDI measurements (A9) (FIG. 4B). Therefore, it was rationalized that both parameters should be considered when evaluating overall LNP stability. To determine the most and least stable LNP in each of the fluids, the percent change in size and percent change in PDI measurements were averaged for an overall lowest and highest LNP instability parameter. The top LNPs in each amniotic fluid evaluated were as follows: A12 for mouse amniotic, A14 for pig amniotic, and A16 for sheep and human amniotic. A12 and A14 LNPs had several commonalities: a moderate to high molar ratio of B-4 ionizable lipid (35-45), a low molar ratio of cholesterol (20-35), and a low molar ratio of lipid-PEG (0.5) compared to traditional LNP formulations for mRNA delivery. However, as mentioned above, LNP A16 had an encapsulation efficiency of less than 75%, so this formulation likely would require further optimization for sheep and human intra-amniotic delivery.

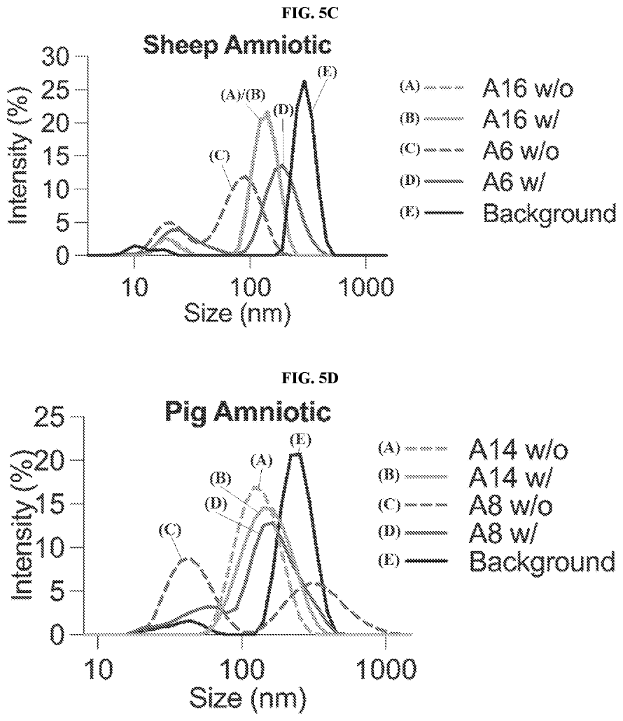

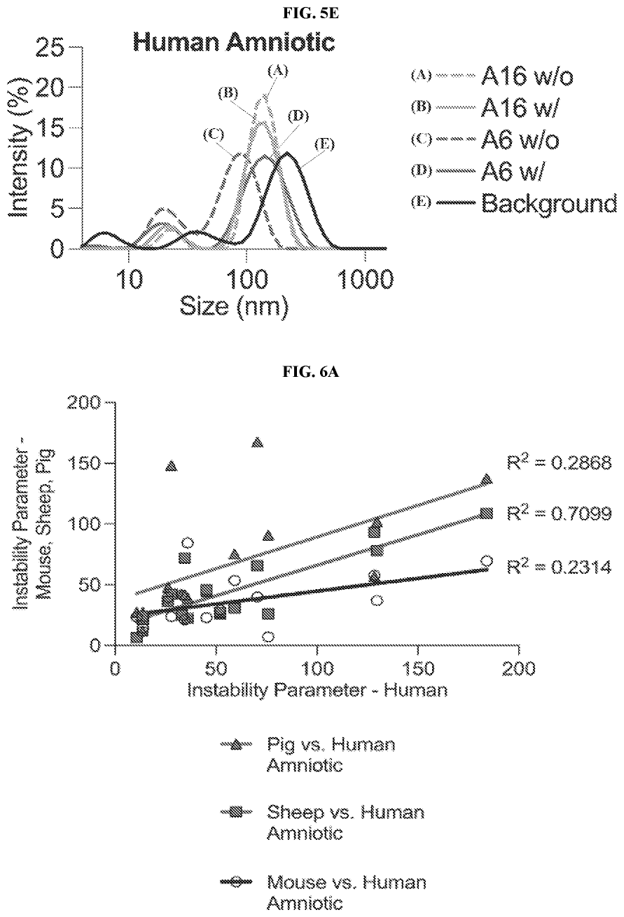

Representative DLS intensity curves (FIGS. 5A-5E) of the most stable LNPs often showed little to no size or PDI change in the presence of amniotic fluid compared to the intensity curve of the LNP in PBS alone. Instead, intensity curves of the least stable LNPs in each of the fluids showed increased PDI, bimodal behavior, and substantial size increases as curves shifted right and sometimes completely overlapped with the intensity curve of the amniotic fluid background.

### Example 4: Stability Correlations in Amniotic Fluid Across Species

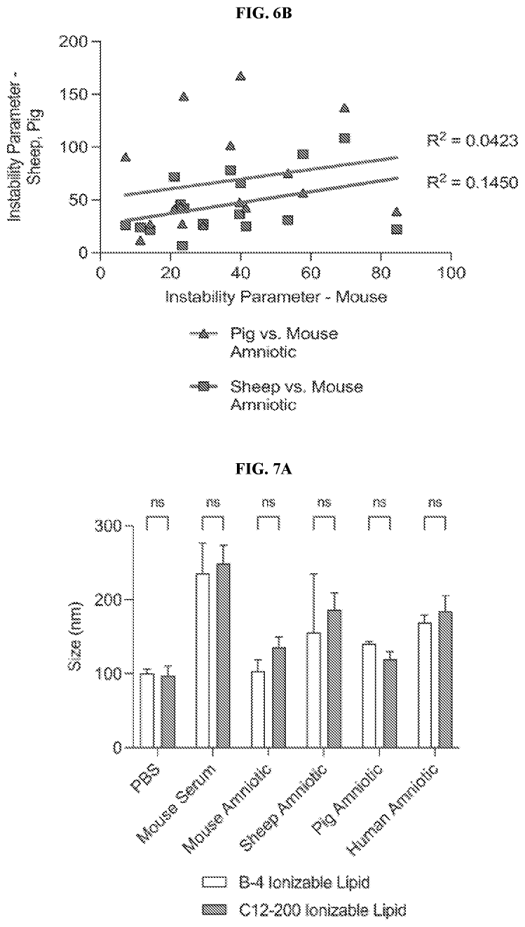

Next, it was investigated whether there was any correlations between LNP stability in amniotic fetal fluids across species using the above defined instability parameter (FIGS. 6A-6B). First, LNP instability parameters in mouse and pig amniotic fluids only mildly correlated with those in human amniotic fluid (R²=0.2314 and R²=0.2868, respectively). However, there was a moderate correlation of the instability parameter measurements between sheep amniotic and human amniotic fluids (R²=0.7099). In terms of stability, these results suggest that LNPs performed most similarly in a sample of sheep amniotic fluid as they did in human amniotic fluid, more so than in samples of mouse and pig amniotic fluids. Next, LNP instability parameters in pig and sheep amniotic fluids had little to no correlation with those in mouse amniotic fluid (R²=0.0423 and R²=0.1450, respectively). These results suggest that mouse amniotic LNP stability may not accurately correlate with stability in amniotic fluid samples of larger species, specifically pig and sheep. Taken together, these results demonstrate differences in LNP stability in amniotic fluids between small animal models and large animal models or humans, perhaps due to gestational age differences at the time of amniotic fluid collection and total length of gestational periods. Finally, while fluids used in this screen were characterized in terms of their pH and protein concentration (Table 3), there were no notable trends between stability measurements of the LNP library and either fluid pH or protein concentration.

TABLE 3
Characterization of mouse serum and
fetal fluids used in library screen.

| Fluid | pH | Protein Concentration (ng/μL) |
|---|---|---|
| Mouse Serum | 8.45 ± 0.10 | 1507 ± 60 |
| Mouse Amniotic | 7.58 ± 0.11 | 144 ± 2 |
| Sheep Amniotic | 8.07 ± 0.13 | 515 ± 12 |
| Pig Amniotic | 7.77 ± 0.09 | 308 ± 9 |
| Human Amniotic | 8.1 ± 0.06 | 140 ± 2 |

### Example 5: Effect of Ionizable Lipid on LNP Stability

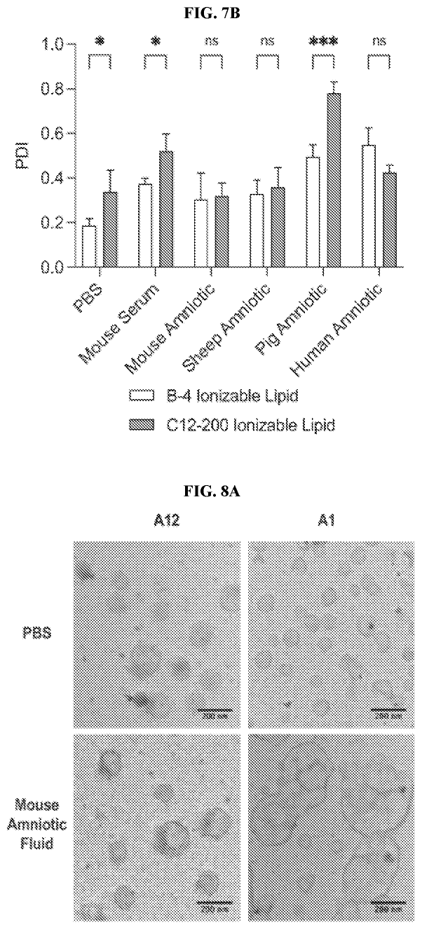

To evaluate the generalizable nature of this ex vivo stability assay across different ionizable lipids, the effect of changing the ionizable lipid in an LNP formulation on stability measurements was evaluated. To this end, excipient formulation A5 was selected and the B-4 ionizable lipid was replaced with C12-200, a well characterized ionizable lipid for mRNA delivery. There were no significant (*p<0.05) differences in the measured size of the B-4 and C12-200 LNPs in any of the fluids evaluated (FIG. 7A). However, in two of the amniotic fluids evaluated, the C12-200 LNP had significantly (*p<0.05 and ***p<0.001) higher PDI measurements than the B-4 LNP (FIG. 7B). Without being bound by theory, it was hypothesized that this difference was due to the significantly higher initial PDI of the C₁₂-200 LNP in PBS alone compared to the B-4 formulation also in PBS. These results suggest the reproducibility of ex vivo stability measurements for formulations with different ionizable lipids.

### Example 6: LNP Morphology and Zeta Potential Effects in Mouse Amniotic Fluid

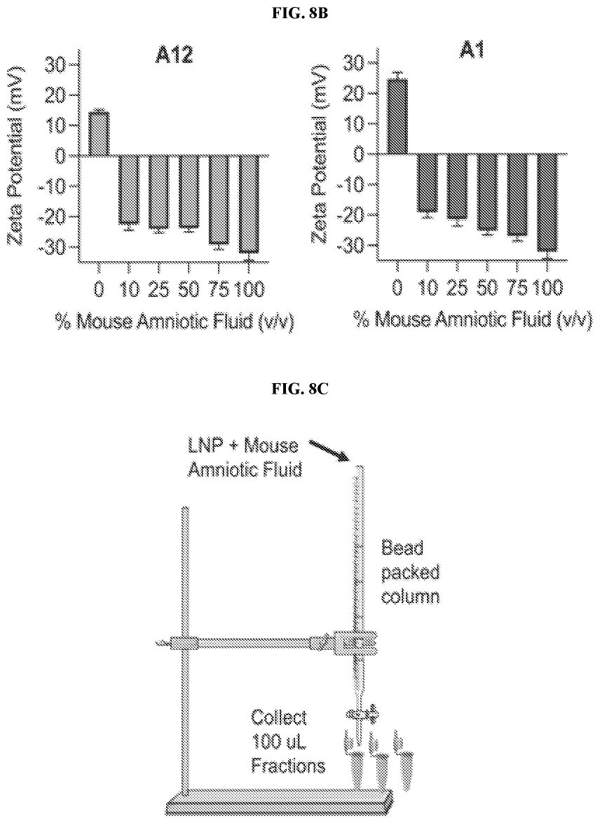

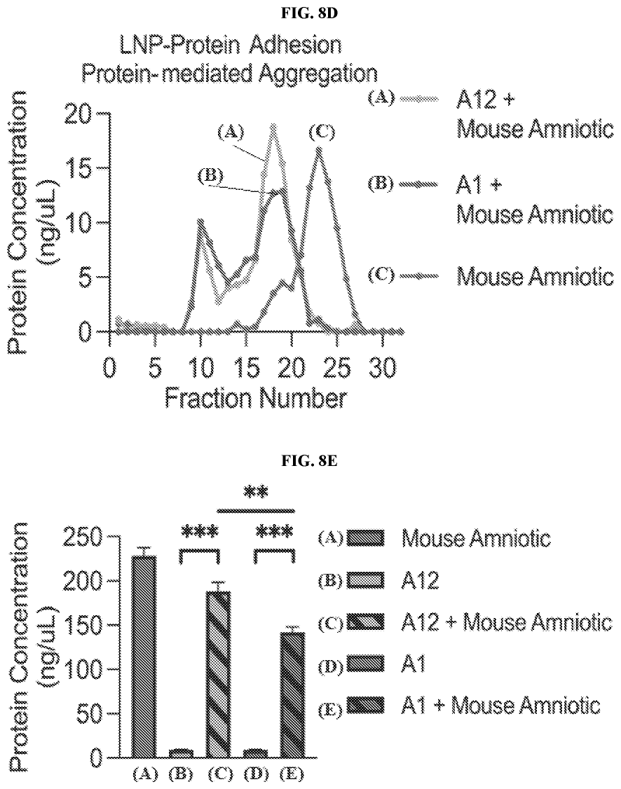

To visualize LNP morphological changes following incubation in mouse amniotic fluid, transmission electron microscopy (TEM) was used to visualize the morphology of the most stable (A12) and least stable (A1) LNPs from the above ex utero stability screen in mouse amniotic fluid. First, TEM images of LNPs A12 and A1 in PBS showed primarily spherical and monodisperse particles (FIG. 8A). Particle analysis of TEM images indicated A12 had a mean size of 97±17 nm and A1 had a mean size of 71±12 nm. Upon incubation in mouse amniotic fluid, TEM images of the most stable LNP (A12) showed little shape or size changes. However, the least stable LNP (A1) showed substantial aggregation and clustering in mouse amniotic fluid. These qualitative morphological changes are confirmed with TEM image particle analysis where the LNP size was 118±43 nm for A12 and 176±110 nm for A1 following incubation in mouse amniotic fluid.

To further characterize LNP-protein effects following incubation in mouse amniotic fluid, the zeta potential of LNPs A12 and A1 was measured following incubation in increasing fluids percentages of mouse amniotic fluid (FIG. 8B). Previous findings report that zeta potential measurements became more negative as NPs were incubated in increasing concentrations of protein-rich fluid (Grafe et al., 2016, Int. J. Biochem. Cell Biol. 75:196-202). Here, both LNPs A12 and A1 alone had positive zeta potential measurements that immediately became negative upon addition of mouse amniotic fluid. The zeta potential measurements became increasingly more negative as fluid percentage increased, as is consistent with previous findings, likely due to increased protein adhesion to the particle.

### Example 7: Chromatography and Protein Quantification of LNPs in Mouse Amniotic Fluid

As mouse serum and the amniotic fluids evaluated in this study are protein-rich biological environments, experiments were performed to identify the presence of bound proteins on LNPs A12 and A1 following incubation in mouse amniotic fluid. To do so, a previously reported methodology of Sepharose column separation to isolate LNPs from unbound protein-rich fluid (Amici et al., 2017, RSC Adv. 7:1137-1145) was expanded on. Using this protocol, free mouse amniotic fluid and LNPs A12 and A1 pre-incubated in mouse amniotic fluid were each individually passed through a Sepharose column (FIG. 8C). 32 chromatographic fractions were collected for each of the three samples, and the protein concentration of each fraction was measured using Tecan's NanoQuant Plate. Plots of protein concentration as a function of chromatographic fraction indicate the presence of two peaks for LNP samples. Without being bound by theory, it was hypothesized that the first peaks represent LNP aggregates which would be larger in size and elute from the separation column first before smaller LNPs with bound proteins on their surfaces, representing the second peaks detected in the chromatographic fractions.

Fractions with non-zero protein readings and no overlap with the elution of free mouse amniotic fluid were pooled and used to measure protein content via BCA assay. The BCA assay indicated significant (***p<0.0002) protein content on LNPs A12 and A1 that were pre-incubated in mouse amniotic fluid compared to the same LNPs in PBS alone. Interestingly, the most stable LNP (A12) from the ex utero mouse amniotic stability screen had significantly (***p<0.0021) higher protein content bound to the surface than the least stable (A1) LNP from the stability screen. This assay confirms that proteins derived from mouse amniotic fluid are bound to LNPs A12 and A1 following incubation, and these LNP-protein interactions likely contribute to the previous stability findings.

### Example 8: In Vitro LNP-mediated mRNA Delivery

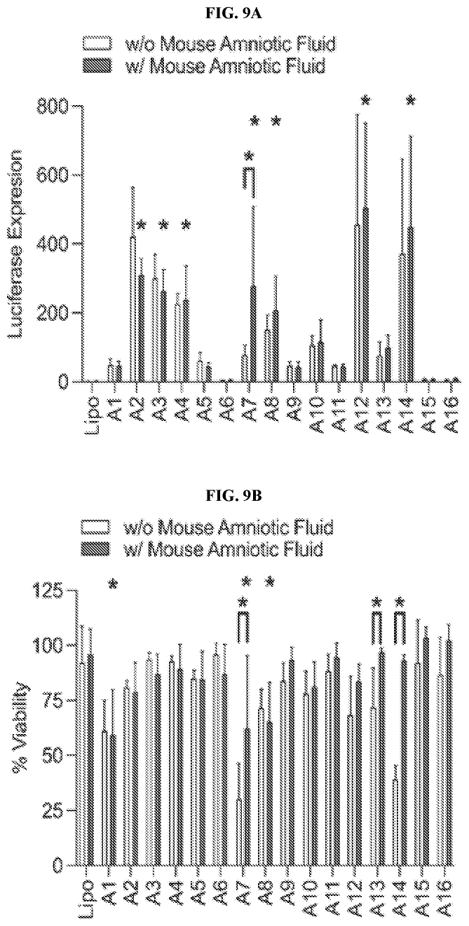

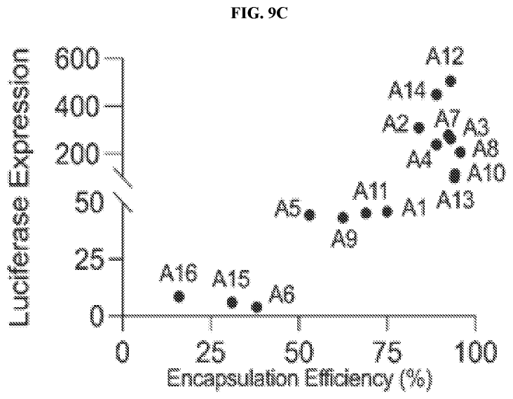

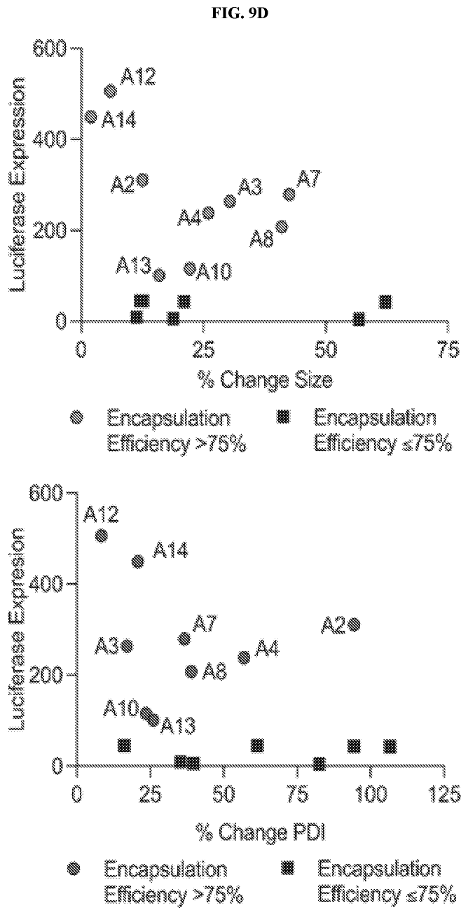

To establish trends between ex vivo LNP stability and mRNA delivery, LNP-mediated luciferase mRNA delivery and toxicity of the library in vitro in HeLa cells was evaluated (FIGS. 9A-9D). HeLa cells were selected for this in vitro library screen as epithelial cells are found in several major organ targets for intra-amniotic injection of LNPs, including the skin, pulmonary and digestive tract organs. Treatment conditions included LNPs alone and LNPs pre-incubated in mouse amniotic fluid. HeLa cells were dosed with LNPs or lipofectamine at a concentration of 50 ng per 10,000 cells. Lipofectamine is a commonly used transfection agent and is often considered the gold standard for in vitro delivery. To evaluate LNP delivery, 24 hours after treatment, luciferase expression was quantified using bioluminescence measurements (FIG. 9A). Seven LNPs—A2, A3, A4, A7, A8, A12, and A14 with mouse amniotic fluid had significantly (*p<0.05) higher luciferase expression than lipofectamine. For 15 of 16 LNPs in the library, there were no significant differences in luciferase expression between the LNP alone and the LNP with mouse amniotic fluid treatment conditions, except for A7 which demonstrated significantly (p<0.05) better delivery in the presence of mouse amniotic fluid. Percent cell viability was also evaluated 24 hours following treatment with LNPs or lipofectamine. LNPs A1, A7, and A8 with mouse amniotic fluid had significantly lower cell viability compared to lipofectamine (FIG. 9B). Notably, three LNPs—A7, A13, and A14—had significantly better cell viability in the presence of mouse amniotic fluid than in PBS alone.

As expected, luciferase expression demonstrated a strong correlation with encapsulation efficiency, as LNPs with less than or equal to 75% encapsulation had little to no delivery (FIG. 9C). Focusing on LNPs with encapsulation efficiencies greater than 75%, correlations were assessed between luciferase expression and percent change in size or percent change in PDI ex utero stability measurements in mouse amniotic fluid (FIG. 9D). A general inverse correlation was noted between luciferase expression and both percent change in size and percent change in PDI. A12, the most stable LNP in mouse amniotic fluid, had the highest luciferase expression of all LNPs evaluated in the library. These results confirm the ability of the stability measurements to predict top in vitro performers such as LNPs A12 or A14.

### Example 9: LNP Structure Function Relationships with Ex Utero Stability and In Vitro Delivery

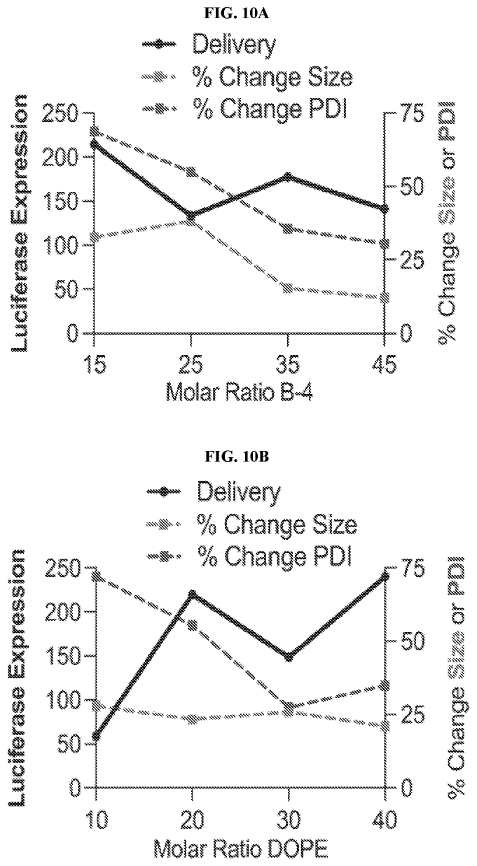

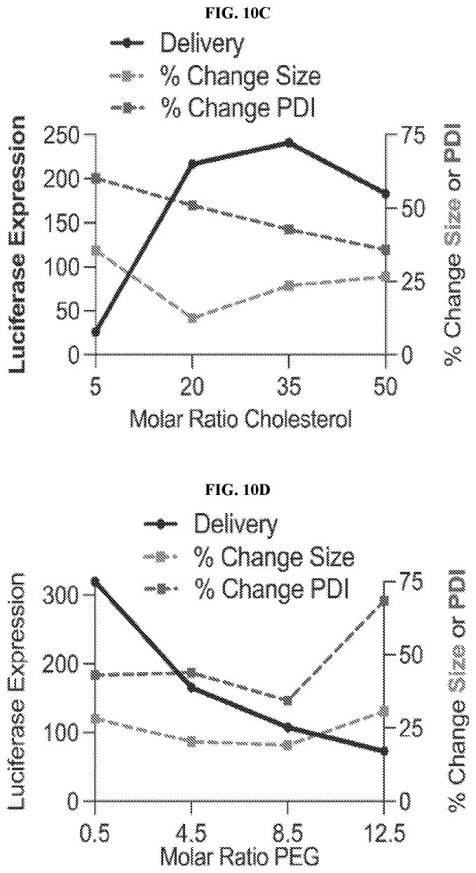

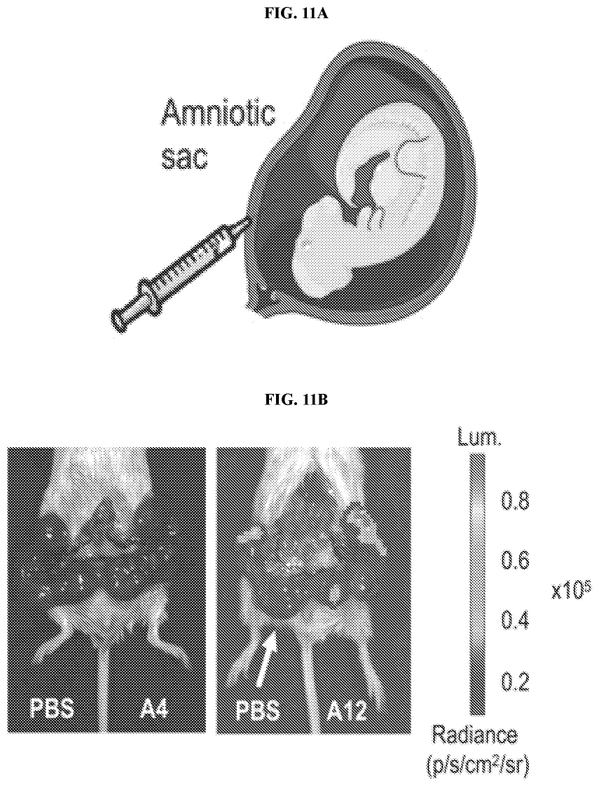

As the LNP library was designed with four molar ratio levels of each of four excipients, LNP structure function relationships were investigated with ex utero stability measurements and in vitro delivery (FIGS. 10A-10D). For ionizable lipid B-4, it was found that percent change in size and percent change in PDI decreased as molar ratio increased, yet there was no noticeable trend for delivery (FIG. 10A). For both DOPE and cholesterol, percent change in PDI decreased and luciferase expression increased as the excipient molar ratio increased (FIGS. 10B-10C). If the stability measurements are accurate predictors of mRNA delivery, it would be expected that trends such as these where percent change in size and percent change in PDI stability measurements should get smaller as delivery improves.

Finally, for PEG, percent change in PDI increased and luciferase expression decreased as the molar ratio of PEG increased (FIG. 10D). Again, an inverse trend was observed between the percent change in PDI stability measurements and luciferase delivery. As it appears that the percent change in PDI measurements track as expected with luciferase delivery, these results suggest that the PDI stability measurements might better predict in vitro delivery than percent change in size measurements. Additionally, these results help identify certain molar ratios (10 for DOPE, 5 for cholesterol, 12.5 for PEG) that confer LNP instability as seen by high percent change in PDI measurements and low luciferase delivery.

### Example 10: LNP Mediated Intra-Amniotic Luciferase mRNA Delivery

Two LNPs were selected to evaluate the ability of ex utero stability measurements in mouse amniotic fluid to predict intra-amniotic luciferase mRNA delivery to E16 fetal mice. Gestational age E16 fetuses were selected for intra-amniotic injection as E16 amniotic fluid was used in the previous ex utero stability measurements. Additionally, gestational age E16 represents the biological environment at the onset of fetal breathing for inhalation and ingestion of LNPs from the amniotic fluid. LNP A12 was selected as it was the most stable LNP in mouse amniotic fluid and had the highest in vitro luciferase mRNA delivery. LNP A4 was selected for its poorer ex utero stability in mouse amniotic fluid and lower in vitro delivery.

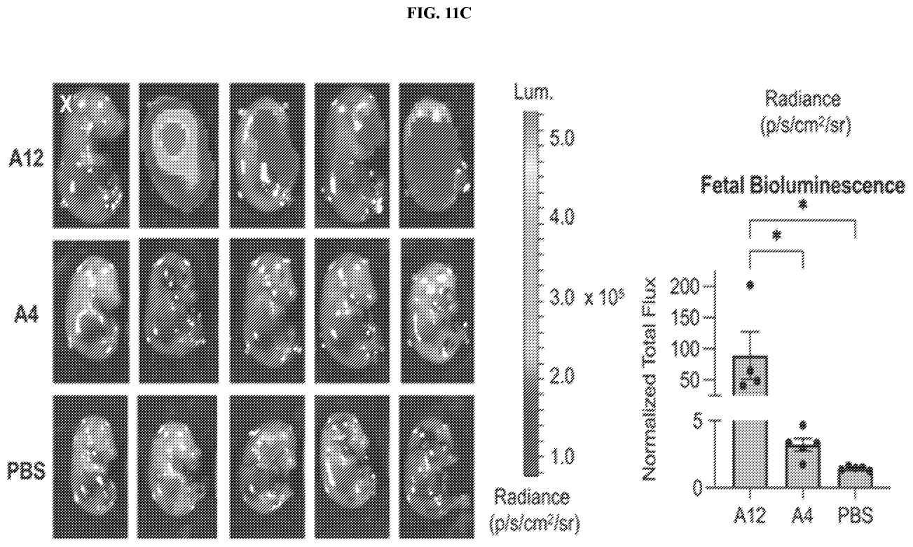

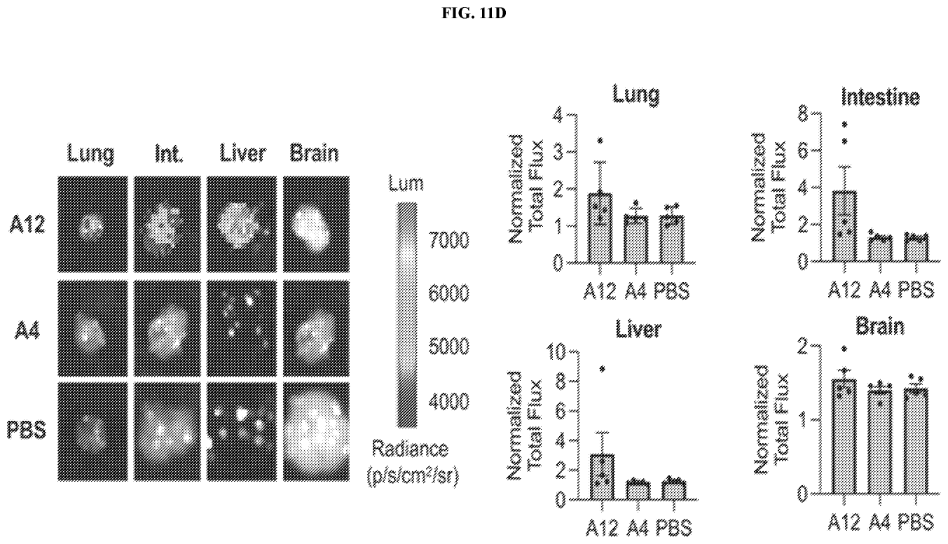

LNPs A12 and A4 were concentrated to 325 ng/μL and 30 μL of LNP or PBS was injected into five individual fetal amniotic sacs of E16 pregnant dams for each test condition (FIG. 11A). Four hours after injection, luciferin was administered to the dams and IVIS imaging was used to quantify luciferase expression. IVIS images of the dams and exposed uterine horns showed no luciferase delivery for sacs receiving PBS and A4 injections (FIG. 11B). In contrast, there was clear luminescence in sacs receiving A12 injections. Fetuses were removed and individually assessed by IVIS imaging. When quantified and averaged, fetal bioluminescence was significantly higher (*p<0.05) for fetuses receiving LNP A12 injections compared to both LNP A4 and PBS injections (FIG. 11C). There was no significant delivery of the A4 LNP compared to PBS control.

Finally, fetal organs including the lung, intestine, liver, and brain were isolated and assessed by IVIS for bioluminescence. Fetuses undergoing intra-amniotic injection with LNP A12 demonstrated luminescence in the lung and intestines as well as the liver, consistent with fetal swallowing and inhalation of the amniotic fluid containing LNP A12. In contrast, no luminescence was detected in any organs of those fetuses in which LNP A4 or PBS was injected into the amniotic sac. (FIG. 11D). Overall, this data demonstrates proof-of-concept that ex utero stability measurements in mouse amniotic fluid are a potential predictor for in utero intra-amniotic luciferase mRNA delivery.

### Enumerated Embodiments

The following exemplary embodiments are provided, the numbering of which is not to be construed as designating levels of importance:

Embodiment 1 provides a lipid nanoparticle (LNP) comprising:
a) an ionizable lipid compound or salt thereof having the structure of Formula (I):

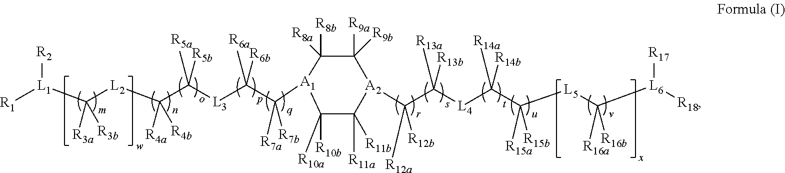

wherein:
   A₁ and A₂ is independently selected from the group consisting of C(H) and N;
   L₁ and L₆ are each independently selected from the group consisting of C(R₁₉) and N;
   each occurrence of L₂, L₃, L₄, and L₅, is independently selected from the group consisting of —C(H)₂—, —C(H)(R₁₉)—, —O—, —N(H)—, and —N(R₁₉)—;
   each occurrence of R₁, R₂, R₃ₐ, R_{3b}, R₄ₐ, R_{4b}, R₅ₐ, R_{5b}, R₆ₐ, R_{6b}, R₇ₐ, R_{7b}, R₈ₐ, R_{8b}, R₉ₐ, R_{9b}, R₁₀ₐ, R_{10b}, R₁₁ₐ, R_{11b}, R₁₂ₐ, R_{12b}, R₁₃ₐ, R_{13b}, R₁₄ₐ, R_{14b}, R₁₅ₐ, R_{15b}, R₁₆, R₁₇, R₁₈, and R₁₉ is independently selected from the group consisting of H, OH, ═O, CN, NO₂, halogen, optionally substituted C₁-C₂₄ alkyl, optionally substituted C₃-C₁₂ cycloalkyl, —Y(R₂₀)_{z′}(R₂₁)_{z″}-(optionally substituted C₃-C₁₂ cycloalkyl), optionally substituted C₂-C₁₂ heterocycloalkyl, —Y(R₂₀)_{z′}(R₂₁)_{z″}-(optionally substituted C₂-C₁₂ heterocycloalkyl), optionally substituted C₂-C₂₄ alkenyl, optionally substituted C₅-C₁₂ cycloalkenyl, —Y(R₂₀)_{z′}(R₂₁)_{z″}-(optionally substituted C₂-C₁₂ cycloalkenyl), optionally substituted C₂-C₂₄ alkynyl, optionally substituted C₅-C₁₂ cycloalkynyl, —Y(R₂₀)_{z′}(R₂₁)_{z″}-(optionally substituted C₅-C₁₂ cycloalkynyl), optionally substituted C₆-C₁₂ aryl, —Y(R₂₀)_{z′}(R₂₁)_{z″}-(optionally substituted C₆-C₁₂ aryl), optionally substituted C₂-C₁₂ heteroaryl, —Y(R₂₀)_{z′}(R₂₁)_{z″}-(optionally substituted C₂-C₁₂ heteroaryl), —C(═O)OH, —C(═O)O(optionally substituted C₁-C₂₄ alkyl), —C(═O)O(optionally substituted C₂-C₂₄ alkenyl), —C(═O)O(optionally substituted C₆-C₁₂ aryl), —C(═O)O(optionally substituted C₂-C₁₂ heteroaryl), amido, amino, —Y(R₂₀)_{z′}(R₂₁)_{z″}—C(═O)O(optionally substituted C₁-C₂₄ alkyl), —Y(R₂₀)_{z′}(R₂₁)_{z″}—C(═O)O(optionally substituted C₂-C₂₄ alkenyl), —Y(R₂₀)_{z′}(R₂₁)_{z″}—C(═O)O(optionally substituted C₆-C₁₂ aryl), and —Y(R₂₀)_{z′}(R₂₁)_{z″}—C(═O)O(optionally substituted C₂-C₁₂ heteroaryl);
   wherein Y is selected from the group consisting of C, N, O, S, and P;
   wherein each R₂₀ and R₂₁ is independently selected from the group consisting of H, OH, ═O, NO₂, CN, halogen, optionally substituted C₁-C₂₄ alkyl, optionally substituted C₃-C₁₂ cycloalkyl, optionally substituted C₂-C₁₂ heterocycloalkyl, optionally substituted C₂-C₂₄ alkenyl, optionally substituted C₅-C₁₂ cycloalkenyl, optionally substituted C₂-C₂₄ alkynyl, optionally substituted C₅-C₁₂ cycloalkynyl, optionally substituted C₆-C₁₂ aryl, optionally substituted C₂-C₁₂ heteroaryl, —C(═O)OH, —C(═O)O(optionally substituted C₁-C₂₄ alkyl), —C(═O)O(optionally substituted C₂-C₂₄ alkenyl), —C(═O)O(optionally substituted C₆-C₁₂ aryl), —C(═O)O(optionally substituted C₂-C₁₂ heteroaryl), amido, amino, —C(═O)(optionally substituted C₁-C₂₄ alkyl), —C(═O)(optionally substituted C₂-C₂₄ alkenyl), —C(═O)(optionally substituted C₆-C₁₂ aryl), and —C(═O)(optionally substituted C₂-C₁₂ heteroaryl);
   or R₂₀ and R₂₁ may combine with Y to form a —(C═O)—;
   wherein z′ and z″ are each independently an integer represented by 0, 1, or 2; and
   wherein m, n, o, p, q, r, s, t, u, v, w, and x are each independently an integer represented by 0, 1, 2; 3, 4, or 5;
   wherein the compound or salt thereof having the structure of Formula (I) is present in a concentration range of about 10 mol % to about 50 mol %;
b) dioleoyl-phosphatidylethanolamine (DOPE), wherein the DOPE is present in a concentration range of about 10 mol % to about 45 mol %;
c) a cholesterol lipid in a concentration range of about 5 mol % to about 50 mol %; and
d) polyethylene glycol (PEG) in a concentration range of about 0.5 mol % to about 12.5 mol %.

Embodiment 2 provides the LNP of Embodiment 1, wherein each occurrence of R₁, R₂, R₁₇, R₁₈, and R₁₉ is independently selected from the group consisting of H and —CH₂—CH(OH)—(C₁-C₂₂ alkyl).

Embodiment 3 provides the LNP of Embodiment 1 or 2, wherein each occurrence of R₁, R₂, R₁₇, R₁₈, and R₁₉ is independently selected from the group consisting of H and —CH₂—CH(OH)—(CH₂)₁₁CH₃.

Embodiment 4 provides the LNP of any one of Embodiments 1-3, wherein the ionizable lipid comprises:

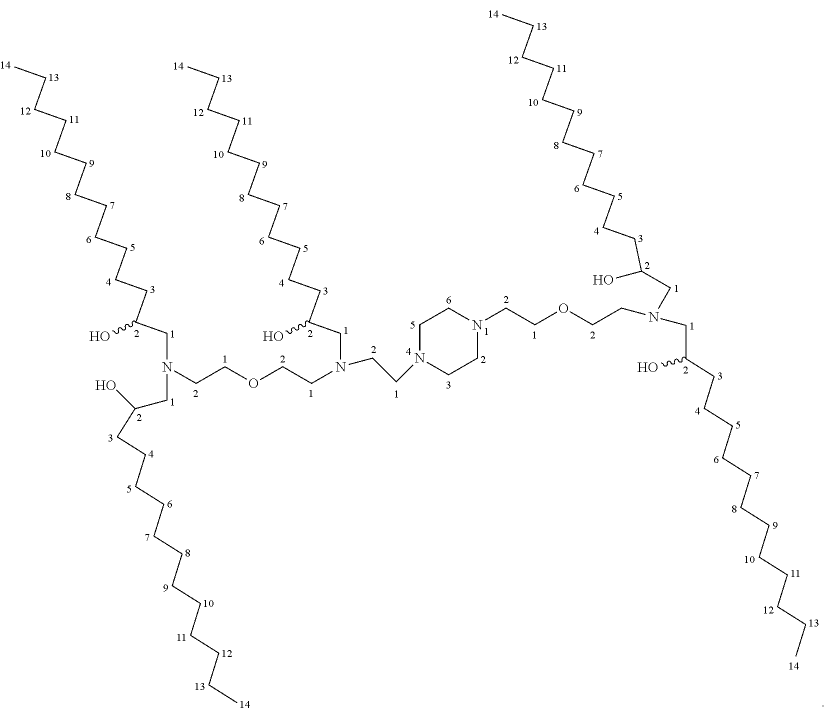 [SMILES: CCCCCCCCCCCCC(O)CN(CCOCCN(CC(O)CCCCCCCCCCCC)CC(O)CCCCCCCCCCCC)CCN1CCN(CCOCCN(CC(O)CCCCCCCCCCCC)CC(O)CCCCCCCCCCCC)CC1]

Embodiment 5 provides the LNP ofany one of Embodiments 1-4 wherein the molar ratio of (a):(b):(c):(d) in Embodiment 1 is selected from the group consisting of:
(a) about 15:10:5:0.5;
(b) about 15:20:20:4.5;
(c) about 15:40:50:12.5;
(d) about 35:30:5:4.5;
(e) about 35:40:20:0.5;
(f) about 45:20:35:0.5; and
(g) about 45:40:5:8.5.

Embodiment 6 provides the LNP of any one of Embodiments 1-5, wherein the LNP further comprises at least one selected from the group consisting of a nucleic acid molecule, therapeutic agent, and any combination thereof.

Embodiment 7 provides the LNP of Embodiment 6, wherein the nucleic acid molecule is a therapeutic agent.

Embodiment 8 provides the LNP of any one of Embodiments 6-7, wherein the nucleic acid molecule is a DNA molecule or an RNA molecule.

Embodiment 9 provides the LNP of any one of Embodiments 6-8, wherein the nucleic acid molecule is selected from the group consisting of cDNA, mRNA, miRNA, siRNA, modified RNA, antagomir, antisense molecule, peptide, therapeutic peptide, targeted nucleic acid, and any combination thereof.

Embodiment 10 provides the LNP of any one of Embodiments 6-9, wherein the nucleic acid molecule encodes one or more components for gene editing.

Embodiment 11 provides the LNP of any one of Embodiments 6-10, wherein the mRNA encodes one or more antigens.

Embodiment 12 provides a composition comprising at least one LNP of any one of Embodiments 1-11 and a pharmaceutically acceptable carrier.

Embodiment 13 provides the composition of Embodiment 12, wherein the composition further comprises an adjuvant.

Embodiment 14 provides the composition of Embodiment 13, wherein the adjuvant is at least one selected from the group consisting of squalene, a TLR7 agonist, and a TLR8 agonist.

Embodiment 15 provides the composition of any one of Embodiments 12-14, wherein the composition is a vaccine.

Embodiment 16 provides a method of delivering a nucleic acid molecule, therapeutic agent, or a combination thereof to a fetal subject in utero, the method comprising administering a therapeutically effectively amount of at least one LNP of any one of Embodiments 1-11 and/or at least one composition of any one of Embodiments 12-15 to a maternal subject comprising the fetal subject.

Embodiment 17 provides the method of Embodiment 16, wherein the nucleic acid molecule is a therapeutic agent.

Embodiment 18 provides the method of Embodiment 16 or 17, wherein the nucleic acid molecule is a DNA molecule or an RNA molecule.

Embodiment 19 provides the method of any one of Embodiments 16-18, wherein the nucleic acid molecule is selected from the group consisting of cDNA, mRNA, miRNA, siRNA, antagomir, antisense molecule, peptide, therapeutic peptide, targeted nucleic acid, and any combination thereof.

Embodiment 20 provides the method of any one of Embodiments 16-19, wherein the nucleic acid molecule encodes one or more components for gene editing.

Embodiment 21 provides the method of any one of Embodiments 16-20, wherein the mRNA encodes one or more antigens.

Embodiment 22 provides the method of any one of Embodiments 16-21, wherein the LNP of any one of Embodiments 1-11 or the composition of any one of Embodiments 12-15 further comprises an adjuvant.

Embodiment 23 provides the method of any one of Embodiments 16-22, wherein the nucleic acid molecule, therapeutic agent, or combination thereof is encapsulated within the LNP.

Embodiment 24 provides the method of any one of Embodiments 16-23, wherein the LNP or the composition thereof is administered by in utero delivery.

Embodiment 25 provides the method of any one of Embodiments 16-24, wherein the method treats or prevents at least one selected from the group consisting of a viral infection, a bacterial infection, a fungal infection, a parasitic infection, influenza infection, cancer, arthritis, heart disease, cardiovascular disease, neurological disorder or disease, genetic disease, autoimmune disease, and fetal disease, genetic disease affecting fetal development, and any combination thereof.

Embodiment 26 provides a method of preventing, ameliorating, and/or treating a disease or disorder in a target fetal subject, the method comprising in utero administering a therapeutically effectively amount of at least one LNP of any one of Embodiments 1-11 or at least one composition of any one of Embodiments 12-15 to a maternal subject comprising the fetal subject.

Embodiment 27 provides the method of Embodiment 26, wherein the LNP or the composition thereof delivers the nucleic acid molecule, therapeutic agent, or combination thereof to a fetal cell.

Embodiment 28 provides the method of Embodiment 26 or 27, wherein the disease or disorder is selected from the group consisting of β-thalassemia, cystic fibrosis, glycogen storage disorders, cleft lip and cleft palate, cerebral palsy, Fragile X syndrome, Down syndrome, spina bifida, congenital heart defects, genetic lung diseases, genetic skin diseases, amniotic membrane rupture, and amniotic membrane diseases.

Embodiment 29 provides a method of delivering a nucleic acid molecule to a fetal cell, the method comprising in utero administration of a therapeutically effectively amount of at least one LNP of any one of Embodiments 1-11 or at least one composition of any one of Embodiments 12-15 comprising to a maternal subject comprising the fetal cell.

Embodiment 30 provides the method of Embodiment 29, wherein the method is a gene delivery method.

Embodiment 31 provides a method of identifying a LNP as having increased stability in a test fluid, the method comprising:
a) contacting at least one LNP with a first concentration of a test fluid;
b) determining the size and polydispersity index (PDI) of the LNP using dynamic light scattering (DLS);
c) contacting the at least one LNP to be tested with at least one additional concentration of the fluid;
d) determining the size and polydispersity index (PDI) of the LNP using dynamic light scattering (DLS) in the presence of the at least one additional concentration of the fluid;
e) comparing the size and polydispersity index (PDI) of the LNP at the at least two fluid concentrations; and
f) identifying the test LNP as having stability in the test fluid based on the changes in size and polydispersity index (PDI) of the LNP.

Embodiment 32 provides the method of Embodiment 31, wherein the test fluid mimics a biological fluid.

Embodiment 33 provides the method of Embodiment 31 or 32, wherein the biological fluid is amniotic fluid or an amniotic fluid mimic.

Embodiment 34 provides the method of any one of Embodiments 31-33, wherein the method is a screening method for detecting LNPs having increased stability.

Embodiment 35 provides the method of any one of Embodiments 31-34, wherein the test LNP is identified as having stability in the test fluid based a lower level of change in at least one selected from the group consisting of size and PDI of the LNP as compared to a control LNP.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

1. A lipid nanoparticle (LNP) comprising:
a) an ionizable lipid compound or salt thereof having the structure of Formula (I): 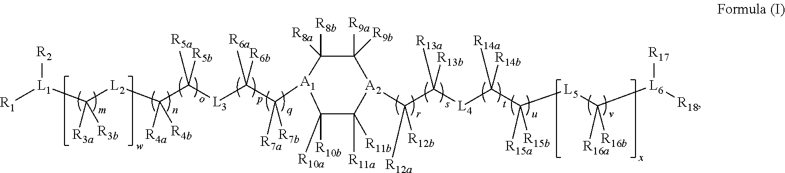
wherein:
A₁ and A₂ are each independently selected from the group consisting of C(H) and N;
L₁ and L₆ are each independently selected from the group consisting of C(R₁₉) and N;
each occurrence of L₂, L₃, L₄, and L₅, is independently selected from the group consisting of —C(H)₂—, —C(H)(R₁₉)—, —O—, —N(H)—, and —N(R₁₉)—;
each occurrence of R₁, R₂, R₃ₐ, R_{3b}, R₄ₐ, R_{4b}, R₅ₐ, R_{5b}, R₆ₐ, R_{6b}, R₇ₐ, R_{7b}, R₈ₐ, R_{8b}, R₉ₐ, R_{9b}, R₁₀ₐ, R_{10b}, R₁₁ₐ, R_{11b}, R₁₂ₐ, R_{12b}, R₁₃ₐ, R_{13b}, R₁₄ₐ, R_{14b}, R₁₅ₐ, R_{15b}, R₁₆ₐ, R_{16b}, R₁₇, R₁₈, and R₁₉ is independently selected from the group consisting of H, OH, ═O, CN, NO₂, halogen, optionally substituted C₁-C₂₄ alkyl, optionally substituted C₃-C₁₂ cycloalkyl, —Y(R₂₀)_{z′}(R₂₁)_{z″}-(optionally substituted C₃-C₁₂ cycloalkyl), optionally substituted C₂-C₁₂ heterocycloalkyl, —Y(R₂₀)_{z′}(R₂₁)_{z″}-(optionally substituted C₂-C₁₂ heterocycloalkyl), optionally substituted C₂-C₂₄ alkenyl, optionally substituted C₅-C₁₂ cycloalkenyl, —Y(R₂₀)_{z′}(R₂₁)_{z″}-(optionally substituted C₂-C₁₂ cycloalkenyl), optionally substituted C₂-C₂₄ alkynyl, optionally substituted C₈-C₁₂ cycloalkynyl, —Y(R₂₀)_{z′}(R₂₁)_{z″}-(optionally substituted C₈-C₁₂ cycloalkynyl), optionally substituted C₆-C₁₂ aryl, —Y(R₂₀)_{z′}(R₂₁)_{z″}-(optionally substituted C₆-C₁₂ aryl), optionally substituted C₂-C₁₂ heteroaryl, —Y(R₂₀)_{z′}(R₂₁)_{z″}-(optionally substituted C₂-C₁₂ heteroaryl), —C(═O)OH, —C(═O)O(optionally substituted C₁-C₂₄ alkyl), —C(═O)O(optionally substituted C₂-C₂₄ alkenyl), —C(═O)O(optionally substituted C₆-C₁₂ aryl), —C(═O)O(optionally substituted C₂-C₁₂ heteroaryl), amido, amino, —Y(R₂₀)_{z′}(R₂₁)_{z″}—C(═O)O(optionally substituted C₁-C₂₄ alkyl), —Y(R₂₀)_{z′}(R₂₁)_{z″}—C(═O)O(optionally substituted C₂-C₂₄ alkenyl), —Y(R₂₀)_{z′}(R₂₁)_{z″}—C(═O)O(optionally substituted C₆-C₁₂ aryl), and —Y(R₂₀)_{z′}(R₂₁)_{z″}—C(═O)O(optionally substituted C₂-C₁₂ heteroaryl);
wherein Y is selected from the group consisting of C, N, O, S, and P;
wherein each R₂₀ and R₂₁ is independently selected from the group consisting of H, OH, ═O, NO₂, CN, halogen, optionally substituted C₁-C₂₄ alkyl, optionally substituted C₃-C₁₂ cycloalkyl, optionally substituted C₂-C₁₂ heterocycloalkyl, optionally substituted C₂-C₂₄ alkenyl, optionally substituted C₅-C₁₂ cycloalkenyl, optionally substituted C₂-C₂₄ alkynyl, optionally substituted C₈-C₁₂ cycloalkynyl, optionally substituted C₆-C₁₂ aryl, optionally substituted C₂-C₁₂ heteroaryl, —C(═O)OH, —C(═O)O(optionally substituted C₁-C₂₄ alkyl), —C(═O)O(optionally substituted C₂-C₂₄ alkenyl), —C(═O)O(optionally substituted C₆-C₁₂ aryl), —C(═O)O(optionally substituted C₂-C₁₂ heteroaryl), amido, amino, —C(═O)(optionally substituted C₁-C₂₄ alkyl), —C(═O)(optionally substituted C₂-C₂₄ alkenyl), —C(═O)(optionally substituted C₆-C₁₂ aryl), and —C(═O)(optionally substituted C₂-C₁₂ heteroaryl);
or R₂₀ and R₂₁ may combine with Y to form —(C═O)—;
wherein z′ and z″ are each independently 0, 1, or 2; and
wherein m, n, o, p, q, r, s, t, u, v, w, and x are each independently 0, 1, 2; 3, 4, or 5;
wherein the compound or salt thereof having the structure of Formula (I) is present in a concentration range of about 10 mol % to about 50 mol % in the LNP;
b) dioleoyl-phosphatidylethanolamine (DOPE), wherein the DOPE is present in a concentration range of about 10 mol % to about 45 mol % in the LNP;
c) a cholesterol lipid in a concentration range of about 5 mol % to about 50 mol % in the LNP; and
d) a polyethylene glycol (PEG)-conjugated lipid in a concentration range of about 0.5 mol % to about 12.5 mol % in the LNP.

2. The LNP of claim 1, wherein each occurrence of R₁, R₂, R₁₇, R₁₈, and R₁₉ is independently selected from the group consisting of H and —CH₂—CH(OH)—(C₁-C₂₂ alkyl), optionally wherein each occurrence of R₁, R₂, R₁₇, R₁₈, and R₁₉ is independently selected from the group consisting of H and —CH₂—CH(OH)—(CH₂)₁₁CH₃.

3. (canceled)

4. The LNP of claim 1, wherein the ionizable lipid comprises:

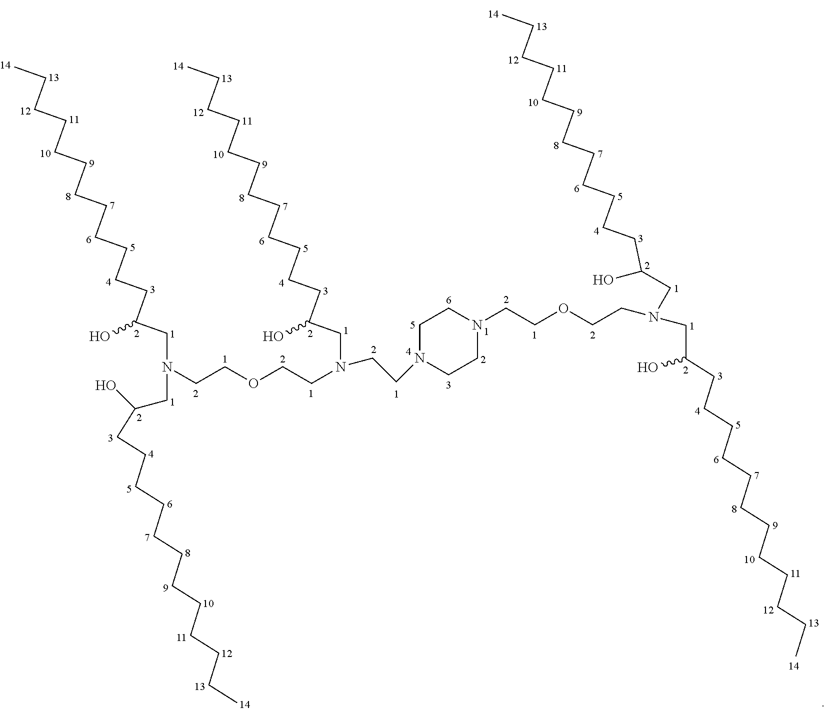 [SMILES: CCCCCCCCCCCCC(O)CN(CCOCCN(CC(O)CCCCCCCCCCCC)CC(O)CCCCCCCCCCCC)CCN1CCN(CCOCCN(CC(O)CCCCCCCCCCCC)CC(O)CCCCCCCCCCCC)CC1]

5. The LNP of claim 1, wherein the molar ratio of (a):(b):(c):(d) in claim 1 is selected from the group consisting of:
(a) about 15:10:5:0.5;
(b) about 15:20:20:4.5;
(c) about 15:40:50:12.5;
(d) about 35:30:5:4.5;
(e) about 35:40:20:0.5;
(f) about 45:20:35:0.5; and
(g) about 45:40:5:8.5.

6. The LNP of claim 1, wherein the LNP further comprises at least one selected from the group consisting of a nucleic acid molecule, therapeutic agent, and any combination thereof, optionally wherein the nucleic acid molecule is a DNA or RNA molecule, and optionally wherein the nucleic acid molecule is selected from the group consisting of cDNA, mRNA, miRNA, siRNA, modified RNA, antagomir, antisense molecule, peptide, therapeutic peptide, targeted nucleic acid, and any combination thereof.

7-9. (canceled)

10. The LNP of claim 6, wherein the nucleic acid molecule encodes one or more components for gene editing or wherein the nucleic acid encodes one or more antigens.

11. (canceled)

12. A composition comprising at least one LNP of claim 1 and a pharmaceutically acceptable carrier, optionally wherein the composition is a vaccine.

13. The composition of claim 12, wherein the composition further comprises an adjuvant, optionally wherein the adjuvant is at least one selected from the group consisting of squalene, a TLR7 agonist, and a TLR8 agonist.

14-15. (canceled)

16. A method of delivering a nucleic acid molecule, therapeutic agent, or a combination thereof to a fetal subject in utero, the method comprising administering a therapeutically effectively amount of at least one LNP of claim 1 or a pharmaceutical composition thereof further comprising at least one pharmaceutically acceptable carrier to a maternal subject comprising the fetal subject.

17. The method of claim 16, wherein the nucleic acid molecule is a therapeutic agent, optionally wherein the nucleic acid molecule is a DNA or RNA molecule, and optionally wherein the nucleic acid molecule is selected from the group consisting of cDNA, mRNA, miRNA, siRNA, modified RNA, antagomir, antisense molecule, peptide, therapeutic peptide, targeted nucleic acid, and any combination thereof.

18-19. (canceled)

20. The method of claim 16, wherein at least one of the following applies:
(a) the nucleic acid molecule encodes one or more components for gene editing;
(b) the nucleic acid molecule encodes one or more antigens;
(c) the LNP or pharmaceutical composition thereof further comprises an adjuvant;
(d) the nucleic acid molecule, therapeutic agent, or combination thereof is encapsulated within the LNP; and
(e) the LNP of pharmaceutical composition thereof is administered by in utero delivery.

21-24. (canceled)

25. The method of claim 16, wherein the method treats, ameliorates, or prevents at least one selected from the group consisting of a viral infection, a bacterial infection, a fungal infection, a parasitic infection, influenza infection, cancer, arthritis, heart disease, cardiovascular disease, neurological disorder or disease, genetic disease, autoimmune disease, and fetal disease, genetic disease affecting fetal development, and any combination thereof.

26. A method of preventing, ameliorating, or treating a disease or disorder in a target fetal subject, the method comprising in utero administering a therapeutically effectively amount of at least one LNP of claim 1 or a pharmaceutical composition thereof further comprising at least one pharmaceutically acceptable carrier to a maternal subject comprising the fetal subject.

27. The method of claim 26, wherein the LNP or the composition thereof delivers the nucleic acid molecule, therapeutic agent, or combination thereof to a fetal cell.

28. The method of claim 26, wherein the disease or disorder is selected from the group consisting of β-thalassemia, cystic fibrosis, glycogen storage disorders, cleft lip and cleft palate, cerebral palsy, Fragile X syndrome, Down syndrome, spina bifida, congenital heart defects, genetic lung diseases, genetic skin diseases, amniotic membrane rupture, and amniotic membrane diseases.

29. A method of delivering a nucleic acid molecule to a fetal cell, the method comprising in utero administration of a therapeutically effectively amount of at least one LNP of claim 1 or a pharmaceutical composition thereof further comprising at least one pharmaceutically acceptable carrier comprising to a maternal subject comprising the fetal cell.

30. The method of claim 29, wherein the method is a gene delivery method.

31. A method of identifying a lipid nanoparticle (LNP) as having increased stability in a test fluid, the method comprising:
a) contacting at least one LNP to be tested with a first concentration of a test fluid;
b) determining the size and polydispersity index (PDI) of the LNP using dynamic light scattering (DLS);
c) contacting the at least one LNP to be tested with at least one additional concentration of the fluid;
d) determining size and polydispersity index (PDI) of the LNP using dynamic light scattering (DLS) in the presence of the at least one additional concentration of the fluid;
e) comparing size and polydispersity index (PDI) of the LNP at the at least two fluid concentrations; and
f) identifying a test LNP as having stability in the test fluid based on changes in size and polydispersity index (PDI) of the LNP with the first concentration of test fluid and with the at least one additional concentration of the fluid.

32. The method of claim 31, wherein the test fluid mimics a biological fluid, optionally wherein the biological fluid is amniotic fluid or an amniotic fluid mimic.

33. (canceled)

34. The method of claim 31, wherein at least one of the following applies:
(a) the method is a screening method for detecting LNPs having increased stability; and
(b) the test LNP is identified as having stability in the test fluid based on a lower level of change in at least one selected from the group consisting of size and PDI of the LNP as compared to a control LNP.

35. (canceled)
